(19) United States
(12) Patent Application Publication
(10) Pub. No.: US 2023/0063426 A1
(43) Pub. Date: Mar. 2, 2023

(54) HETEROLOGOUS PROTEASE EXPRESSION FOR IMPROVING ALCOHOLIC FERMENTATION

(71) Applicant: Lallemand Hungary Liquidity Management LLC, Budapest (HU)

(72) Inventors: Trisha Barrett, Bradford, VT (US); Charles F. Rice, Plainfield, NH (US); Aaron Argyros, Lebanon, NH (US)

(21) Appl. No.: 17/929,986

(22) Filed: Sep. 6, 2022

Related U.S. Application Data
(63) Continuation of application No. 16/482,633, filed on Jul. 31, 2019, now abandoned.
(60) Provisional application No. 62/453,940, filed on Feb. 2, 2017.

(51) Int. Cl. C12N 9/60; C12N 1/18; C12P 7/06

(52) U.S. Cl. CPC C12N 9/60; C12N 1/18; C12P 7/06

(57) ABSTRACT

The present disclosure relates to proteases for improving alcoholic fermentation. The proteases are expressed from a recombinant host cell. The present disclosure also provides a population of recombinant host cells expressing an heterologous protease that can be used in combination with recombinant host cells expressing an heterologous glucoamylase and/or an heterologous glycerol reduction system.

### REFERENCE TO AN ELECTRONIC SEQUENCE LISTING

The contents of the electronic sequence listing (580127_406C1_SEQUENCE_LISTING.xml; Size: 224,621 bytes; and Date of Creation: Sep. 6, 2022) is herein incorporated by reference in its entirety.

### TECHNOLOGICAL FIELD

The present disclosure relates to the heterologous polypeptides, especially heterologous proteases, for improving alcoholic fermentation.

### BACKGROUND

Saccharomyces cerevisiae is used in the commercial production of distilled spirits and fuel ethanol. This organism is proficient in fermenting glucose to ethanol, often to concentrations greater than 20% w/v. However, S. cerevisiae's ability to generate a nitrogen source is limited which either slows down fermentation (for distilled spirits production) or requires the exogenous addition of nitrogen sources such as urea (for bioethanol production).

Corn is a feedstock for both distilled spirits and fuel ethanol. In the mashing process, corn is both thermally and enzymatically liquefied using α- or beta amylase prior to fermentation in order to break down long chain starch polymers into smaller dextrins. This can come either through addition of an external enzyme preparation, or as in with distilled spirits, through the addition of malted barley The mash is then cooled and inoculated with S. cerevisiae along with the exogenous addition of purified glucoamylase, an exo-acting enzyme, which will further break down the dextrin into utilizable glucose molecules.

It has been shown that the addition of commercial proteases such as FERMGEN® increases the rate of fermentation by supplying free amino acids via hydrolysis of protein found in the corn along with a decrease in the supply of additional nitrogen, resulting in a cost savings up to 4 cents per gallon (Johnston and McAloon, 2014). Adequate nitrogen content and other yeast nutrients contribute to the overall efficiency of the corn fermentation. Along with being a source of free amino nitrogen, protein is also a major component within the binding matrix of corn. Currently, commercial proteases are added to these industrial fermentations, which can be costly to corn ethanol plants. Addition of protease to the fermentation can also increase the ethanol yield (Johnston and McAloon, 2014), so even small increases such as 1% can translate into an extra billion gallons of ethanol per year.

There is thus a need to provide alternative fermenting materials and processes to improve alcoholic fermentation by increase the available nitrogen to the fermenting organisms.

### BRIEF SUMMARY

The present disclosure relates to the use of heterologous proteases expressed from a recombinant yeast host cell for improving alcoholic fermentation. In some embodiments, the heterologous proteases increases the fermentation rate, increases ethanol yields and/or decreases the production of glycerol by the fermenting recombinant host cells.

According to a first aspect, the present disclosure provides a first recombinant yeast host cell comprising a first genetic modification allowing the expression of an heterologous protease. The heterologous protease can be a polypeptide having the amino acid sequence of SEQ ID NO: 2, 6, 8, 10, 12, 14, 30, 36, 38, 40, 42, 52 or 92. The heterologous protease can be a variant having at least 70% identity to the amino acid sequence of SEQ ID NO: 2, 6, 8, 10, 12, 14, 30, 36, 38, 40, 42, 52 or 92 and exhibiting proteolytic activity. The heterologous protease can be a fragment having at least 70% identity to the amino acid sequence of SEQ ID NO: 2, 6, 8, 10, 12, 14, 30, 36, 38, 40, 42, 52 or 92 or the variant described herein and exhibiting proteolytic activity. In an embodiment, the heterologous protease is the polypeptide having the amino acid sequence of SEQ ID NO: 2, 14, 40 or 52. In still another embodiment, the heterologous protease is the variant of the polypeptide having the amino acid sequence of SEQ ID NO: 2, 14, 40 or 52. In still another embodiment, the heterologous protease is the fragment of the polypeptide having the amino acid sequence of SEQ ID NO: 2, 14, 40 or 52. In a further embodiment, the heterologous protease has the amino acid sequence of SEQ ID NO: 2, is the variant of the polypeptide of SEQ ID NO: 2 or is the fragment of the polypeptide of SEQ ID NO: 2. In still another embodiment, the heterologous protease has the amino acid sequence of SEQ ID NO: 14, is the variant of the polypeptide of SEQ ID NO: 14 or is the fragment of the polypeptide of SEQ ID NO: 14. In yet another embodiment, the heterologous protease has the amino acid sequence of SEQ ID NO: 40, is the variant of the polypeptide of SEQ ID NO: 40 or is the fragment of the polypeptide of SEQ ID NO: 40. In a further embodiment, the heterologous protease has the amino acid sequence of SEQ ID NO: 52, is the variant of the polypeptide of SEQ ID NO: 52 or is the fragment of the polypeptide of SEQ ID NO: 52. In yet another embodiment, the first recombinant yeast host cell has a second genetic modification allowing the expression of an heterologous glucoamylase, such as, for example, the heterologous glucoamylase has the amino acid sequence of SEQ ID NO: 91, is a variant of the amino acid sequence of SEQ ID NO: 91 or is a fragment of the amino acid sequence of SEQ ID NO: 91 or of the variant described herein. In still a further embodiment, the first recombinant yeast host cell has a third genetic modification for reducing the production of one or more native enzymes that function to produce glycerol or regulate glycerol synthesis. In some embodiments, the third genetic modification is for reducing the production of one or more native enzymes that function to produce glycerol, such as, for example, wherein the third genetic modification is for reducing or inhibiting in the expression of the gene encoding the GPD2 polypeptide. In yet another embodiment, the first recombinant yeast host cell has a fourth genetic modification for reducing the production of one or more native enzymes that function to catabolize formate, such as, for example, wherein the fourth genetic modification is for reducing or inhibiting the expression of the genes encoding the FDH1 polypeptide and the FDH2 polypeptide. In an embodiment, the first recombinant yeast host cell is from the genus Saccharomyces, such as, for example from the species Saccharomyces cerevisiae.

According to a second aspect, the present disclosure provides a cellular population comprising a first recombinant yeast host cell comprising the first genetic modification defined herein and a second recombinant yeast host cell comprising the second, the third and/or the fourth genetic modification herein. In an embodiment, the first recombinant yeast host cell lacks the second, the third or the fourth genetic modification defined herein. In another embodiment, the first recombinant yeast host cell lacks the second, the third and the fourth genetic modification defined herein. In yet another embodiment, the second recombinant yeast host cell comprises the second, the third or the fourth genetic modifications as defined herein. In yet another embodiment, the second recombinant yeast host cell comprises the second, the third and the fourth genetic modifications as defined herein. In an embodiment, the first recombinant yeast host cell and/or the second recombinant yeast host cell is from the genus Saccharomyces, such as, for example, from the species Saccharomyces cerevisiae.

According to a third aspect, the present disclosure provides a process for promoting ethanolic fermentation, the method comprising fermenting a medium with the first recombinant yeast host cell defined herein or with the cellular population defined herein. In an embodiment, the medium comprises raw starch. In another embodiment, the medium comprises lignocellulose. In another embodiment, the medium is derived from corn. In still another embodiment, the medium is derived from barley, such as, for example, malted barley.

According to a fourth aspect, the present disclosure provides a method of producing an heterologous protease in a first recombinant yeast host cell, the method comprising culturing a first recombinant yeast host cell as defined herein under conditions allowing the expression of the heterologous protease. In an embodiment, the method further comprises introducing a first, second, third and/or fourth genetic modification as defined herein to obtain the first recombinant yeast host cell. Alternatively or in combination, the method can further comprise substantially isolating the heterologous protease from the first recombinant yeast host cell.

According to a fifth aspect, the present disclosure provides a recombinant heterologous protease obtainable by the method described herein. The heterologous protease can be a polypeptide having the amino acid sequence of SEQ ID NO: 2, 6, 8, 10, 12, 14, 30, 36, 38, 40, 42, 52 or 92. The heterologous protease can be a variant having at least 70% identity to the amino acid sequence of SEQ ID NO: 2, 6, 8, 10, 12, 14, 30, 36, 38, 40, 42, 52 or 92 and exhibiting proteolytic activity. The heterologous protease can be a fragment having at least 70% identity to the amino acid sequence of SEQ ID NO: 2, 6, 8, 10, 12, 14, 30, 36, 38, 40, 42, 52 or 92 or the variant described herein and exhibiting proteolytic activity. In an embodiment, the heterologous protease is the polypeptide having the amino acid sequence of SEQ ID NO: 2, 14, 40 or 52. In still another embodiment, the heterologous protease is the variant of the polypeptide having the amino acid sequence of SEQ ID NO: 2, 14, 40 or 52. In still another embodiment, the heterologous protease is the fragment of the polypeptide having the amino acid sequence of SEQ ID NO: 2, 14, 40 or 52. In a further embodiment, the heterologous protease has the amino acid sequence of SEQ ID NO: 2, is the variant of the polypeptide of SEQ ID NO: 2 or is the fragment of the polypeptide of SEQ ID NO: 2. In still another embodiment, the heterologous protease has the amino acid sequence of SEQ ID NO: 14, is the variant of the polypeptide of SEQ ID NO: 14 or is the fragment of the polypeptide of SEQ ID NO: 14. In yet another embodiment, the heterologous protease has the amino acid sequence of SEQ ID NO: 40, is the variant of the polypeptide of SEQ ID NO: 40 or is the fragment of the polypeptide of SEQ ID NO: 40. In a further embodiment, the heterologous protease has the amino acid sequence of SEQ ID NO: 52, is the variant of the polypeptide of SEQ ID NO: 52 or is the fragment of the polypeptide of SEQ ID NO: 52.

According to a sixth aspect, the present disclosure provides a composition comprising an heterologous protease as defined herein. The heterologous protease can be a polypeptide having the amino acid sequence of SEQ ID NO: 2, 6, 8, 10, 12, 14, 30, 36, 38, 40, 42, 52 or 92. The heterologous protease can be a variant having at least 70% identity to the amino acid sequence of SEQ ID NO: 2, 6, 8, 10, 12, 14, 30, 36, 38, 40, 42, 52 or 92 and exhibiting proteolytic activity. The heterologous protease can be a fragment having at least 70% identity to the amino acid sequence of SEQ ID NO: 2, 6, 8, 10, 12, 14, 30, 36, 38, 40, 42, 52 or 92 or the variant described herein and exhibiting proteolytic activity. In an embodiment, the heterologous protease is the polypeptide having the amino acid sequence of SEQ ID NO: 2, 14, 40 or 52. In still another embodiment, the heterologous protease is the variant of the polypeptide having the amino acid sequence of SEQ ID NO: 2, 14, 40 or 52. In still another embodiment, the heterologous protease is the fragment of the polypeptide having the amino acid sequence of SEQ ID NO: 2, 14, 40 or 52. In a further embodiment, the heterologous protease has the amino acid sequence of SEQ ID NO: 2, is the variant of the polypeptide of SEQ ID NO: 2 or is the fragment of the polypeptide of SEQ ID NO: 2. In still another embodiment, the heterologous protease has the amino acid sequence of SEQ ID NO: 14, is the variant of the polypeptide of SEQ ID NO: 14 or is the fragment of the polypeptide of SEQ ID NO: 14. In yet another embodiment, the heterologous protease has the amino acid sequence of SEQ ID NO: 40, is the variant of the polypeptide of SEQ ID NO: 40 or is the fragment of the polypeptide of SEQ ID NO: 40. In a further embodiment, the heterologous protease has the amino acid sequence of SEQ ID NO: 52, is the variant of the polypeptide of SEQ ID NO: 52 or is the fragment of the polypeptide of SEQ ID NO: 52. In an embodiment, the heterologous protease is obtainable/obtained from a first recombinant yeast host cell as defined herein. Alternatively or in combination, the composition can further comprise a glucoamylase as defined herein. further comprising a medium which can, for example comprise raw starch. In an embodiment, the medium is derived from corn or from barley (and, in some instances, can be derived from malted barley).

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 compares the absolute protease activity (using azoalbumin as a substrate) when expressed in an heterologous fashion in Saccharomyces cerevisiae. Results are provided as normalized protease activity in function of the heterologous protease expressed (refer to Table 1 for a description of the proteases used).

FIG. 2 compares the ethanol and glycerol yield of M2390, M10874, M10885, M11589 and M12184 strains during corn fermentation. Results are provided as g of ethanol (first four bars for each strain tested) or glycerol/L (last bar for each strain tested).

FIG. 3 compares the ethanol and glycerol yield of M2390, M10874, M10885, M12982 and M10890 strains during corn fermentation. Results are provided as g of ethanol (first four bars for each strains tested) or glycerol/L (last bar for each strain tested).

FIGS. 4A-4C compare the amino acid sequences of proteases MP818 (SEQ ID NO: 14), MP812 (SEQ ID NO: 2), MP914 (SEQ ID NO: 52) and MP831 (SEQ ID NO: 40). Consensus sequence is provided as SEQ ID NO: 92.

### DETAILED DESCRIPTION

The present disclosure provides recombinant yeast host cell expressing an heterologous proteases for increasing the fermentation rate as well as overall ethanol yield. In some embodiments, the recombinant yeast host cell expressing the heterologous proteases can also decrease glycerol production during fermentation and can even decrease the cost of adding purified enzymes to the fermentation medium.

Proteases are a class of enzymes capable of hydrolyzing polypeptide chains by breaking the peptide bonds linking amino acids. Proteases can release amino acids from the terminal end of a protein (e.g., exopeptidase) or internally (e.g., endopeptidase). There are six categories of proteases which are defined by their mode of action. These include aspartic, glutamic and metallo proteases which activate a water molecule to break the peptide bond as well as serine, threonine and cysteine proteases which create an intermediate product by covalently linking the enzyme to the peptide bond, and then a water molecule is activated to break the bond. Proteases can further be broken down into families, subfamilies and clans. Proteases can also be classified by their optimal pH: neutral, acid, or alkaline. The MEROPS database is dedicated to the classification of known proteases and their function (http://merops.sanger.ac.uk).

### Recombinant Yeast Host Cells

The present disclosure provides a recombinant yeast host cell expressing (and in some embodiments secreting) an heterologous protease. As used in the context of the present disclosure, the “recombinant yeast host cell” includes at least one genetic modification. In the context of the present disclosure, when recombinant yeast host cell is qualified has “having a genetic modification “or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and is the results of in vitro manipulations of the recombinant host cell. When the genetic modification is the addition of an heterologous nucleic acid molecule, such addition can be made once or multiple times at the same or different integration sites. Also, the genetic modification can include introducing one or more nucleic acid molecule which may have been endogenous to the recombinant yeast host cell, provided that this modification be added at a different locus than the endogenous locus. When the genetic modification is the modification of an endogenous nucleic acid molecule, it can be made in one or both copies of the targeted gene.

When expressed in a recombinant yeast host cells, the polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

The present disclosure also provides a method of producing the recombinant yeast host cell by introducing one or more genetic modifications (usually by introducing one or more heterologous nucleic acid molecules) in a yeast cell to provide a recombinant yeast host cell. In an embodiment, an heterologous nucleic acid encoding an heterologous protease is introduced into yeast cell to provide the recombinant yeast host cell. In some embodiments, the method comprises placing the recombinant yeast host cell under conditions so as to favor the expression of the heterologous protease (encoded by an heterologous nucleic acid molecule) by the recombinant yeast host cell.

When an heterologous nucleic acid molecule is present in the recombinant host cell, it can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

In the context of the present disclosure, the recombinant host cell can be a recombinant yeast host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the recombinant yeast host cell is from the following species: Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis.

In some embodiment, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae. In one particular embodiment, the recombinant yeast host cell is Saccharomyces cerevisiae.

In some embodiments, heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous protein from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream. In the context of the present disclosure, it is possible to use a constitutive or an inducible promoter for expressing the heterologous proteins.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from different genus that the host cell.

### First Genetic Modification Allowing the Expression of an Heterologous Protease

As indicated in the Example below, the expression of an heterologous protease in a recombinant yeast host cell increases the fermentation rate, increases ethanol yield and/or decrease glycerol production. The Example below also shows that supplementing the fermentation medium with purified proteases does not further increase the fermentation rate, the ethanol yield or decrease glycerol production. As such, the recombinant yeast host cell of the present disclosure include a genetic modification allowing the expression of one or more heterologous protease. As used in the present disclosure, the term “heterologous protease” refers to a polypeptide which was not natively found in the recombinant yeast host cell or which is expressed at a different locus than the native locus in the recombinant yeast host cell.

The disclosure provides a recombinant yeast host cell comprising a first genetic modification allowing the expressing any heterologous protease, except the one disclosed in Guo et al., 2011. The recombinant yeast host cell of the present disclosure can express one or more heterologous proteases. In an embodiment, the heterologous protease is an aspartic protease or a protease susceptible of having aspartic-like activity. The heterologous protease can be derived from a known protease expressed in a prokaryotic (such as a bacteria) or a eukaryotic cell (such as a yeast, a mold, a plant or an animal).

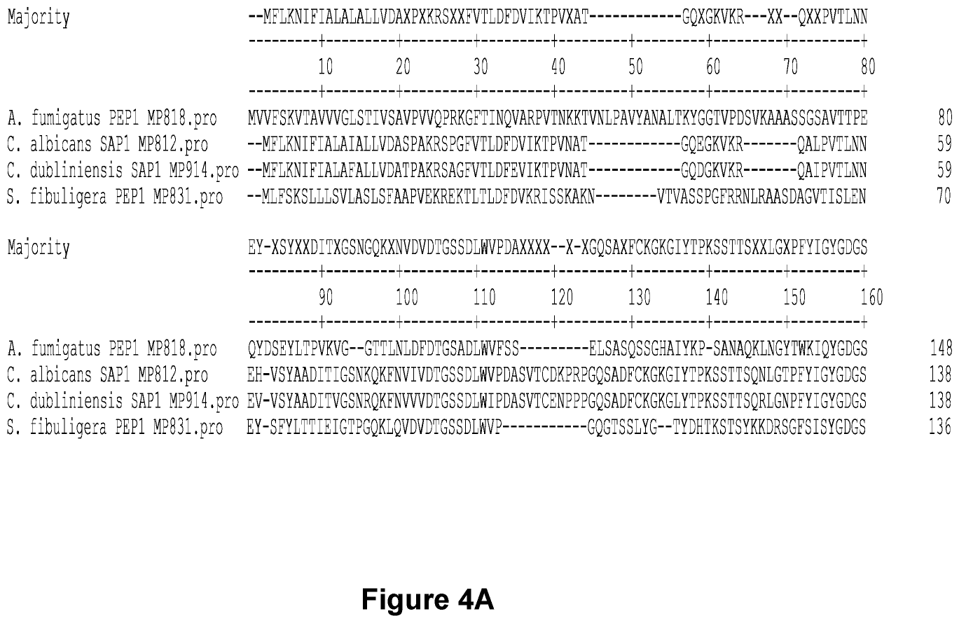

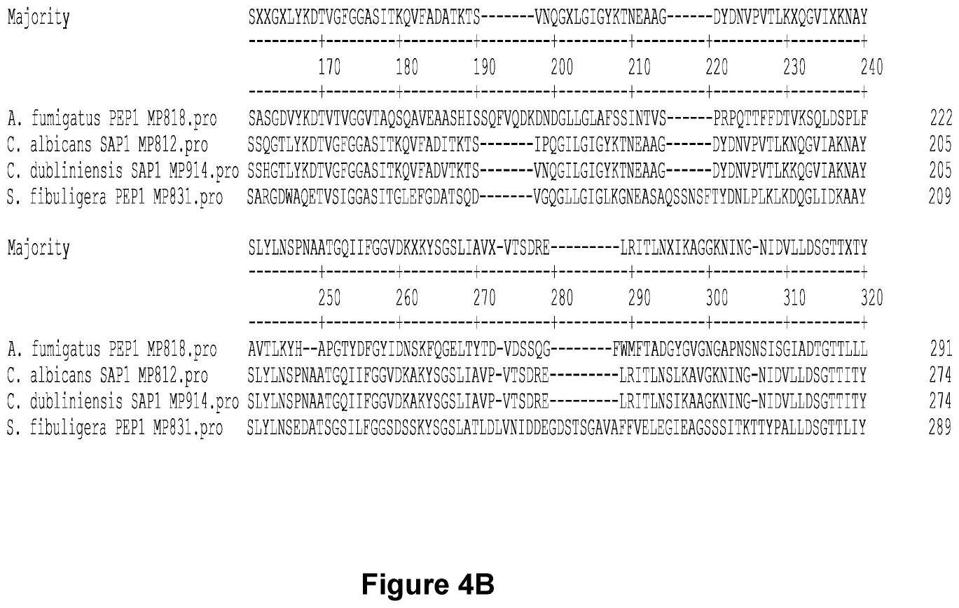

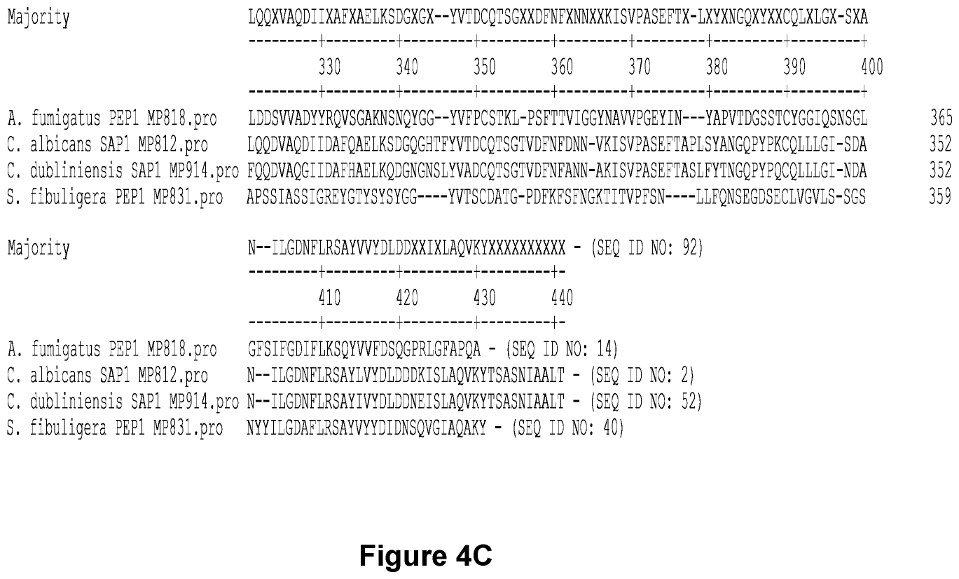

Embodiments of aspartic proteases which can be used according to the present disclosure are shown in FIG. 4. In some embodiments, the protease (its variant or fragment) has any one of the amino acid sequences shown in FIG. 4, including the consensus sequence (SEQ ID NO: 92).

TABLE 1
Characteristics of the proteases presented in FIG. 4.

| | MP # | Gene | MEROPS | Sequence | Peptidase | Active Site |
|---|---|---|---|---|---|---|
| Organism | (SEQ ID #) | Name | ID | Length | Unit | Residues |
| Candida | MP812 | SAP1 | A01.014 | 391 | 43-380 | D82, Y134, |
| albicans | (2) | | | | | D267 |
| Aspergillus | MP818 | PEP1 | A01.026 | 395 | 74-392 | D102, Y144, |
| fumigatus | (14) | | | | | D284 |
| Candida | MP914 | SAP1 | A01.014 | 391 | 43-380 | D82, Y134, |
| dubliniensis | (52) | | | | | D267 |
| Saccharomycopsis | MP831 | PEP1 | unassigned | 390 | 55-389 | D93, Y132, |
| fibuligera | (40) | | | | | D282 |

In an embodiment, the proteases (their variants or fragments) have the consecutive amino acids of the peptidase subunit defined in Table 1. For example, the protease can have residues 43 to 380 of SEQ ID NO: 2, residues 74 to 392 of SEQ ID NO: 14, residues 43 to 380 of SEQ ID NO: 52 or residues 55 to 389 of SEQ ID NO: 40. In still another embodiment, the proteases (their variants or fragments) have the active sites residues of the proteases defined in Table 1. For example, the proteases can have residues corresponding to D82, Y134 and D267 of SEQ ID NO: 2, residues corresponding to D102, Y144 and D284 of SEQ ID NO: 14, residues corresponding to D82, Y134 and D267 of SEQ ID NO: 52 or residues corresponding to D93, Y132 and D282 of SEQ ID NO: 40.

In an embodiment, the heterologous protease can be derived from a fungal organism. For example, the heterologous protease can be derived from the genus Candida, Clavispora, Saccharomyces, Yarrowia, Meyerozyma, Aspergillus or Saccharomycopsis. When the heterologous protease is derived from the genus Candida, it can be derived from the species Candida albicans, Candida dubliniensis or Candida tropicalos. When the heterologous protease is derived from Candida albicans, it can have the amino acid of SEQ ID NO: 2. When the heterologous protease is derived from Candida dubliensis, it can have the amino acid sequence of SEQ ID NO: 52. When the heterologous protease is derived from Candida tropicalis, it can have the amino acid sequence of SEQ ID NO: 38. When the heterologous protease is derived from the genus Clavispora, it can be derived from the species Clavispora lusitaniae. When the heterologous protease is derived from the species Clavispora lusitaniae, it can have the amino acid sequence of SEQ ID NO: 6 or 30. When the heterologous protease is derived from the genus Saccharomyces, it can be derived from the species Saccharomyces cerevisiae. When the heterologous protease is derived from the species Saccharomyces cerevisiae, it can have the amino acid sequence of SEQ ID NO: 8. When the heterologous protease is derived from the genus Yarrowia, it can be derived from the species Yarrowia lipolytica. When the heterologous protease is derived from the species Yarrowia lipolytica, it can have the amino acid sequence of SEQ ID NO: 10. When the heterologous protease is derived from the genus Meyerozyma, it can be derived from the species Meyerozyma guilliermondii. When the heterologous protease is derived from the species Meyerozyma guilliermondii, it can have the amino acid sequence of SEQ ID NO: 12. When the heterologous protease is derived from the genus Aspergillus, it can be derived from the species Aspergillus fumigatus. When the heterologous protease is derived from the species Aspergillus fumigatus, it can have the amino acid sequence of SEQ ID NO: 14. When the heterologous protease is derived from the species Saccharomycopsis, it can be derived from the species Saccharomycopsis fibuligera. When the heterologous protease is derived from the species Saccharomycopsis fibuligera, it can have the amino acid sequence of SEQ ID NO: 40.

In an embodiment, the heterologous protease can be derived from a bacterial organism. For example, the heterologous protease can be derived from the genus Bacillus. When the heterologous protease is derived from the genus Bacillus, it can be derived from the species Bacillus subtilis, it can have the amino acid sequence of SEQ ID NO: 36.

In an embodiment, the heterologous protease can be derived from a plant. For example, the heterologous protease can be derived from the genus Ananas. When the heterologous protease is derived from the genus Ananas, it can be derived from the species Ananas comosus, it can have the amino acid sequence of SEQ ID NO: 42.

In an embodiment, the heterologous protease is a polypeptide having an amino acid sequence of SEQ ID NO: 2, 6, 8, 10, 12, 14, 30, 36, 38, 40, 42 or 52. In still another embodiment, the heterologous protease is a polypeptide having an amino acid sequence of SEQ ID NO: 2, 14, 40 or 52. In yet a further embodiment, the heterologous protease is a polypeptide having the amino acid sequence of SEQ ID NO: 2. In yet a further embodiment, the heterologous protease is a polypeptide having the amino acid sequence of SEQ ID NO: 14. In yet a further embodiment, the heterologous protease is a polypeptide having the amino acid sequence of SEQ ID NO: 40. In yet a further embodiment, the heterologous protease is a polypeptide having the amino acid sequence of SEQ ID NO: 52.

The present disclosure also provides using variants of the polypeptides described herein as the heterologous protease. A “variant” comprises at least one amino acid difference (substitution or addition) when compared to the amino acid sequence of the polypeptides having the amino acid sequence of SEQ ID NO: 2, 6, 8, 10, 12, 14, 30, 36, 38, 40, 42 or 52. The variants do exhibit protease activity, such as aspartic protease activity. Protease activity can be measured by various techniques known in the art, including methods using azoalbumin as a substrate. In an embodiment, the variant exhibits at least 50%, 60%, 70%, 80%, 90%, 95%, 96%, 97%, 98% or 99% proteolytic activity when compared to the proteolytic activity of the polypeptide having the amino acid sequence of SEQ ID NO: 2, 6, 8, 10, 12, 14, 30, 36, 38, 40, 42 or 52. In an embodiment, the variant exhibits at least 70%, 80%, 90%, 95%, 96%, 97%, 98% or 99% identity to the polypeptide having the amino acid sequence of SEQ ID NO: 2, 6, 8, 10, 12, 14, 30, 36, 38, 40, 42 or 52. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variants described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant can be also be a conservative variant or an allelic variant. As used herein, a conservative variant refers to alterations in the amino acid sequence that do not adversely affect the biological functions of the protease (e.g., hydrolysis of proteins). A substitution, insertion or deletion is said to adversely affect the protein when the altered sequence prevents or disrupts a biological function associated with the protease (e.g., the hydrolysis of proteins). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the protease.

In an embodiment, the heterologous protease is a fragment of the polypeptide having the amino acid sequence of SEQ ID NO: 2, 6, 8, 10, 12, 14, 30, 36, 38, 40, 42 or 52. A fragment comprises at least one less amino acid residue when compared to the amino acid sequence of the protease or variant of the protease. The fragment of the protease exhibits proteolytic activity. In an embodiment, the fragment of the protease exhibits at least 50%, 60%, 70%, 80%, 90%, 95%, 96%, 97%, 98% or 99% of the protease activity of the full-length amino acid of of SEQ ID NO: 2, 6, 8, 10, 12, 14, 30, 36, 38, 40, 42 or 52. The protease fragments can also have at least 70%, 80%, 85%, 90%, 95%, 96%, 97%, 98% or 99% identity to the amino acid sequence of of SEQ ID NO: 2, 6, 8, 10, 12, 14, 30, 36, 38, 40, 42 or 52. The fragment can be, for example, a truncation of one or more amino acid residues at the amino-terminus, the carboxy terminus or both terminus of the protease polypeptide or variant. Alternatively or in combination, the fragment can be generated from removing one or more internal amino acid residues. In an embodiment, the alpha-amylase fragment has at least 100, 150, 200, 250, 300, 350 or 400 or more consecutive amino acids of the protease or the variant.

### Second Genetic Modification Allowing the Expression of an Heterologous Glucoamylase

The recombinant yeast host cell having the first genetic modification allowing the expression of the heterologous protease can include one or mode additional genetic modifications.

For example, the recombinant yeast host cell can include a second genetic modification allowing the expression of an heterologous glucoamylase. Alternatively, the recombinant yeast host cell comprising the first genetic modification can be used in combination with another recombinant yeast host cell comprising the second genetic modification allowing the expression of an heterologous glucoamylase. Polypeptides having glucoamylase activity (also referred to as glucoamylases) are exo-acting enzymes capable of terminally hydrolyzing starch to glucose. Glucoamylase activity can be determined by various ways by the person skilled in the art. For example, the glucoamylase activity of a polypeptide can be determined directly by measuring the amount of reducing sugars generated by the polypeptide in an assay in which raw or gelatinized (corn) starch is used as the starting material.

In the context of the present disclosure, the heterologous glucoamylase can be derived from a yeast, for example, from the genus Saccharomycopsis and, in some instances, from the species S. fibuligera. The heterologous glucoamylase can be encoded by the glu0111 gene from S. fibuligera or a glu0111 gene ortholog. An embodiment of glucoamylase polypeptide of the present disclosure is the GLU0111 polypeptide (GenBank Accession Number: CAC83969.1). The GLU0111 polypeptide includes the following amino acids (or correspond to the following amino acids) which are associated with glucoamylase activity and include, but are not limited to amino acids located at positions 41, 237, 470, 473, 479, 485, 487 of SEQ ID NO: 91. The heterologous glucoamylase can be a variant glucoamylase having the amino acids located at positions 41, 237, 470, 473, 479, 485, 487 of SEQ ID NO: 91. The heterologous glucoamylase can be a fragment of SEQ ID NO: 91 having to amino acids located at positions 41, 237, 470, 473, 479, 485, 487 of SEQ ID NO: 91. It is possible to use a polypeptide which does not comprise its endogenous signal sequence. Embodiments of heterologous glucoamylase have been also been described in PCT/US2012/032443 and PCT/US2011/039192.

In the context of the present disclosure, a “glu0111 gene ortholog” is understood to be a gene in a different species that evolved from a common ancestral gene by speciation. In the context of the present disclosure, a glu0111 ortholog retains the same function, e.g. it can act as a glucoamylase. Glu0111 gene orthologs includes but are not limited to, the nucleic acid sequence of GenBank Accession Number XP_003677629.1 (Naumovozyma castellii) XP_003685231.1 (Tetrapisispora phaffii), XP_455264.1 (Kluyveromyces lactis), XP_446481.1 (Candida glabrata), EER33360.1 (Candida tropicalis), EEQ36251.1 (Clavispora lusitaniae), ABN68429.2 (Scheffersomyces stipitis), AAS51695.2 (Eremothecium gossypi), EDK43905.1 (Lodderomyces elongisporus), XP_002555474.1 (Lachancea thermotolerans), EDK37808.2 (Pichia guilliermondii), CAA86282 (Saccharomyces cerevisiae), XP_003680486.1 (Torulaspora delbrueckii), XP_503574.1 (Yarrowia lipolytica), XP_002496552.1 (Zygosaccharomyces rouxii), CAX42655.1 (Candida dubliniensis), XP_002494017.1 (Komagataella pastoris) and AET38805.1 (Eremothecium cymbalariae).

Still in the context of the present disclosure, a variant of the heterologous glucoamylase can be used. A variant comprises at least one amino acid difference (substitution or addition) when compared to the amino acid sequence of the glucoamylase polypeptide of SEQ ID NO: 91. The glucoamylase variants do exhibit glucoamylase activity. In an embodiment, the variant glucoamylase exhibits at least 50%, 60%, 70%, 80%, 90%, 95%, 96%, 97%, 98% or 99% of the glucoamylase activity of the amino acid of SEQ ID NO: 91. The glucoamylase variants also have at least 70%, 80%, 85%, 90%, 95%, 96%, 97%, 98% or 99% identity to the amino acid sequence of SEQ ID NO: 91. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant glucoamylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant glucoamylase can also be a conservative variant or an allelic variant. As used herein, a conservative variant refers to alterations in the amino acid sequence that do not adversely affect the biological functions of the glucoamylase. A substitution, insertion or deletion is said to adversely affect the protein when the altered sequence prevents or disrupts a biological function associated with the glucoamylase (e.g., the hydrolysis of starch into glucose). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the glucoamylase.

The present disclosure also provides expressing fragments of the glucoamylases polypeptides and glucoamylase variants described herein. A fragment comprises at least one less amino acid residue when compared to the amino acid sequence of the glucoamylase polypeptide or variant and still possess the enzymatic activity of the full-length glucoamylase. In an embodiment, the glucoamylase fragment exhibits at least 50%, 60%, 70%, 80%, 90%, 95%, 96%, 97%, 98% or 99% of the full-length glucoamylase of the amino acid of SEQ ID NO: 91. The glucoamylase fragments can also have at least 70%, 80%, 85%, 90%, 95%, 96%, 97%, 98% or 99% identity to the amino acid sequence of SEQ ID NO: 91. The fragment can be, for example, a truncation of one or more amino acid residues at the amino-terminus, the carboxy terminus or both termini of the glucoamylase polypeptide or variant. Alternatively or in combination, the fragment can be generated from removing one or more internal amino acid residues. In an embodiment, the glucoamylase fragment has at least 100, 150, 200, 250, 300, 350, 400, 450, 500 or more consecutive amino acids of the glucoamylase polypeptide or the variant.

### Third Genetic Modification for Reducing Glycerol Levels

The recombinant host cell comprising the first genetic modification (and optionally the second genetic modification) can also include a third genetic modification for reducing the production of one or more native enzymes that function to produce glycerol or regulate glycerol synthesis. Alternatively, the recombinant yeast host cell comprising the first genetic modification (and optionally the second and/or third genetic modification) can be used in combination with another recombinant yeast host cell comprising the third genetic modification for reducing the production of one or more native enzymes that function to produce glycerol or regulate glycerol synthesis (and optionally the second genetic modification).

As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding strain which does not bear the third genetic modification. In some instances, the third genetic modification reduces but still allows the production of one or more native polypeptides that function to produce glycerol or regulate glycerol synthesis. In other instances, the third genetic modification inhibits the production of one or more native enzymes that function to produce glycerol or regulate glycerol synthesis. In some embodiments, the recombinant host cells bear a plurality of third genetic modifications, wherein at least one reduces the production of one or more native polypeptides and at least another inhibits the production of one or more native polypeptides.

As used in the context of the present disclosure, the expression “native polypeptides that function to produce glycerol or regulate glycerol synthesis” refers to polypeptides which are endogenously found in the recombinant yeast host cell. Native enzymes that function to produce glycerol include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). Native enzymes that function to regulating glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol synthesis. In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide), the gpp2 gene (encoding the GPP2 polypeptide), the fps1 gene (encoding the FPS1 polypeptide) or orthologs thereof. In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide), the gpp2 gene (encoding the GPP2 polypeptide), the fps1 gene (encoding the FPS1 polypeptide) or orthologs thereof. In still another embodiment, the recombinant yeast host cell bears a genetic modification in each of the gpd1 gene (encoding the GPD1 polypeptide), the gpd2 gene (encoding the GPD2 polypeptide) and the fps1 gene (encoding the FPS1 polypeptide) or orthologs thereof. Examples of recombinant yeast host cells bearing such genetic modification(s) leading to the reduction in the production of one or more native enzymes that function to produce glycerol or regulating glycerol synthesis are described in WO 2012/138942. Preferably, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene, the gpd2 gene and the fps1 gene resulting is a recombinant yeast host cell being knock-out for the gpd1 gene, the gpd2 gene and the fps1 gene. In still another embodiment (in combination or alternative to the “first” genetic modification described above), the recombinant yeast host cell can have a genetic modification in the stl1 gene (e.g., a duplication for example) for increasing the expression of the STL1 polypeptide. In an embodiment, the recombinant yeast host cell can have a genetic modification in the gpd2 genes.

### Fourth Genetic Modification for Maintaining or Increasing Formate Levels

The recombinant host cell comprising the first genetic modification (and optionally the second and/or third genetic modification) can also include a fourth genetic modification for reducing the production of one or more native enzymes that function to catabolize formate. Alternatively, the recombinant yeast host cell comprising the first genetic modification (and optionally the second, third and/or fourth genetic modification) can be used in combination with another recombinant yeast host cell comprising the fourth genetic modification for reducing the production of one or more native enzymes that function to catabolize formate (and optionally the second and/or third genetic modification).

As used in the context of the present disclosure, the expression “for reducing the production of one or more native enzymes that function to catabolize formate”. As used in the context of the present disclosure, the expression “native polypeptides that function to catabolize formate” refers to polypeptides which are endogenously found in the recombinant host cell. Native enzymes that function to catabolize formate include, but are not limited to, the FDH1 and the FDH2 polypeptides (also referred to as FDH1 and FDH2 respectively). In an embodiment, the recombinant yeast host cell bears a genetic modification in at least one of the fdh1 gene (encoding the FDH1 polypeptide), the fdh2 gene (encoding the FDH2 polypeptide) or orthologs thereof. In another embodiment, the recombinant yeast host cell bears genetic modifications in both the fdh1 gene (encoding the FDH1 polypeptide) and the fdh2 gene (encoding the FDH2 polypeptide) or orthologs thereof. Examples of recombinant yeast host cells bearing such genetic modification(s) leading to the reduction in the production of one or more native enzymes that function to catabolize formate are described in WO 2012/138942. Preferably, the recombinant yeast host cell has genetic modifications (such as a genetic deletion or insertion) in the fdh1 gene and in the fdh2 gene which would cause the host cell to have knocked-out fdh1 and fdh2 genes.

In some embodiments, the recombinant yeast host cell can include a further genetic modification for increasing the production of an heterologous enzyme that function to anabolize (form) formate. As used in the context of the present disclosure, “an heterologous enzyme that function to anabolize formate” refers to polypeptides which may or may not be endogenously found in the recombinant yeast host cell and that are purposefully introduced into the recombinant yeast host cells. In some embodiments, the heterologous enzyme that function to anabolize formate is an heterologous pyruvate formate lyase (PFL), an heterologous acetaldehyde dehydrogenases, an heterologous alcohol dehydrogenases, and/or and heterologous bifunctional acetylaldehyde/alcohol dehydrogenases (AADH) such as those described in U.S. Pat. No. 8,956,851 and PCT/US2014/051355. More specifically, PFL and AADH enzymes for use in the recombinant yeast host cells can come from a bacterial or eukaryotic source. Heterologous PFL of the present disclosure include, but are not limited to, the PFLA polypeptide, a polypeptide encoded by a pfla gene ortholog, the PFLB polyeptide or a polypeptide encoded by a pflb gene ortholog. Heterologous AADHs of the present disclosure include, but are not limited to, the ADHE polypeptides or a polypeptide encoded by an adhe gene ortholog. In an embodiment, the recombinant yeast host cell of the present disclosure comprises at least one of the following heterologous enzymes that function to anabolize formate: the PFLA polypeptide, the PFLB polypeptide and/or the ADHE polypeptide. In an embodiment, the recombinant yeast host cell of the present disclosure comprises at least two of the following heterologous enzymes that function to anabolize formate: the PFLA polypeptide, the PFLB polypeptide and/or the ADHE polypeptide. In another embodiment, the recombinant yeast host cell of the present disclosure comprises the following heterologous enzymes that function to anabolize formate: the PFLA polypeptide, the PFLB polypeptide and the ADHE polypeptide.

### Additional Genetic Modifications

The recombinant host cell can be further genetically modified to allow for the production of additional heterologous polypeptides. In an embodiment, the recombinant yeast host cell can be used for the production of an enzyme, and especially an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases (other than those described above), cellulases, hemicellulases, cellulolytic and amylolytic accessory enzymes, inulinases, levanases, trehalases, pectinases, and pentose sugar utilizing enzymes.

The additional heterologous polypeptide can be an “amylolytic enzyme”, an enzyme involved in amylase digestion, metabolism and/or hydrolysis. The term “amylase” refers to an enzyme that breaks starch down into sugar. All amylases are glycoside hydrolases and act on α-1,4-glycosidic bonds. Some amylases, such as γ-amylase (glucoamylase), also act on α-1,6-glycosidic bonds. Amylase enzymes include α-amylase (EC 3.2.1.1), β-amylase (EC 3.2.1.2), and γ-amylase (EC 3.2.1.3). The α-amylases are calcium metalloenzymes, unable to function in the absence of calcium. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster-acting than β-amylase. Another form of amylase, β-amylase is also synthesized by bacteria, fungi, and plants. Working from the non-reducing end, β-amylase catalyzes the hydrolysis of the second α-1,4 glycosidic bond, cleaving off two glucose units (maltose) at a time. Another amylolytic enzyme is α-glucosidase that acts on maltose and other short malto-oligosaccharides produced by α-, β-, and γ-amylases, converting them to glucose. Another amylolytic enzyme is pullulanase. Pullulanase is a specific kind of glucanase, an amylolytic exoenzyme, that degrades pullulan. Pullulan is regarded as a chain of maltotriose units linked by alpha-1,6-glycosidic bonds. Pullulanase (EC 3.2.1.41) is also known as pullulan-6-glucanohydrolase (debranching enzyme). Another amylolytic enzyme, isopullulanase, hydrolyses pullulan to isopanose (6-alpha-maltosylglucose). Isopullulanase (EC 3.2.1.57) is also known as pullulan 4-glucanohydrolase. An “amylase” can be any enzyme involved in amylase digestion, metabolism and/or hydrolysis, including α-amylase, β-amylase, glucoamylase, pullulanase, isopullulanase, and alpha-glucosidase.

The additional heterologous polypeptide can be a “cellulolytic enzyme”, an enzyme involved in cellulose digestion, metabolism and/or hydrolysis. The term “cellulase” refers to a class of enzymes that catalyze cellulolysis (i.e. the hydrolysis) of cellulose. Several different kinds of cellulases are known, which differ structurally and mechanistically. There are general types of cellulases based on the type of reaction catalyzed: endocellulase breaks internal bonds to disrupt the crystalline structure of cellulose and expose individual cellulose polysaccharide chains; exocellulase cleaves 2-4 units from the ends of the exposed chains produced by endocellulase, resulting in the tetrasaccharides or disaccharide such as cellobiose. There are two main types of exocellulases (or cellobiohydrolases, abbreviate CBH)—one type working processively from the reducing end, and one type working processively from the non-reducing end of cellulose; cellobiase or beta-glucosidase hydrolyses the exocellulase product into individual monosaccharides; oxidative cellulases that depolymerize cellulose by radical reactions, as for instance cellobiose dehydrogenase (acceptor); cellulose phosphorylases that depolymerize cellulose using phosphates instead of water. In the most familiar case of cellulase activity, the enzyme complex breaks down cellulose to beta-glucose. A “cellulase” can be any enzyme involved in cellulose digestion, metabolism and/or hydrolysis, including an endoglucanase, glucosidase, cellobiohydrolase, xylanase, glucanase, xylosidase, xylan esterase, arabinofuranosidase, galactosidase, cellobiose phosphorylase, cellodextrin phosphorylase, mannanase, mannosidase, xyloglucanase, endoxylanase, glucuronidase, acetylxylanesterase, arabinofuranohydrolase, swollenin, glucuronyl esterase, expansin, pectinase, and feruoyl esterase protein.

The additional heterologous polypeptide can have “hemicellulolytic activity”, an enzyme involved in hemicellulose digestion, metabolism and/or hydrolysis. The term “hemicellulase” refers to a class of enzymes that catalyze the hydrolysis of cellulose. Several different kinds of enzymes are known to have hemicellulolytic activity including, but not limited to, xylanases and mannanases.

The additional heterologous polypeptide can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous protein can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein. A “pentose sugar utilizing enzyme” can be any enzyme involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase.

The additional heterologous polypeptide can have “mannanic activity”, an enzyme having the is ability to hydrolyze the terminal, non-reducing β-D-mannose residues in β-D-mannosides. Mannanases are capable of breaking down hemicellulose, one of the major components of plant cell walls. Xylanases include those enzymes that correspond to Enzyme Commission Number 3.2.25.

The additional heterologous polypeptide can be a “pectinase”, an enzyme, such as pectolyase, pectozyme and polygalacturonase, commonly referred to in brewing as pectic enzymes. These enzymes break down pectin, a polysaccharide substrate that is found in the cell walls of plants.

The additional heterologous polypeptide can have “phytolytic activity”, an enzyme catalyzing the conversion of phytic acid into inorganic phosphorus. Phytases (EC 3.2.3) can be belong to the histidine acid phosphatases, β-propeller phytases, purple acid phosphastases or protein tyrosine phosphatase-like phytases family.

### Cellular Populations

The present disclosure also provides cellular population comprising the recombinant yeast host cell comprising the first genetic modification. In an embodiment, the cellular population comprises or consists essentially of one or more of the recombinant yeast host cell comprising the first genetic modification (and in an embodiment, lacking the second, the third, the fourth and/or a further genetic modification). In some embodiments, the cellular population can also include non-genetically modified fermenting yeasts.

In yet another embodiment, the cellular population comprises a first recombinant yeast host cell (comprising at least the first genetic modification) and a second recombinant yeast host cell (comprising at least the second, third and/or fourth genetic modification) and optionally non-genetically-modified fermenting yeasts. In still another embodiment, the cellular population comprises a first recombinant yeast host cell (comprising the first genetic modification) and a second recombinant yeast host cell (comprising at least the second, third or fourth genetic modification) and optionally non-genetically-modified fermenting yeasts. In yet a further embodiment, the cellular population comprises a first recombinant yeast host cell (comprising the first genetic modification) and a second recombinant yeast host cell (comprising at least two of the second, third or fourth genetic modification) and optionally non-genetically-modified fermenting yeasts. In still another embodiment, the cellular population comprises a first recombinant yeast host cell (comprising the first genetic modification) and a second recombinant yeast host cell (comprising the second and third genetic modifications) and optionally non-genetically-modified fermenting yeasts. In another embodiment, the cellular population comprises a first recombinant yeast host cell (comprising the first genetic modification) and a second recombinant yeast host cell (comprising the second, third and fourth genetic modifications) and optionally non-genetically-modified fermenting yeasts.

The cellular population can be provided in a liquid or solid form (e.g., in some embodiments in a freeze-dried form or as a cream yeast). The cellular population can be provided as a single unit comprising both the first recombinant yeast host cell and the second recombinant yeast host cell. Alternatively, the cellular population can be provided in two units each comprising the first recombinant yeast host cell and the second recombinant yeast host cell.

The recombinant yeast host cells of the cellular population can be from the same or from different genus. In an embodiment, the recombinant yeast host cells of the cellular population can be from the same or different species. In still another embodiment, the recombinant yeast host cells of the cellular population are from the genus Saccharomyces and, in further embodiment, from the species Saccharomyces cerevisiae.

### Process for Using the Recombination Yeast Host Cells and the Cellular Populations and Associated Compositions

As indicated herein, the use of a recombinant yeast host cell comprising the first genetic modification during allows to increase the fermentation rate and the ethanol yield when compared to a corresponding fermentation made by yeast cells lacking the first genetic modification.

Embodiments in which the cellular population does not include a recombinant yeast host cell comprising the second, third and/or fourth genetic modifications as described herein are especially useful for the production of distilled spirits. In such embodiments, the first recombinant yeast host cell (comprising the first genetic modification) or a cellular population comprising same can be used to ferment a medium to make ethanol. The distilled spirits fermentation medium can comprise, for example, a grain (barley, rye, corn, sorghum, wheat, rice, millet, buckwheat), a fruit (grape, apple, pear, plum, apricots, quinces, pineapple, juniper berry, bananas, plantain, gougi, coconut, ginger, pomace, cashew) and/or a vegetable (cassava, potato, sugar cane, molasses, agave). The distilled spirit can be, but is not limited to scotch whisky, rye whisky, vodka, brandy, cognac, vermouth, armagnac, calvados, cider, rhum. After fermentation, the fermentation medium can be distilled into the distilled spirit.

Embodiments in which the cellular population comprises recombinant yeast host cells comprising the first, second, third and/or fourth genetic modifications as well as cellular populations comprising same can be useful for the production of ethanol for biofuel applications. In some embodiment, a cellular population comprising the first recombinant yeast host cell comprising the first genetic modification and the second recombinant yeast host cell comprising the second, third and fourth genetic modifications can be used for the production of ethanol for biofuel applications. Broadly, the process comprises combining a substrate to be hydrolyzed (optionally included in a fermentation medium) with the recombinant host cells of the cellular populations. In an embodiment, the substrate to be hydrolyzed is a lignocellulosic biomass and, in some embodiments, it comprises starch (in a gelatinized or raw form). In some embodiments, the use of recombinant host cells avoids the need of adding additional external source of purified enzymes during fermentation to allow the breakdown of starch.

The production of ethanol can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, when a thermotolerant yeast cell is used in the process, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

### Heterologous Protease

The present disclosure also provides the heterologous proteases disclosed herein expressed in a recombinant form. The heterologous proteases can be obtained by recombinant production in the first recombinant yeast host cell. In some embodiments, the method comprises culturing the recombinant yeast host cell of the present disclosure under conditions so as to allow the expression of the heterologous protease. The culturing step can be a continuous culture, a batch culture or a fed-batch culture. For example, the culture medium can comprise a carbon source (such as, for example, molasses, sucrose, glucose, dextrose syrup, ethanol and/or corn steep liquor), a nitrogen source (such as, for example, ammonia) and a phosphorous source (such as, for example, phosphoric acid). The method can further comprises, for example, a step of introducing the first, second, third and/or fourth genetic modification as described herein prior to the culturing step. The method can also comprises, in some instances, removing at least one component for the medium or substantially isolating the heterologous protease from the medium. The medium component that can be removed include, without limitation, water, amino acids, peptides and proteins, nucleic acid residues and nucleic acid molecules, cellular debris, fermentation products, etc. In an embodiment, the method can also comprise substantially isolating the cultured yeast recombinant host cells (e.g., the biomass) from the components of the culture medium. As used in the context of the present disclosure, the expression “substantially isolating” refers to the removal of the majority of the components of the culture medium from the cultured recombinant yeast host cells. In order to do so, the cultured recombinant yeast host cells can be centrifuged (and the resulting cellular pellet comprising the propagated recombinant yeast host cells can optionally be washed), filtered and/or dried (optionally using a vacuum-drying technique).

The heterologous proteases can be provided in an isolated form or can be provided as a composition. The composition can optionally include a component from a medium (which can comprise raw starch, for example, derived from corn and/or barley) and/or a glucoamylase as described herein.

The present invention will be more readily understood by referring to the following examples which are given to illustrate the invention rather than to limit its scope.

### EXAMPLE

TABLE 2
Description of the enzymes used in the Example.

| Designation | Description |
|---|---|
| | 1) Organism |
| | 2) Merops ID |
| | 3) EC# |
| | 4) Accession # |
| | 5) Alternative name |
| | 6) Type |
| | 7) SEQ ID NO |
| MP812 | 1) Candida albicans |
| | 2) A01.014 |
| | 3) 3.4.23.24 |
| | 4) C4YSF6 |
| | 5) SAP1, candidapepsin-1 |
| | 6) Aspartic |
| | 7) SEQ ID NO: 2 |
| MP813 | 1) Aspergillus fumigatus |
| | 2) A01.018 |
| | 3) Unknown |
| | 4) O42630 |
| | 5) pep2 |
| | 6) Unknown |
| | 7) SEQ ID NO: 4 |
| MP814 | 1) Clavispora lusitaniae |
| | 2) A01.018 |
| | 3) Unknown |
| | 4) C4Y7E6 |
| | 5) Saccharopepsin |
| | 6) Aspartic |
| | 7) SEQ ID NO: 6 |
| MP815 | 1) Saccharomyces cerevisiae |
| | 2) A01.018 |
| | 3) 3.4.23.25 |
| | 4) P07267 |
| | 5) saccharopepsin, PEP4 |
| | 6) Aspartic |
| | 7) SEQ ID NO: 8 |
| MP816 | 1) Yarrowia lipolytica |
| | 2) A01.018 |
| | 3) Q6C080 |
| | 4) Saccharopepsin |
| | 5) None |
| | 6) Aspartic |
| | 7) SEQ ID NO: 10 |
| MP817 | 1) Meyerozyma guilliermondii |
| | 2) A01.018 |
| | 3) |
| | 4) A5DLJ4 |
| | 5) PGUG_04145 |
| | 6) Putative aspartic |
| | 7) SEQ ID NO: 12 |
| MP818 | 1) Aspergillus fumigatus |
| | 2) A01.026 |
| | 3) 3.4.23.18 |
| | 4) P41748 |
| | 5) pep1 |
| | 6) Aspartic |
| | 7) SEQ ID NO: 14 |
| MP819 | 1) Saccharomyces cerevisiae |
| | 2) A01.030 |
| | 3) 3.4.23.41 |
| | 4) P32329 |
| | 5) YPS1 |
| | 6) Aspartic |
| | 7) SEQ ID NO: 16 |
| MP820 | 1) Yarrowia lipolytica |
| | 2) A01.030 |
| | 3) Unknown |
| | 4) Q6CAN1 |
| | 5) YALI0D01331p |
| | 6) Aspartic |
| | 7) SEQ ID NO: 18 |
| MP821 | 1) Meyerozyma guilliermondii |
| | 2) A01.030 |
| | 3) Unknown |
| | 4) A5DF74 |
| | 5) PGUG_01925 |
| | 6) Putative aspartic |
| | 7) SEQ ID NO: 20 |
| MP822 | 1) Saccharomyces cerevisiae |
| | 2) A01.035 |
| | 3) Unknown |
| | 4) Q12303 |
| | 5) YPS3 |
| | 6) Unknown |
| | 7) SEQ ID NO: 22 |
| MP823 | 1) Candida tropicalis |
| | 2) A01.037 |
| | 3) 3.4.23.24 |
| | 4) Q00663 |
| | 5) SAPT1 |
| | 6) Aspartic |
| | 7) SEQ ID NO: 24 |
| MP824 | 1) Clavispora lusitaniae |
| | 2) A01.038 |
| | 3) Unknown |
| | 4) C4Y9C0 |
| | 5) Candiparapsin |
| | 6) Unknown |
| | 7) SEQ ID NO: 26 |
| MP825 | 1) Meyerozyma guilliermondii |
| | 2) A01.038 |
| | 3) |
| | 4) A5DHF0 |
| | 5) PGUG_02701 |
| | 6) Putative aspartic |
| | 7) SEQ ID NO: 28 |
| MP826 | 1) Clavispora lusitaniae |
| | 2) A01.067 |
| | 3) Unknown |
| | 4) C4Y3R6 |
| | 5) candiapepsin SAP9 |
| | 6) Unknown |
| | 7) SEQ ID NO: 30 |
| MP827 | 1) Candida albicans |
| | 2) A01.067 |
| | 3) 3.4.23.24 |
| | 4) O42779 |
| | 5) SAP9 |
| | 6) Aspartic |
| | 7) SEQ ID NO: 32 |
| MP828 | 1) Meyerozyma guilliermondii |
| | 2) A01.067 |
| | 3) Unknown |
| | 4) A5D9Q1 |
| | 5) PGUG_00002 |
| | 6) Putative aspartic |
| | 7) SEQ ID NO: 34 |
| MP829 | 1) Bacillus subtilis |
| | 2) M04.014 |
| | 3) 3.4.24.28 |
| | 4) A0A0A0TWG6 |
| | 5) nprE |
| | 6) Metalloprotease |
| | 7) SEQ ID NO: 36 |
| MP830 | 1) Candida tropicalis |
| | 2) Unassigned |
| | 3) Unknown |
| | 4) Q9Y776 |
| | 5) SAPT4 |
| | 6) Aspartic |
| | 7) SEQ ID NO: 38 |
| MP831 | 1) Saccharomycopsis fibuligera |
| | 2) Unassigned |
| | 3) 3.4.23.— |
| | 4) P22929 |
| | 5) PEP1 |
| | 6) Aspartic |
| | 7) SEQ ID NO: 40 |
| MP832 | 1) Ananas comosus |
| | 2) C01.028 |
| | 3) 3.4.22.33 |
| | 4) O23791 |
| | 5) Unknown |
| | 6) Unknown |
| | 7) SEQ ID NO: 42 |
| MP833 | 1) Ananas comosus |
| | 2) C01.005 |
| | 3) 3.4.22.32 |
| | 4) P14518 |
| | 5) Unknown |
| | 6) Unknown |
| | 7) SEQ ID NO: 44 |
| MP860 | 1) zea mays Vignain like |
| | 2) C1A |
| | 3) |
| | 4) B6TYM9 |
| | 5) vignain like |
| | 6) Unknown |
| | 7) SEQ ID NO: 46 |
| MP861 | 1) zea mays cysteine protease |
| | 2) 1C1A |
| | 3) |
| | 4) B4FS90 |
| | 5) cysteine protease 1 |
| | 6) Unknown |
| | 7) SEQ ID NO: 48 |
| MP862 | 1) zea mays cysteine protease 1(2) |
| | 2) |
| | 3) |
| | 4) B6T669 |
| | 5) cysteine protease 1 |
| | 6) Unknown |
| | 7) SEQ ID NO: 50 |
| MP914 | 1) Candida dubliniensis |
| | 2) A01.014 |
| | 3) 3.4.23.24 |
| | 4) B9WJ11 |
| | 5) SAP1 |
| | 6) Aspartic |
| | 7) SEQ ID NO: 52 |
| MP915 | 1) Candida orthopsilosis |
| | 2) A01.014 |
| | 3) 3.4.23.24 |
| | 4) H8X9C8 |
| | 5) CORT_0F03710 |
| | 6) Aspartic |
| | 7) SEQ ID NO: 54 |
| MP916 | 1) Meyerozyma guilliermondii |
| | 2) Unassigned |
| | 3) 3.4.23.24 |
| | 4) A5DL07 |
| | 5) PGUG_03958 |
| | 6) Aspartic |
| | 7) SEQ ID NO: 56 |
| MP917 | 1) Scheffersomyces stipites |
| | 2) Unassigned |
| | 3) 3.4.23.24 |
| | 4) A3LZH2 |
| | 5) PICST_63754 |
| | 6) Aspartic |
| | 7) SEQ ID NO: 58 |
| MP918 | 1) Lodderomyces elongisporus |
| | 2) A01.038 |
| | 3) 3.4.23.24 |
| | 4) A5DXL7 |
| | 5) candidapepsin-1 |
| | 6) Aspartic |
| | 7) SEQ ID NO: 60 |
| MP919 | 1) Candida albicans |
| | 2) A01.060 |
| | 3) 3.4.23.24 |
| | 4) P0DJ06 |
| | 5) SAP2 |
| | 6) Aspartic |
| | 7) SEQ ID NO: 62 |
| MP920 | 1) Candida albicans SC5314 |
| | 2) A01.061 |
| | 3) 3.4.23.24 |
| | 4) P0CY29 |
| | 5) SAP3 |
| | 6) Aspartic |
| | 7) SEQ ID NO: 64 |
| MP921 | 1) Candida dubliniensis CD36 |
| | 2) A01.061 |
| | 3) 3.4.23.24 |
| | 4) B9WEB2 |
| | 5) SAP3 |
| | 6) Aspartic |
| | 7) SEQ ID NO: 66 |
| MP922 | 1) Neurospora tetrasperma |
| | 2) A01.UPA |
| | 3) Unknown |
| | 4) F8MN20 |
| | 5) NEUTE1DRAFT_100918 |
| | 6) pepsin-like proteinases |
| | 7) SEQ ID NO: 68 |
| MP923 | 1) Podospora anserine |
| | 2) Unknown |
| | 3) A01.UPA |
| | 4) B2AWU0 |
| | 5) PODANS_7_8310 |
| | 6) aspartic acid protease |
| | 7) SEQ ID NO: 70 |
| MP924 | 1) Grossmannia clavigera |
| | 2) Unknown |
| | 3) A01.UPA |
| | 4) F0XHL4 |
| | 5) CMQ_2598 |
| | 6) aspartic acid protease |
| | 7) SEQ ID NO: 72 |
| MP925 | 1) Chaetomium thermophilum |
| | 2) Unknown |
| | 3) A01.UPA |
| | 4) G0S4R8 |
| | 5) CTHT_0023290 |
| | 6) aspartic acid protease |
| | 7) SEQ ID NO: 74 |
| MP926 | 1) Myceliophthora thermophila ATCC 42464] |
| | 2) Unknown |
| | 3) A01.UPA |
| | 4) G2QBW3 |
| | 5) MYCTH_2305028 |
| | 6) pepsin like protease |
| | 7) SEQ ID NO: 76 |
| MP927 | 1) Magnaporthe oryzae 70-15 |
| | 2) Unknown |
| | 3) A01.UPA |
| | 4) G4N837 |
| | 5) candidapepsin-3 |
| | 6) pepsin-like proteinases |
| | 7) SEQ ID NO: 78 |
| MP928 | 1) Kluveromyces lactis |
| | 2) Unknown |
| | 3) A01.030 |
| | 4) Q6CPL3 |
| | 5) KLLA0_E04049g |
| | 6) pepsin-like proteinases |
| | 7) SEQ ID NO: 80 |
| MP929 | 1) Ashbya gossypii ATCC 10895 |
| | 2) Unknown |
| | 3) A01.035 |
| | 4) Q750Y1 |
| | 5) AGOS_AGL192W |
| | 6) pepsin-like proteinases |
| | 7) SEQ ID NO: 82 |
| MP930 | 1) Thielavia terrestris NRRL 8126 |
| | 2) Unknown |
| | 3) A01.UPA |
| | 4) G2RAU9 |
| | 5) THITE_2155501 |
| | 6) pepsin like protease |
| | 7) SEQ ID NO: 84 |
| MP931 | 1) Neurospora crassa |
| | 2) Unknown |
| | 3) A01.015 |
| | 4) Q7RZM6 |
| | 5) NCU00338 |
| | 6) Unknown |
| | 7) SEQ ID NO: 86 |
| MP932 | 1) Aspergillus niger |
| | 2) Unknown |
| | 3) A01.UPA |
| | 4) E2PT33 |
| | 5) An18g01320 |
| | 6) Unknown |
| | 7) SEQ ID NO: 88 |
| MP933 | 1) Bacillus amyloliquefaciens |
| | 2) Unknown |
| | 3) M04.014 |
| | 4) E1UT71 |
| | 5) Unknown |
| | 6) nprE |
| | 7) SEQ ID NO: 90 |

TABLE 3
Description of the S. cerevisiae strains presented in the Example.

| | | Other transgenes | Genes |
|---|---|---|---|
| Designation | Protease expressed | expressed | inactivated |
| M2390 (wild-type, | None | None | None |
| control) | | | |
| M10874 | Gene encoding Candida | None | Δfcy1 |
| | albicans SAP1 | | |
| | (UniProtKB Accession | | |
| | C4YSF6) (MP812) | | |
| M10877 | Gene encoding | None | Δfcy1 |
| | Clavispora lusitaniae | | |
| | Saccharopepsin | | |
| | (UniProtKB Accession | | |
| | C4Y7E6) (MP814) | | |
| M10885 | Gene encoding | None | Δfcy1 |
| | Aspergillus fumigatus | | |
| | PEP1 (UniProtKB | | |
| | Accession P41748) | | |
| | (MP818) | | |
| M10890 | Gene encoding | None | Δfcy1 |
| | Saccharomycopsis | | |
| | fibuligera PEP1 | | |
| | (UniProtKB Accession | | |
| | P22929) (MP831) | | |
| M12982 | Gene encoding Candida | None | Δfcy1 |
| | dubliniensis SAP1 | | |
| | (UniProtKB Accession | | |
| | B9WJ11) (MP914) | | |
| M11259 | Gene encoding Candida | None | None |
| | albicans SAP1 | | |
| | (UniProtKB Accession | | |
| | C4YSF6) expressed on | | |
| | plasmid (MP812) | | |
| M11260 | Gene encoding | None | None |
| | Aspergillus fumigatus | | |
| | PEP1 (UniProtKB | | |
| | Accession P41748) | | |
| | expressed on plasmid | | |
| | (MP818) | | |
| M11262 | Gene encoding | None | None |
| | Clavispora lusitaniae | | |
| | Saccharopepsin | | |
| | (UniProtKB Accession | | |
| | C4Y7E6) expressed on | | |
| | plasmid (MP814) | | |
| M12184 | Gene encoding Candida | Saccharomycopsis | Δgpd2 |
| | albicans SAP1 | fibuligera glu0111 | Δfdh1 |
| | (UniProtKB Accession | (GeneBank Accession | Δfdh2 |
| | C4YSF6) (MP812) | CAC83969.1) | Δfcy1 |
| | | Gene encoding the PFLA | |
| | | polypeptide (UniProtKB | |
| | | Accession A1A239) | |
| | | Gene encoding the PFLB | |
| | | polypeptide (UniProtKB | |
| | | Accession A1A240) | |
| | | Gene encoding the | |
| | | ADHE polypeptide | |
| | | (UniProtKB Accession | |
| | | A1A067) | |
| | | Gene encoding | |
| | | Saccharomyces | |
| | | cerevisiae STL1 | |
| | | (GeneBank Accession | |
| | | NP_010825 | |
| M12106 | Gene encoding | Gene encoding | Δgpd2 |
| | Aspergillus fumigatus | Saccharomycopsis | Δfdh1 |
| | PEP1 (UniProtKB | fibuligera glu0111 | Δfdh2 |
| | Accession P41748) | (GeneBank Accession | Δfcy1 |
| | (MP818) | CAC83969.1) | |
| | | Gene encoding the PFLA | |
| | | polypeptide (UniProtKB | |
| | | Accession A1A239) | |
| | | Gene encoding the PFLB | |
| | | polypeptide (UniProtKB | |
| | | Accession A1A240) | |
| | | Gene encoding the | |
| | | ADHE polypeptide | |
| | | (UniProtKB Accession | |
| | | A1A067) | |
| | | Gene encoding | |
| | | Saccharomyces | |
| | | cerevisiae STL1 | |
| | | (GeneBank Accession | |
| | | NP_010825 | |
| M11589 | None | Gene encoding | Δgpd2 |
| | | Saccharomycopsis | Δfdh1 |
| | | fibuligera glu0111 | Δfdh2 |
| | | (GeneBank Accession | Δfcy1 |
| | | CAC83969.1) | |
| | | Gene encoding the PFLA | |
| | | polypeptide (UniProtKB | |
| | | Accession A1A239) | |
| | | Gene encoding the PFLB | |
| | | polypeptide (UniProtKB | |
| | | Accession A1A240) | |
| | | Gene encoding the | |
| | | ADHE polypeptide | |
| | | (UniProtKB Accession | |
| | | A1A067) | |
| | | Gene encoding | |
| | | Saccharomyces | |
| | | cerevisiae STL1 | |
| | | (GeneBank Accession | |
| | | NP_010825) | |
| M8841 | None | Gene encoding | Δgpd2 |
| | | Saccharomycopsis | Δfdh1 |
| | | fibuligera glu0111 | Δfdh2 |
| | | (GeneBank Accession | Δfcy1 |
| | | CAC83969.1) | |
| | | Gene encoding the PFLA | |
| | | polypeptide (UniProtKB | |
| | | Accession A1A239) | |
| | | Gene encoding the PFLB | |
| | | polypeptide (UniProtKB | |
| | | Accession A1A240) | |
| | | Gene encoding the | |
| | | ADHE polypeptide | |
| | | (UniProtKB Accession | |
| | | A1A067) | |
| M12962 (wild-type | None | None | None |
| distilling strain) | | | |
| M14028 | Δfcy1 | Gene encoding | Δfcy1 |
| | | Saccharomycopsis | |
| | | fibuligera PEP1 | |
| | | (UniProtKB Accession | |
| | | P22929) (MP818) | |

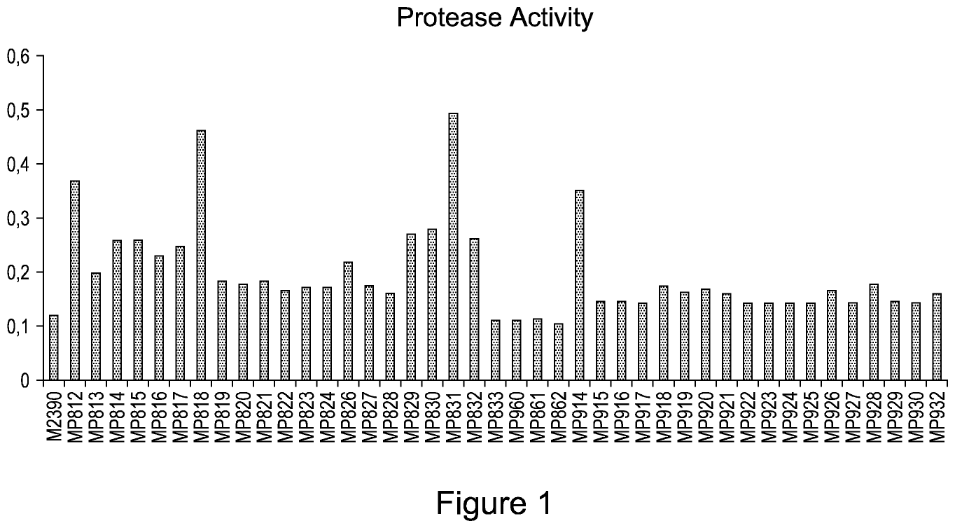

Heterologous protease candidates (summarized in Table 2 above), including three native S. cerevisiae proteases (PEP4, YPS1, YPS3), were expressed in an industrial yeast background. The nucleic acid encoding each of these proteins were codon optimized and then integrated onto the chromosome under control of the yeast constitutive promoter, tef2p (e.g., promoter of the gene encoding the TEF2 polypeptide). These enzymes utilize native signal sequences if from fungal origin or the S. cerevisiae invertase if from bacterial origin. Each of the recombinant yeast host cell was assayed for secreted protease activity using azoalbumin as a substrate. Briefly, cells were grown at 35° C. for 72 hours (h), centrifuged and cell supernatant was added in a 1:1 ratio with a 1% azoalbumin solution and incubated at 35° C. for 4 h. Undigested protein was precipitated with TCA and incubated on ice for 30 minutes (min). The mixture was then filtered and absorbance of filtrate read at 410 nm. The results of the normalized protease activity are presented in FIG. 1. MP812 (C. albicans SAP1), MP814 (Cl. lusitaniae saccharopepsin), MP818 (A. fumigatus PEP1), MP831 (S. fibuligera PEP1), and MP914 (C. dubliniensis SAP1) were found to increased activity. A few other proteases had a moderate of activity (MP813, MP815. MP816, MP817, MP826). Several had little to no activity compared to the wild-type strain.

Next, a subset of these yeast-made proteases were tested in conventional corn mash fermentation in combination with glucoamylase and urea. Strains were inoculated into 20% total solid (TS) corn supplemented with 100% or 50% of a purified glucoamylase enzyme (100%=0.48 amyloglucosidase unit (AGU)/gram of total solids (gTs); 50%=0.24 AGU/gTs) and either 650 ppm or 325 ppm urea. Ethanol and glycerol productions were measured at different points in time with HPLC. Table 4 below compares ethanol and glycerol production over time in MP2390 (wild-type), M11589, M10874 (expressing MP812 in MP2390 background), M12184 (expressing MP812 in M11589 background), M10885 (expressing MP818 in M2390 background) or M12106 (MP818 in M11589 background) strains. As shown in Table 4, strains expressing protease demonstrate improved kinetics, reduced glycerol production and/or urea displacement over parental control.

TABLE 4
Ethanol and glycerol yield of corn fermentation with M2390, M10874, M10885,
M11589, M12184 and M12106 strain in the presence of 100% or 50% GA and 650 or 325 ppm
of urea. Results are provided as g of ethanol or glycerol/L.

| | | | | | | | YP |
|---|---|---|---|---|---|---|---|
| | | | Ethanol | | | | Glycerol |
| GA | | Urea | 22 h | 48 h | 71 h | Potential | 71 h |
| 100% | M2390 | 650 ppm | 72.4 ± 0.629 | 80.0 ± 0.375 | 80.6 ± 0.113 | 80.6 ± 0.113 | 6.3 ± 0.035 |
| | | 325 ppm | 53.3 ± 0.559 | 76.0 ± 0.198 | 79.7 ± 0.926 | 79.7 ± 0.926 | 5.0 ± 0.410 |
| | M10874 | 650 ppm | 75.0 ± 0.049 | 80.4 ± 0.078 | 80.7 ± 0.537 | 80.7 ± 0.537 | 5.9 ± 0.007 |
| | | 325 ppm | 63.2 ± 0.057 | 79.1 ± 0.240 | 80.8 ± 0.113 | 80.8 ± 0.113 | 4.9 ± 0.035 |
| | M10885 | 650 ppm | 77.4 ± 0.071 | 81.6 ± 0.057 | 81.5 ± 0.071 | 81.5 ± 0.071 | 4.9 ± 0.000 |
| | | 325 ppm | 72.2 ± 0.078 | 81.7 ± 0.021 | 82.4 ± 0.269 | 82.4 ± 0.269 | 4.0 ± 0.148 |
| 50% | M11589 | 650 ppm | 80.3 ± 0.332 | 83.7 ± 0.120 | 83.5 ± 0.205 | 83.5 ± 0.205 | 3.2 ± 0.021 |
| | | 325 ppm | 60.7 ± 0.771 | 83.2 ± 0.445 | 83.1 ± 0.820 | 83.1 ± 0.820 | 2.5 ± .0269 |
| | M12184 | 650 ppm | 83.3 ± 2.008 | 84.6 ± 0.007 | 84.7 ± 0.297 | 84.7 ± 0.297 | 3.0 ± 0.007 |
| | | 325 ppm | 70.3 ± 0.057 | 83.8 ± 0.092 | 83.8 ± 0.071 | 83.8 ± 0.071 | 3.1 ± 0.021 |
| | M12106 | 650 ppm | 73.4 ± 0.219 | 76.6 ± 0.276 | 76.5 ± 0.304 | 82.4 ± 0.219 | 3.3 ± 0.028 |
| | | 325 ppm | 73.4 ± 0.262 | 77.4 ± 0.516 | 77.0 ± 0.057 | 82.8 ± 0.499 | 3.1 ± 0.163 |

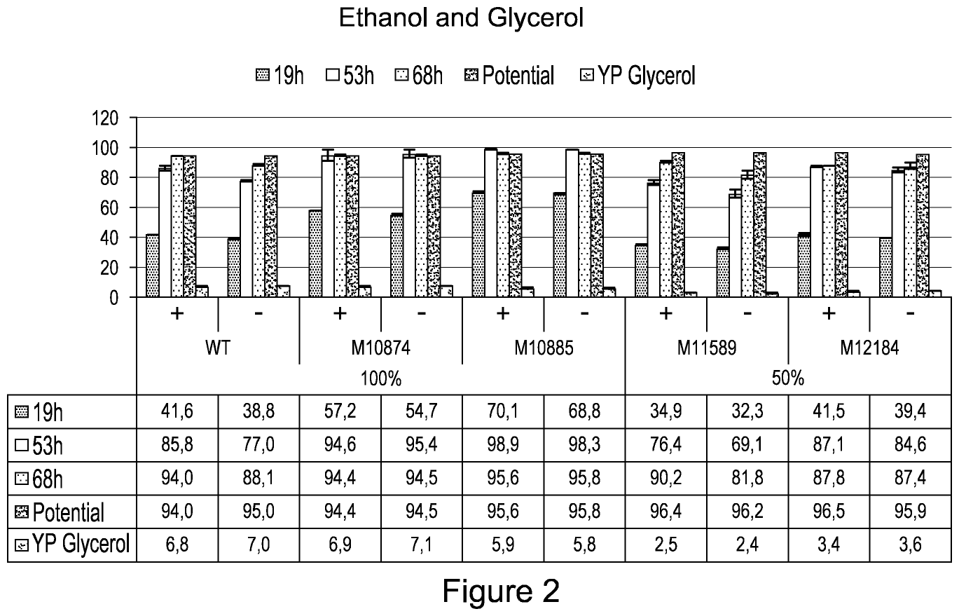

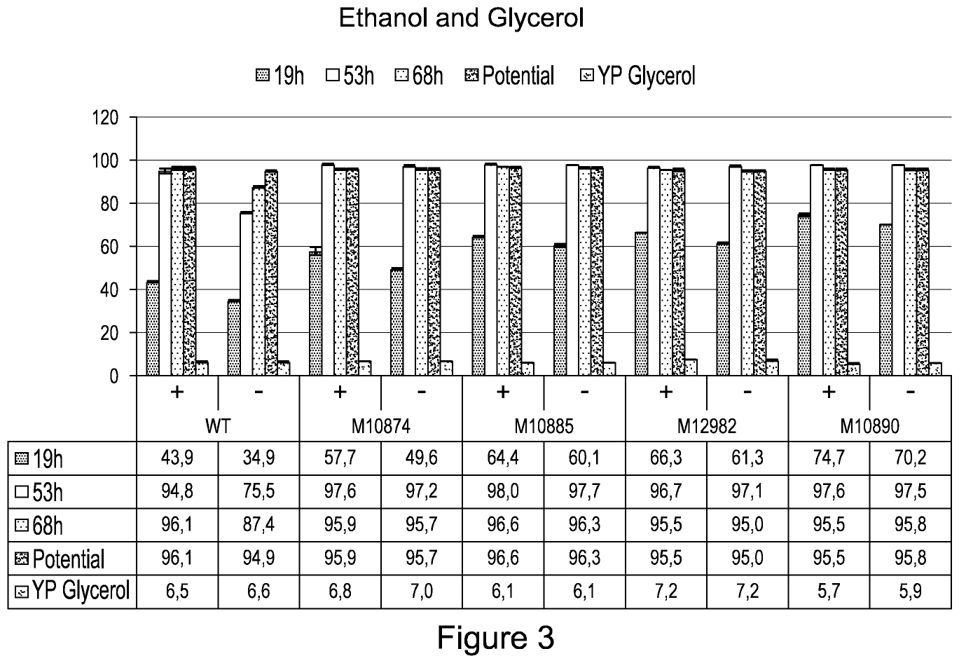

Strains M2390 (wild-type), M10874 (MP814 expressed in a M2390 background), M10885 (MP818 expressed in a M2390 background), M11589, M12184 (MP812 expressed in a M11589 background), M12982 (MP914 expressed in a M2390 background) and M10890 (MP831 expressed in a M2390 background) strains were inoculated into a 23% Ts corn mash fermentation (in the absence of urea supplementation) and in the presence or absence a commercial protease (AYF 117™ in purified form). Protease-expressing strains in a M2390 background were dosed at 100% glucoamylase (0.48 AGU/gTs) whereas protease-expressing strains in a M11589 background were dosed at 50% glucoamylase (0.24 AGU/gTs). Ethanol and glycerol productions were measured at different points in time with HPLC. Results of this fermentation are shown in FIGS. 2 and 3 indicate that, when an heterologous protease is expressed, there is no advantage of supplementing the fermentation medium with a purified protease to increase ethanol yield or reduce glycerol production.

Strain M12962 and M12028 were submitted to a 1.072 OG malted barley fermentation. Briefly, dry malted barley was mashed to create wort with a specific gravity of 1.072. The recombinant strains were tested in shake flasks in this substrate and metabolites were measured by HPLC. As shown in Table 5 below, the M14028 strain has improved kinetics, reduced glycerol (e.g., 14% reduction) and increase in ethanol content (e.g., increase of 1.5%) after 52 h of fermentation.

TABLE 5
Metabolic profile of wild-type distilling strain (M12962) and M12028 strain (MP818
expressed in M12962 background) during malted barley fermentation.

| | | | | | | | Total |
|---|---|---|---|---|---|---|---|
| Strain | Glc | Glycerol | Ethanol | DP4 | DP3 | DP2 | Sugars |
| 24 h | | | | | | | |
| M12962 | 0.29 ± 0.01 | 3.54 ± 0.01 | 69.11 ± 0.52 | 0 ± 0.00 | 6.99 ± 0.00 | 7.43 ± 0.08 | 14.71 ± 0.06 |
| M14028 | 0.35 ± 0.03 | 3.08 ± 0.02 | 73.13 ± 0.07 | 0 ± 0.00 | 6.79 ± 0.01 | 2.20 ± 0.02 | 9.33 ± 0.00 |
| 52 h | | | | | | | |
| M12962 | 0.26 ± 0.02 | 3.56 ± 0.01 | 74.745 ± 0.26 | 0 ± 0.00 | 5.26 ± 0.02 | 0 ± 0.00 | 5.51 ± 0.00 |
| M14028 | 0.40 ± 0.07 | 3.03 ± 0.01 | 75.84 ± 0.33 | 0 ± 0.00 | 4.95 ± 0.24 | 0 ± 0.00 | 5.35 ± 0.00 |

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Guo Z P, Qiu C Y, Zhang L, Ding Z Y, Wang Z X, Shi G Y. Expression of aspartic protease from Neurospora crassa in industrial ethanol-producing yeast and its application in ethanol production. Enzyme Microb Technol. 2011 Feb. 8; 48(2):148-54.
Johnston D B, McAloon A J. Protease increases fermentation rate and ethanol yield in dry-grind ethanol production. Bioresour Technol. 2014 February; 154:18-25.
PCT/U S2012/032443
PCT/US2011/039192
WO 2012/138942

1. A first recombinant yeast host cell comprising a first genetic modification allowing the expression of a heterologous protease, wherein the heterologous protease is:
a) a polypeptide having the amino acid sequence of SEQ ID NO: 2, 6, 8, 10, 12, 14, 30, 36, 38, 40, 42, 52 or 92;
b) a variant having at least 70% identity to the polypeptide of a) and exhibiting proteolytic activity; or
c) a fragment having at least 70% identity to the polypeptide of a) or the variant of b) and exhibiting proteolytic activity.

2.-8. (canceled)

9. The first recombinant yeast host cell of claim 1, having a second genetic modification allowing the expression of a heterologous glucoamylase.

10. The first recombinant yeast host cell of claim 9, wherein the heterologous glucoamylase has the amino acid sequence of SEQ ID NO: 91, is a variant of the amino acid sequence of SEQ ID NO: 91, is a fragment of the amino acid sequence of SEQ ID NO: 91, or is a fragment of the variant.

11. The first recombinant yeast host cell of claim 1 having a third genetic modification for reducing the production of one or more native enzymes that function to produce glycerol or regulate glycerol synthesis.

12. The first recombinant yeast host cell of claim 11, wherein the third genetic modification is for reducing the production of one or more native enzymes that function to produce glycerol.

13. The first recombinant yeast host cell of claim 12, wherein the third genetic modification is for reducing or inhibiting in the expression of the gene encoding the GPD2 polypeptide.

14. The first recombinant yeast host cell of claim 1 having a fourth genetic modification for reducing the production of one or more native enzymes that function to catabolize formate.

15. The first recombinant yeast host cell of claim 14, wherein the fourth genetic modification is for reducing or inhibiting the expression of the genes encoding the FDH1 polypeptide and the FDH2 polypeptide.

16. The first recombinant yeast host cell of claim 1, wherein the first recombinant yeast host cell is from the genus Saccharomyces.

17. The first recombinant yeast host cell of claim 16, wherein the first recombinant yeast host cell is from the species Saccharomyces cerevisiae.

18.-26. (canceled)

27. A process for promoting ethanolic fermentation, the process comprising fermenting a medium with the first recombinant yeast host cell defined in claim 1.

28. The process of claim 27, wherein the medium comprises raw starch.

29. The process of claim 27, wherein the medium is derived from corn.

30. The process of claim 27, wherein the medium is derived from barley.

31. The process of claim 30, wherein the barley is malted barley.

32. A method of producing a heterologous protease in a first recombinant yeast host cell, the method comprising culturing a first recombinant yeast host cell as defined in claim 1 under conditions allowing the expression of the heterologous protease.

33.-44. (canceled)


SEQUENCE LISTING (92 sequences)

SEQ ID NO: 1 - DNA, 1176 bp, Candida albicans
  source 1..1176: mol_type = genomic DNA
atgtttttgaagaacatcttcattgccttggctatcgctttgttggttgatgcttctccagctaaaagatctccaggttttgttaccttggatttcgatgttattaagaccccagttaacgctactggtcaagaaggtaaagttaagagacaagctttgccagttaccttgaacaacgaacatgtttcttacgctgccgatattaccattggttctaacaagcaaaagttcaacgttatcgttgacaccggttcttcagatttgtgggttccagatgcttctgttacttgtgataagccaagaccaggtcaatctgctgatttctgtaaaggtaaaggtatctacaccccaaaatcttctactacctctcaaaatttgggtactccattctacattggttacggtgatggttcttcatctcaaggtacattatacaaggataccgttggttttggtggtgcttctattactaagcaagttttcgctgatatcaccaagacctctattccacaaggtattttgggtattggttacaagactaatgaagctgctggtgattacgataatgttccagtcactttgaagaatcaaggtgttattgctaagaacgcctactccttgtatttgaattctccaaatgctgctaccggtcaaattatcttcggtggtgttgataaggctaagtactctggttctttgattgctgttccagttacatccgatagagaattgagaatcaccttgaactcattgaaggctgttggtaagaacatcaacggtaacattgatgtcttgttggattccggtactaccattacttacttgcaacaagatgttgcccaagatattatcgatgctttccaagctgaattgaagtctgatggtcaaggtcatactttctacgttactgattgtcaaacctccggtactgttgatttcaactttgataacaacgtcaagatctccgttccagcttctgaattcactgctccattgtcttatgctaatggtcaaccatatccaaagtgccaattgttgttgggtatttccgatgctaacatcttgggtgacaatttcttgagatctgcctacttggtttacgatttggatgatgacaagatctctttggcccaagttaagtacacttccgcttctaatattgctgctttgacctaa

SEQ ID NO: 2 - protein, 391 aa, Candida albicans
MFLKNIFIALAIALLVDASPAKRSPGFVTLDFDVIKTPVNATGQEGKVKRQALPVTLNNEHVSYAADITIGSNKQKFNVIVDTGSSDLWVPDASVTCDKPRPGQSADFCKGKGIYTPKSSTTSQNLGTPFYIGYGDGSSSQGTLYKDTVGFGGASITKQVFADITKTSIPQGILGIGYKTNEAAGDYDNVPVTLKNQGVIAKNAYSLYLNSPNAATGQIIFGGVDKAKYSGSLIAVPVTSDRELRITLNSLKAVGKNINGNIDVLLDSGTTITYLQQDVAQDIIDAFQAELKSDGQGHTFYVTDCQTSGTVDFNFDNNVKISVPASEFTAPLSYANGQPYPKCQLLLGISDANILGDNFLRSAYLVYDLDDDKISLAQVKYTSASNIAALT

SEQ ID NO: 3 - DNA, 1197 bp, Aspergillus fumigatus
  source 1..1197: mol_type = genomic DNA
atgaagtccacttctttgttgaccgcttctgttttgttgggttctgcttctgctgctgttcataagttgaaattgaacaaggttccattggacgaacaattatacacccataacattgatgctcacgttagagctttgggtcaaaagtatatgggtatcagaccaaacgtccaccaagaattattggaagaaaactccttgaacgacatgtccagacatgatgttttggttgacaatttcttgaacgcccaatacttctccgaaatctctttgggtactccaccacaaaagttcaaggttgttttggatactggttcctctaatttgtgggttccaggttctgactgttcttctattgcttgtttcttgcacaacaagtacgattcctctgcttcttctacttacaaggctaatggtactgaattcgccattaagtatggttccggtgaattgtctggtttcgtttctcaagataccttgcaaatcggtgatttgaaggttgttaagcaagatttcgctgaagctactaatgaaccaggtttggcttttgcttttggtagattcgatggtattttgggtttgggttacgataccatctccgttaacaaaatcgttccaccattctacaatatgttggatcaaggtttgttggatgaaccagtttttgccttttacttgggtgacactaacaaagaaggtgataactctgaagcttcttttggtggtgttgataagaaccattacactggtgaattgaccaagatcccattgagaagaaaagcttactgggaagttgatttcgatgctattgctttgggtgataacgttgctgaattggaaaacaccggtattatcttggataccggtacttctttgattgctttgccatctactttggccgacttgttgaacaaagaaattggtgctaagaagggtttcaccggtcaatactctattgaatgcgataagagagattccttgccagatttgacttttacattggctggtcataacttcaccattggtccatatgattacaccttggaagtacaaggttcttgcatctcttctttcatgggtatggattttccagaaccagttggtcctttggctattttaggtgatgcttttttgagaaagtggtactccgtttacgatttgggtaacaatgctgttggtttggctaaagctaagtaa

SEQ ID NO: 4 - protein, 398 aa, Aspergillus fumigatus
MKSTSLLTASVLLGSASAAVHKLKLNKVPLDEQLYTHNIDAHVRALGQKYMGIRPNVHQELLEENSLNDMSRHDVLVDNFLNAQYFSEISLGTPPQKFKVVLDTGSSNLWVPGSDCSSIACFLHNKYDSSASSTYKANGTEFAIKYGSGELSGFVSQDTLQIGDLKVVKQDFAEATNEPGLAFAFGRFDGILGLGYDTISVNKIVPPFYNMLDQGLLDEPVFAFYLGDTNKEGDNSEASFGGVDKNHYTGELTKIPLRRKAYWEVDFDAIALGDNVAELENTGIILDTGTSLIALPSTLADLLNKEIGAKKGFTGQYSIECDKRDSLPDLTFTLAGHNFTIGPYDYTLEVQGSCISSFMGMDFPEPVGPLAILGDAFLRKWYSVYDLGNNAVGLAKAK

SEQ ID NO: 5 - DNA, 1221 bp, Clavispora lusitaniae
  source 1..1221: mol_type = genomic DNA
atgcaattgtctgctttggttgctattgctacagctttgattgctggtgctgatgctaaaaagttctctaccaaattgaacaaggtcccaatcgaagaaactttggatgctagatctttttccggttacaccaaatctttggccaacaagtatattggtgcttttggtgctgctggtgttggtgctggttctggtgttcaacaagttgctgaagttccatttgttgccaactctgaacatgaagctccattgactaattacttgaacgctcaatacttcaccgaaatccaattgggtactccaggtcaaactttcaaggttattttggataccggttcctctaatttgtgggttccttctagagactgttcttctttggcttgtttcttgcataccaagtacgatcacgatgaatcttctacttacaaggctaacggttccgaattctctattcaatatggttcaggtgctatggaaggttacatctctcaagatgttttggccattggtgatttggttatcccaaagcaagatttcgctgaagctacttctgaaccaggtttggcttttgctttcggtaagtttgatggtattttgggtttggcctacgataccatttctgttaacaagatagttccaccagtctacaacgctattgctcaaggtttgttggatgctccacaatttggtttttacttgggtgataccaacaagaacgaagaaaatggtggtgttgctacttttggtggttatgatgaagctttgttcaagggtgatttgacttggttgccagttagaagaaaagcttactgggaagtttctttcgacggtattggtttgggtgatgaatatgctgaattgactgctacaggtgctgctattgatactggtacttctttgattaccttgccatcttcattggccgaaattatcaatgctaaaattggtgctaccaagtcctggtctggtcaatatcaagttgattgtgctaccagagataacttgccagatttgacattgacattcgctggttacaacttcactttgtctccatacgattacaccttggaagtttccggttcttgtatttctgctttcactccaatggatttcccagaacctattggtgacttggctatagttggtgatgctttcttgagaagatattactccgtttacgacttgaagaaggatgctgttggtttggctccagctaaataa

SEQ ID NO: 6 - protein, 406 aa, Clavispora lusitaniae
MQLSALVAIATALIAGADAKKFSTKLNKVPIEETLDARSFSGYTKSLANKYIGAFGAAGVGAGSGVQQVAEVPFVANSEHEAPLTNYLNAQYFTEIQLGTPGQTFKVILDTGSSNLWVPSRDCSSLACFLHTKYDHDESSTYKANGSEFSIQYGSGAMEGYISQDVLAIGDLVIPKQDFAEATSEPGLAFAFGKFDGILGLAYDTISVNKIVPPVYNAIAQGLLDAPQFGFYLGDTNKNEENGGVATFGGYDEALFKGDLTWLPVRRKAYWEVSFDGIGLGDEYAELTATGAAIDTGTSLITLPSSLAEIINAKIGATKSWSGQYQVDCATRDNLPDLTLTFAGYNFTLSPYDYTLEVSGSCISAFTPMDFPEPIGDLAIVGDAFLRRYYSVYDLKKDAVGLAPAK

SEQ ID NO: 7 - DNA, 1218 bp, Saccharomyces cerevisiae
  source 1..1218: mol_type = genomic DNA
atgttcagcttgaaagcattattgccattggccttgttgttggtcagcgccaaccaagttgctgcaaaagtccacaaggctaaaatttataaacacgagttgtccgatgagatgaaagaagtcactttcgagcaacatttagctcatttaggccaaaagtacttgactcaatttgagaaagctaaccccgaagttgttttttctagggagcatcctttcttcactgaaggtggtcacgatgttccattgacaaattacttgaacgcacaatattacactgacattactttgggtactccacctcaaaacttcaaggttattttggatactggttcttcaaacctttgggttccaagtaacgaatgtggttcgttggcttgtttcctacattctaaatacgatcatgaagcttcatcaagctacaaagctaatggtactgaatttgccattcaatatggtactggttctttggaaggttacatttctcaagacactttgtccatcggggatttgaccattccaaaacaagacttcgctgaggctaccagcgagccgggcttaacatttgcatttggcaagttcgatggtattttgggtttgggttacgataccatttctgttgataaggtggtccctccattttacaacgccattcaacaagatttgttggacgaaaagaaatttgccttttatttgggagacacttcaaaggatactgaaaatggcggtgaagccacctttggtggtattgacgagtctaagttcaagggcgatatcacttggttacctgttcgtcgtaaggcttactgggaagtcaagtttgaaggtatcggtttaggcgatgagtacgccgaattggagagccatggtgccgccatcgatactggtacttctttgattaccttgccatcaggattagctgaaatgattaatgctgaaattggtgccaagaagggttggaccggtcaatatactctagactgtaacaccagagacaatctacctgatctgattttcaacttcaatggctacaacttcactattgggccatacgattacacgcttgaagtttcaggctcctgtatctctgcaattacaccaatggatttcccagaacctgttggcccactggccatcgttggtgatgccttcttgcgtaaatactattctatttacgatttgggcaacaatgcggttggtttggccaaagcaatttga

SEQ ID NO: 8 - protein, 405 aa, Saccharomyces cerevisiae
MFSLKALLPLALLLVSANQVAAKVHKAKIYKHELSDEMKEVTFEQHLAHLGQKYLTQFEKANPEVVFSREHPFFTEGGHDVPLTNYLNAQYYTDITLGTPPQNFKVILDTGSSNLWVPSNECGSLACFLHSKYDHEASSSYKANGTEFAIQYGTGSLEGYISQDTLSIGDLTIPKQDFAEATSEPGLTFAFGKFDGILGLGYDTISVDKVVPPFYNAIQQDLLDEKRFAFYLGDTSKDTENGGEATFGGIDESKFKGDITWLPVRRKAYWEVKFEGIGLGDEYAELESHGAAIDTGTSLITLPSGLAEMINAEIGAKKGWTGQYTLDCNTRDNLPDLIFNFNGYNFTIGPYDYTLEVSGSCISAITPMDFPEPVGPLAIVGDAFLRKYYSIYDLGNNAVGLAKAI

SEQ ID NO: 9 - DNA, 1191 bp, Yarrowia lipolytica
  source 1..1191: mol_type = genomic DNA
atgaagttcactgctgctgtttctgttttggctgctgcaggttcagtttctgcagctgtttcaaaagtttccatcaacaagatgtccaccgctgaattattgggtaaagaaaacggtttcgaagatcacttgagaatgatgggtcaaaagtacatgggtaaattccaaaagttgggtgaattcaacgaattggcctccattcaagatgtttctaattctccattgaccaactacttgaacgcccaatattacaccgaaatcgaaattggtactccaccacaaaagttcaacgttattttggataccggttcctctaatttgtgggttccatctgttcaatgcaactctattgcttgctacttgcaccaaaagtatgattctgctgcttcctcatcttacaaggctaatggtactgctttcgaaatccaatatggttccggttctatggaaggtttcgtttctcaagatactttgaagttgggttccttggttttgccagaacaagattttgctgaagctacttctgaaccaggtttggcttttgcttttggtaaattcgatggtattttgggtttggcctacgataccatttctgttaacaagatagttccaccagtttacaacgccgttaatagaggtttgttggacaagaatcaattctcctttttcttgggtgataccaacaaaggtactgatggtggtgttgctactttcggtggtgtagatgaagattacttcgaaggtaaaattacctggttgccagttagaagaaaagcctattgggaagtcgaattcaactccattactttgggtgatcaaactgccgaattggttaatactggtgctgctattgataccggtacttctttgttggctttgccatctggtttggctgaagttttgaattctgaaattggtgctactaagggttggtctggtcaatatactgttgaatgcgataaggttgattccttgccagatttgacttttaacttcgctggttacaacttcaccattggtccaagagattacaccttggaattgtctggttcttgtgtttctgctttcaccggttttgatattccagctccagttggtccaattgccattattggtgatgctttcttgagaagatattactccgtttacgatttggatcatgatgctgttggtttggcaaaagctaagtaa

SEQ ID NO: 10 - protein, 396 aa, Yarrowia lipolytica
MKFTAAVSVLAAAGSVSAAVSKVSINKMSTAELLGKENGFEDHLRMMGQKYMGKFQKLGEFNELASIQDVSNSPLTNYLNAQYYTEIEIGTPPQKFNVILDTGSSNLWVPSVQCNSIACYLHQKYDSAASSSYKANGTAFEIQYGSGSMEGFVSQDTLKLGSLVLPEQDFAEATSEPGLAFAFGKFDGILGLAYDTISVNKIVPPVYNAVNRGLLDKNQFSFFLGDTNKGTDGGVATFGGVDEDYFEGKITWLPVRRKAYWEVEFNSITLGDQTAELVNTGAAIDTGTSLLALPSGLAEVLNSEIGATKGWSGQYTVECDKVDSLPDLTFNFAGYNFTIGPRDYTLELSGSCVSAFTGFDIPAPVGPIAIIGDAFLRRYYSVYDLDHDAVGLAKAK

SEQ ID NO: 11 - DNA, 1227 bp, Meyerozyma guilliermondii
  source 1..1227: mol_type = genomic DNA
atgaagttgtccatctctgttttgggtgctgttgcttttgctttgtttggttgtgctgatgctgctgttcattctgctaaattgaacaagatcccagtcgaagaaactttggctgctcatagattcaaagaatacacttctggtttggccgctaaatacttgactgctttttctacctctgaaggtattaccgatcaaacccaacaacaaatcttgcaacaagttccattcgtcgatggtaaatacgattccgatttgtccaattacgttaacgctcaatacttcaccgaaatccaattgggtactccaggtcaaactttcaaggttattttggataccggttcctctaatttgtgggttccttctgctgactgttcttctttggcttgtttcttgcataccaagtacgatcacgattcttcatctacttacaaggctaacggttccgaattctctattcaatatggttctggtgctatggaaggttacgtttctagagatactttggctttgggtgatttgatcatcccaagacaagattttgctgaagctacttctgaaccaggtttggcatttgcttttggtaaattcgatggtattttgggtttggcctacaacaccatttctgttaacaagatagttccaccaatctacaacgccattgatcaaggtttgttggatgaaccagttttcgcttttagattgggtgatacttctaaggacgaaaacgatggtggtgttgctacttttggtggttatgataagtctcaattcaccggtaagattacctggttgccagttagaagaaaagcttactgggaagtttctttcgaaggtatcggtttaggtgatgaatacgctgaattgacttctactggtgctgctattgatactggtacttctttgattaccttgccatcttccttggccgaaattatgaataccaaaattggtgctaccaagtcctggtctggtcaatatcaaattgattgcgaaaagagagactccttgccagatttgactttgaatttctctggttacaacttcaccttgtccccatatgattacactttggaagttggtggttcctgcatttcagtttttactccaatggatttcccagaacctatcggtgatttggctatagttggtgatgctttcttgagaagatattactccatctacgacttgaagaaggatgctgttggtttggctaaatccgtttaa

SEQ ID NO: 12 - protein, 408 aa, Meyerozyma guilliermondii
MKLSISVLGAVAFALFGCADAAVHSAKLNKIPVEETLAAHRFKEYTSGLAAKYLTAFSTSEGITDQTQQQILQQVPFVDGKYDSDLSNYVNAQYFTEIQLGTPGQTFKVILDTGSSNLWVPSADCSSLACFLHTKYDHDSSSTYKANGSEFSIQYGSGAMEGYVSRDTLALGDLIIPRQDFAEATSEPGLAFAFGKFDGILGLAYNTISVNKIVPPIYNAIDQGLLDEPVFAFRLGDTSKDENDGGVATFGGYDKSQFTGKITWLPVRRKAYWEVSFEGIGLGDEYAELTSTGAAIDTGTSLITLPSSLAEIMNTKIGATKSWSGQYQIDCEKRDSLPDLTLNFSGYNFTLSPYDYTLEVGGSCISVFTPMDFPEPIGDLAIVGDAFLRRYYSIYDLKKDAVGLAKSV

SEQ ID NO: 13 - DNA, 1188 bp, Aspergillus fumigatus
  source 1..1188: mol_type = genomic DNA
atggtcgttttctctaaggttaccgctgttgttgttggtttgtctactatagtttctgccgttccagttgttcaacctagaaaaggtttcaccatcaatcaagttgctagaccagttaccaacaaaaagactgttaatttgccagctgtttacgctaacgctttgactaagtatggtggtactgttccagattctgttaaggctgctgcttcttctggttctgctgttactactccagaacaatacgattctgaatacttgaccccagttaaggttggtggtacaactttgaatttggatttcgatactggttccgctgatttgtgggttttttcttctgaattgtccgcctctcaatcttctggtcatgctatctacaaaccatctgctaatgctcaaaagttgaacggttacacctggaagattcaatatggtgatggttcttctgcttccggtgatgtttacaaagatactgttactgttggtggtgttaccgctcaatctcaagctgttgaagctgcttctcatatctcttcacaattcgttcaagataaggacaacgacggtttgttgggtttggctttttcatctattaacaccgtttctccaagacctcaaactactttctttgataccgtcaagtcccaattggattctcctttgtttgctgttaccttgaaatatcatgctccaggtacttacgatttcggttacattgataactccaagttccaaggtgaattgacctacactgatgttgattcttctcaaggtttctggatgtttactgctgatggttatggtgtaggtaatggtgctccaaactctaactctatttctggtattgctgataccggtactaccttgttgttattggatgattctgttgttgccgactactacagacaagtttctggtgctaagaactccaatcaatacggtggttatgttttcccatgttctactaagttgccatctttcactactgttatcggtggttacaatgctgttgttcctggtgaatatatcaactacgctccagttactgatggttcctctacttgctacggtggtattcaatctaattctggtttgggtttctccatcttcggtgacatttttttgaagtctcaatacgtcgttttcgactctcaaggtccaagattgggttttgctccacaagcttaa

SEQ ID NO: 14 - protein, 395 aa, Aspergillus fumigatus
MVVFSKVTAVVVGLSTIVSAVPVVQPRKGFTINQVARPVTNKKTVNLPAVYANALTKYGGTVPDSVKAAASSGSAVTTPEQYDSEYLTPVKVGGTTLNLDFDTGSADLWVFSSELSASQSSGHAIYKPSANAQKLNGYTWKIQYGDGSSASGDVYKDTVTVGGVTAQSQAVEAASHISSQFVQDKDNDGLLGLAFSSINTVSPRPQTTFFDTVKSQLDSPLFAVTLKYHAPGTYDFGYIDNSKFQGELTYTDVDSSQGFWMFTADGYGVGNGAPNSNSISGIADTGTTLLLLDDSVVADYYRQVSGAKNSNQYGGYVFPCSTKLPSFTTVIGGYNAVVPGEYINYAPVTDGSSTCYGGIQSNSGLGFSIFGDIFLKSQYVVFDSQGPRLGFAPQA

SEQ ID NO: 15 - DNA, 1710 bp, Saccharomyces cerevisiae
  source 1..1710: mol_type = genomic DNA
atgaaactgaaaactgtaagatctgcggtcctttcgtcactctttgcatcgcaggttctcggtaagataataccagcagcaaacaagcgcgacgacgactcgaattccaagttcgtcaagttgccctttcataagctttacggggattcgctagaaaatgtgggaagcgacaaaaaaccggaagtacgcctattgaagagggctgacggttatgaagaaattataattaccaaccagcaaagtttctattcggtggacttggaagtgggcacgccaccacagaacgtaacggtcctggtggacacaggctcctctgatctatggattatgggctcggataatccatactgttcttcgaacagtatgggtagtagccggagacgtgttattgacaaacgtgatgattcgtcaagcggcggatctttgattaatgatataaacccatttggctggttgacgggaacgggcagtgccattggccccactgctacgggcttaggaggcggttcaggtacggcaactcaatccgtgcctgcttcggaagccaccatggactgtcaacaatacgggacattttccacttccggctcttctacatttagatcaaacaacacctatttcagtattagctacggtgatgggacttttgcctccggtacttttggtacggatgttttggatttaagcgacttgaacgttaccgggttgtcttttgccgttgccaatgaaacgaattctactatgggtgtgttaggtattggtttgcccgaattagaagtcacctattctggctctactgcgtctcatagtggaaaagcttataaatacgacaacttccccattgtattgaaaaattctggtgctatcaaaagcaacacatattctttgtatttgaacgactcggacgctatgcatggcaccattttgttcggagccgtggaccacagtaaatataccggcaccttatacacaatccccatcgtaaacactctgagtgctagtggatttagctctcccattcaatttgatgtcactattaatggtatcggtattagtgattctgggagtagtaacaagaccctgactaccactaaaatacctgctttgttggattccggtactactttgacttatttacctcaaacagtggtaagtatgatcgctactgaactaggtgcgcaatactcttccaggatagggtattacgtattggactgtccatctgatgatagtatggaaatagtgttcgattttggtggttttcacatcaatgcaccactttcgagttttatcttgagtactggcactacatgtcttttaggtattatcccaacgagtgatgacacaggtaccattttgggtgattcatttttgactaacgcgtacgtggtttatgatttggagaatcttgaaatatccatggcacaagctcgctataataccacaagcgaaaatatcgaaattattacatcctctgttccaagcgccgtaaaggcaccaggctatacaaacacttggtctacaagtgcatctattgttaccggtggtaacatatttactgtaaattcctcacaaactgcttcctttagcggtaacctgacgaccagtactgcatccgccacttctacatcaagtaaaagaaatgttggtgatcatatagttccatctttacccctcacattaatttctcttctttttgcattcatctga

SEQ ID NO: 16 - protein, 569 aa, Saccharomyces cerevisiae
MKLKTVRSAVLSSLFASQVLGKIIPAANKRDDDSNSKFVKLPFHKLYGDSLENVGSDKKPEVRLLKRADGYEEIIITNQQSFYSVDLEVGTPPQNVTVLVDTGSSDLWIMGSDNPYCSSNSMGSSRRRVIDKRDDSSSGGSLINDINPFGWLTGTGSAIGPTATGLGGGSGTATQSVPASEATMDCQQYGTFSTSGSSTFRSNNTYFSISYGDGTFASGTFGTDVLDLSDLNVTGLSFAVANETNSTMGVLGIGLPELEVTYSGSTASHSGKAYKYDNFPIVLKNSGAIKSNTYSLYLNDSDAMHGTILFGAVDHSKYTGTLYTIPIVNTLSASGFSSPIQFDVTINGIGISDSGSSNKTLTTTKIPALLDSGTTLTYLPQTVVSMIATELGAQYSSRIGYYVLDCPSDDSMEIVFDFGGFHINAPLSSFILSTGTTCLLGIIPTSDDTGTILGDSFLTNAYVVYDLENLEISMAQARYNTTSENIEIITSSVPSAVKAPGYTNTWSTSASIVTGGNIFTVNSSQTASFSGNLTTSTASATSTSSKRNVGDHIVPSLPLTLISLLFAFI

SEQ ID NO: 17 - DNA, 1374 bp, Yarrowia lipolytica
  source 1..1374: mol_type = genomic DNA
atgaagttctccttggttaccttgactactttgtgtgcttctgctttggctgctccaactgctaaaaaatccttgaagatcgacttccaaaagcaattggctgattctggtactacttcccaagatccaaatcaattgggtggtgctcaaggtaaagttccacatgaagttgaattgaccaaccacgttgtttactacttggctgaagttgctttgggtactccaccacaaaagttccaaattgatattgacaccggttcctctgatttgtgggttaaggctgatggttctccaggtgcttattctagaaattcttcttctacctggtcccattacgctgacaatttttacattgcttacggtgatcaaacctccgcttctggtgattgggctactgaaactttgggttttgctgatgctcaaatccctaagttcatttttggtgaagctacttctgctacctctcaaccagtttttggtattggttactctggtattgaagcctccattcatcaacctaatgctttcacttacgacaacttcccaattagattggccaaagaaggtttcgttaataccccagcttactccttgtacttgaatgattttgctgctaagaccggttctgttttgtttggtgctgttgataagtccaagatcgatggttctttgactattttgccaaccatcaaggatcaaactaccgactctaagccaaaagaatttttggtcaccttgaactccatcgacatcaatgttaatggtactaccacaaacgctttggataagaccagacatgttttgttggattccggtacttctttgacatacttgccacctcaaactactagaaccattgcccaaaagtttcaattattgcaagtttcaggtggttggggtttgaccaagaaacaagttgatgctttgccagatactgctaccttggattataacttgcaaggtgctcatgttggtgttaaggttaaggatttgttcaccttgggtaagaactacttgaatcaacaattgtacatcgaatacaacggtgtcagagaaccattctaccaaattttgattgctgatggtggtgatagaggtccaggtaatgatgaaccagttgaattagccaagttcatcttcggtgattcattcttgagatccgcctatgttgtttacgatattggtgctgataagatcgctgtttctcaagctaaatttggttctggttctgctgaagatttggccgatattcaaattgaagataagggtggtattccagctgctactgctgcttctgatccagtttggactcaaaatgctccaattgaaacctccgttaactacaaccctcaaatctacagattgtccacctaa

SEQ ID NO: 18 - protein, 457 aa, Yarrowia lipolytica
MKFSLVTLTTLCASALAAPTAKKSLKIDFQKQLADSGTTSQDPNQLGGAQGKVPHEVELTNHVVYYLAEVALGTPPQKFQIDIDTGSSDLWVKADGSPGAYSRNSSSTWSHYADNFYIAYGDQTSASGDWATETLGFADAQIPKFIFGEATSATSQPVFGIGYSGIEASIHQPNAFTYDNFPIRLAKEGFVNTPAYSLYLNDFAAKTGSVLFGAVDKSKIDGSLTILPTIKDQTTDSKPKEFLVTLNSIDINVNGTTTNALDKTRHVLLDSGTSLTYLPPQTTRTIAQKFQLLQVSGGWGLTKKQVDALPDTATLDYNLQGAHVGVKVKDLFTLGKNYLNQQLYIEYNGVREPFYQILIADGGDRGPGNDEPVELAKFIFGDSFLRSAYVVYDIGADKIAVSQAKFGSGSAEDLADIQIEDKGGIPAATAASDPVWTQNAPIETSVNYNPQIYRLST

SEQ ID NO: 19 - DNA, 1650 bp, Meyerozyma guilliermondii
  source 1..1650: mol_type = genomic DNA
atgagacctttcttgtctgttgcttctttggcttatgctgctgtttgtgctgtttctgcagcagctgtttcagatactgaaaatgctggtaataccccattcaagatcgacttcgaaattcacagaggttcttctacttgggatatggctagacataagagaggtgaattggttaagagagatggttccttggatatggaaatcaagaacgaaaacaccttttacttggccgaattgaagttcggttctaacgaaaacaaagttggtgttttggttgacaccggttcttcagatttgtggattatgtctcatgacttgagatgcgaagctgtttctcaatctgctaagagaaagagagaaagattgatcaccttgccagaagacgaaactaagaaaggttcttcaaaaggtgcccacaaagaagttggtcacgaaaaagctggtttctacactactattgaattgaccgaaggtggtgattatgctactgcttcttatgctcacgaaacaaacacttgtactggtcatggttctttcgctactgctaattctgattccttcaagagaaattcttctgctccagccttctctatttcttacgctgatggtactgatgctaatggtgtttggggtcatgatgatgttattattggtaacaccaccgtcaagtctttgtcttttgctgttgctaacgaaacctcttctaatgttggtgtattgggtattggtttgatcggtttggaagttacctcttcttttactggttcttctggtcaatctggtggttacacttattctaacttgccattgaagttgaaggacgatggtatcatccacaagaacgttttctcattatacttgggtgaagaatccgactcctctggttctattttgtttggtgcagttgattccgctaagtactctggtactttacaaactgttccaatggtcaactcctactctcaatatactgataccccaatcagaatcgaagttgctttgaacgctatgcaaatcgaatccggttctaagaactacaccatttcttctaaagctcacgccgttgttttggatacaggttctacttactcctacattttcgaagatatgttggaaaacgttgccaacactgttggtgctagatattcttcttcagctggtgcttatgttatgtcctgcattgatgatgataacgccaagattaccttggacttttctggtaacaagttggaaattccattgtccgctattcaagctccagcttcttcaaatggtaacacttgtttcttgaccttgttgccacaatcctctgattctagatatgttttgttcggtgacaacatcttgagacacatgtacatcgtttacgatttggatgactacgaagtttccttcgctcaagttaagttcactaacgacgaaaacgtcgaagttgtcacttcttctattccatctgcttctaaggctccaaactactcttctacatctattgcctctaacgaaggtgaatccatcatttctggttctgctaatttgggtactggttcctcttcttcatcatctggtcattctaaatccggtggttccattttatctgtcccattggttatggctttcgttgctattggttctttgttcgttgtttgttaa

SEQ ID NO: 20 - protein, 549 aa, Meyerozyma guilliermondii
MRPFLSVASLAYAAVCAVSAAAVSDTENAGNTPFKIDFEIHRGSSTWDMARHKRGELVKRDGSLDMEIKNENTFYLAELKFGSNENKVGVLVDTGSSDLWIMSHDLRCEAVSQSAKRKRERLITLPEDETKKGSSKGAHKEVGHEKAGFYTTIELTEGGDYATASYAHETNTCTGHGSFATANSDSFKRNSSAPAFSISYADGTDANGVWGHDDVIIGNTTVKSLSFAVANETSSNVGVLGIGLIGLEVTSSFTGSSGQSGGYTYSNLPLKLKDDGIIHKNVFSLYLGEESDSSGSILFGAVDSAKYSGTLQTVPMVNSYSQYTDTPIRIEVALNAMQIESGSKNYTISSKAHAVVLDTGSTYSYIFEDMLENVANTVGARYSSSAGAYVMSCIDDDNAKITLDFSGNKLEIPLSAIQAPASSNGNTCFLTLLPQSSDSRYVLFGDNILRHMYIVYDLDDYEVSFAQVKFTNDENVEVVTSSIPSASKAPNYSSTSIASNEGESIISGSANLGTGSSSSSSGHSKSGGSILSVPLVMAFVAIGSLFVVC

SEQ ID NO: 21 - DNA, 1527 bp, Saccharomyces cerevisiae
  source 1..1527: mol_type = genomic DNA
atgaaacttcaattggcggcagtggctacattagcagtcttaactagtccggcattcggtagagtacttcccgatgggaaatacgtcaagattcccttcacaaaaaaaaagaacggcgacaatggtgaactcagcaagagatcgaacggccatgaaaaatttgtactagctaacgagcaaagcttttattctgttgagctagccattggtacaccttcacaaaacctcactgtgctgttagacacaggctcagccgacttatgggttcctggcaagggaaacccctactgcggttctgtgatggactgtgaccagtacggcgtgttcgacaagaccaagtcgtccacgttcaaagccaacaagtcctcgcctttttatgccgcttacggtgacggaacctatgcagaaggtgcatttggccaagacaaactaaagtacaacgaattagacctcagcggtctatcgtttgccgtggccaacgaatctaactcaacctttggtgtgctcgggatcggcctttccacgcttgaagtcacctattctggaaaagtcgctattatggacaagagaagctacgagtatgataactttcccctgttcctaaaacattctggagccatcgatgcaactgcatactctcttttcctaaatgacgagtcacagtcctccggcagcatcctcttcggcgctgtagatcacagcaagtacgagggccaactgtacactatcccgttggttaatctttataagtcgcagggttatcagcacccggtggcgttcgatgtcactttacagggcttaggactgcaaaccgacaagcgcaacatcacattgaccaccaccaagctcccagcccttctcgattcaggcacaacgctaacatatctgccctcccaggcagtggctttgctagcaaagagcttaaatgcctcgtattccaagacattgggttattatgagtacacgtgtccctcgagcgacaacaaaaccagcgtggccttcgacttcggtggcttccgtatcaacgctcctctatccgactttactatgcagaccagtgtggagacctgtgtcttggcaataattccacaagcgggcaacgccaccgctatccttggtgattccttcttgagaaacgcctacgtggtctacgatttggataactacgagatttccctagctcaagccaagtatggcacggggaaagagaacgtcgaagtcatcaaatctaccgttcccagtgcaataagggcccccagttacaacaacacttggtctaactacgcctccgccacgtccggtggtaatatttttaccaccgtgcgcactttcaatggcaccagtactgccaccactacgaggtcaaccaccaccaagaagacaaactctaccactactgcaaagtcgactcataaaagcaagagggcactccagagggctgctaccaactccgcttccagtatacgctctaccttgggtttactgctagtcccctccttactcatcctttccgttttcttttcgtaa

SEQ ID NO: 22 - protein, 508 aa, Saccharomyces cerevisiae
MKLQLAAVATLAVLTSPAFGRVLPDGKYVKIPFTKKKNGDNGELSKRSNGHEKFVLANEQSFYSVELAIGTPSQNLTVLLDTGSADLWVPGKGNPYCGSVMDCDQYGVFDKTKSSTFKANKSSPFYAAYGDGTYAEGAFGQDKLKYNELDLSGLSFAVANESNSTFGVLGIGLSTLEVTYSGKVAIMDKRSYEYDNFPLFLKHSGAIDATAYSLFLNDESQSSGSILFGAVDHSKYEGQLYTIPLVNLYKSQGYQHPVAFDVTLQGLGLQTDKRNITLTTTKLPALLDSGTTLTYLPSQAVALLAKSLNASYSKTLGYYEYTCPSSDNKTSVAFDFGGFRINAPLSDFTMQTSVGTCVLAIIPQAGNATAILGDSFLRNAYVVYDLDNYEISLAQAKYGTGKENVEVIKSTVPSAIRAPSYNNTWSNYASATSGGNIFTTVRTFNGTSTATTTRSSTTKKTSSTTTAKSTHKSKRALQRAATNSASSIRSTLGLLLVPSLLILSVFFS

SEQ ID NO: 23 - DNA, 1185 bp, Candida tropicalis
  source 1..1185: mol_type = genomic DNA
atggccaccattttcttgttcaccaagaacgtttttattgctttggctttcgctttgttcgctcaaggtttgactattccagatggtattgaaaagagaaccgacaaggttgtttctttggatttcaccgttatcagaaagccattcaatgctactgcccatagattgatccaaaagagatctgacgttccaaccaccttgattaacgaaggtccatcttatgctgctgatatcgttgttggttccaatcaacaaaagcaaaccgttgttattgacaccggttcttcagatttgtgggttgttgatactgatgctgaatgtcaagttacctactctggtcaaactaacaacttctgtaagcaagaaggtactttcgacccatcttcttcatcttcagcccaaaacttgaatcaagacttctccattgaatacggtgacttgacatcttctcaaggttctttctacaaggatactgttggtttcggtggtatctccatcaagaatcaacaattcgctgatgttactaccacctctgttgatcaaggtattatgggtattggtttcactgctgttgaagctggttataacttgtactctaacgttccagttaccttgaagaagcaaggtatcattaacaagaacgcctactcttgcgatttgaactctgaagatgcttctaccggtaagattatctttggtggtgttgataacgctaagtacactggtactttgactgctttgccagttacttcttctgttgaattgagagttcacttgggttccattaacttcgatggtacttctgtttctaccaacgcagatgttgttttggattctggtactaccattacctacttctctcaatctactgctgataagttcgctagaatagttggtgctacttgggattctagaaacgaaatctacagattgccatcctgtgatttgtctggtgatgctgttgttaatttcgatcaaggtgttaagatcaccgtcccattgtctgaattgatcttgaaggattccgattcctccatttgctacttcggtatctcaagaaatgatgccaatatcttgggtgacaactttttgagaagagcctacatcgtttacgatttggatgataagaccatctctttggcccaagttaagtatacctcctcctctgatatttccgctttgtaa

SEQ ID NO: 24 - protein, 394 aa, Candida tropicalis
MATIFLFTKNVFIALAFALFAQGLTIPDGIEKRTDKVVSLDFTVIRKPFNATAHRLIQKRSDVPTTLINEGPSYAADIVVGSNQQKQTVVIDTGSSDLWVVDTDAECQVTYSGQTNNFCKQEGTFDPSSSSSAQNLNQDFSIEYGDLTSSQGSFYKDTVGFGGISIKNQQFADVTTTSVDQGIMGIGFTAVEAGYNLYSNVPVTLKKQGIINKNAYSCDLNSEDASTGKIIFGGVDNAKYTGTLTALPVTSSVELRVHLGSINFDGTSVSTNADVVLDSGTTITYFSQSTADKFARIVGATWDSRNEIYRLPSCDLSGDAVVNFDQGVKITVPLSELILKDSDSSICYFGISRNDANILGDNFLRRAYIVYDLDDKTISLAQVKYTSSSDISAL

SEQ ID NO: 25 - DNA, 1191 bp, Clavispora lusitaniae
  source 1..1191: mol_type = genomic DNA
atgttgcctttgttgaccatcttttctttggctttgactttggttaacggtttggctattccaaagaacgaagcttctttggttggtagatctgatccagctccattgaagttggatttcagagttcaaaagaacgtcggtaacatttccgctagatctttttggaatgaagttcatgccttgagacaccaatctaagaaaagaggttcttacccagaaaccttgaaggatgttgatgatgtttcctacgttgttgatatctacgttggttccgataagcaaaaggctactgttgttttggataccggttcttctgatttgtggatctcttcacaagattctggttctggtagttacgatccatcttcttcatctgattctcaagatactggtaacgccttcgatatttcttatggtgatggtactacttccaacggtgaatattacactgactcattttctttctcttccaccggtaatcctttgttgtcctcatttcaattggcttccacctcctcttctattgatggtgctgctggtattttgggtattggtgataagaatactgaagcctccgaacaacaatacgatgatttgccatgggctttacaaaaagctggtaaaactccaaaggcctcctactcattatacttgggttcaggtgatggtgcatctggttcagttatttttggtggtattgacaccgaaaagtactctggtgaattgactaagtacccagttgatacttctaatggtccaggtttgtttgttaacgttgctaccttttctgttgacggtaaggatttttctactgatgctccattcttgttggactctggtacttctttgggttttgttccacaagatgttcaagactacttggactctattttcaacccaactatggttcaagaaggtgccattacttacagacaagtttcttgtgatcaacctaccgataagttcttcactttggatttcggtcaaaacaagatcaccttctcttacgctgatgccatttctcaacaagacgaaaacacttgtttgttgggtttcacctatcacgatgatacctacattttgggtgacgtttttttgagaaacgcctacgtttactacgatttgaccgatggttctatctctttggctcaagctaagtacacttctgcttctaatatcgtttccgcctaa

SEQ ID NO: 26 - protein, 396 aa, Clavispora lusitaniae
MLPLLTIFSLALTLVNGLAIPKNEASLVGRSDPAPLKLDFRVQKNVGNISARSFWNEVHALRHQSKKRGSYPETLKDVDDVSYVVDIYVGSDKQKATVVLDTGSSDLWISSQDSGSGSYDPSSSSDSQDTGNAFDISYGDGTTSNGEYYTDSFSFSSTGNPLLSSFQLASTSSSIDGAAGILGIGDKNTEASEQQYDDLPWALQKAGKTPKASYSLYLGSGDGASGSVIFGGIDTEKYSGELTKYPVDTSNGPGLFVNVATFSVDGKDFSTDAPFLLDSGTSLGFVPQDVQDYLDSIFNPTMVQEGAITYRQVSCDQPTDKFFTLDFGQNKITFSYADAISQQDENTCLLGFTYHDDTYILGDVFLRNAYVYYDLTDGSISLAQAKYTSASNIVSA

SEQ ID NO: 27 - DNA, 1245 bp, Meyerozyma guilliermondii
  source 1..1245: mol_type = genomic DNA
atgttcccattcaatattgctgctgctcaagctttggttttggctttgatagttccatctactttgggtttgtccattccaattcaacctagagatgacaagaaggttattgccttggatttcaatgttactccaccacaaggtacaaacttcacttttaacggtgactcattgtccttgttggttgatggtaattccaagttgaaggttgccaactctccaatttctgttcctattcatggttctggtggttcctacatcatcgaattattgatcggttcctccaagaacaaggttactgttgctttggatactggttcctctgatttgaacgttgttgactctaattcttactgcggtgataactctgattgtcatgctaatggttcttacgacccatcttcttctactacctctcaaaacttgaacatcccagttgatttggaatatggttcaggtgcttctactggtacttactacaaagatgatgtctctttgggtgaagcttctggtgttactgttaagggtttacaattcgccgatatcactgattctgaaggtgcttcaggtattttgggtattggttacgaagctaatgaagctgctagacaagaataccctaacttcgtttctttgttgaagtcccaaaactacatcaacaagagagcctactccttgtacttgaattctgttaatgctcaaaccggtactatcttgtttggtggtaaagataccagaaagtacaaaggtaaattgaccacctactctacctctggtaacgaaagattgcaaatcccattgcaatcattgtctgttggtggtcaaagtgtttcattgtcaggtttgaatggtgttttggattctggtactaccttgtcctacattaagaaagaagaatattaccaattggccgctaagttgaactggatcgatgtttcttcttctgctcaacaaccaccaggtaattactatgctggtccatgttctggtccagattttgtttttaactttggtaacggtggtactgttaccgttccatattctgctgttactatgtctgttaccgatgacaactccttgtgcattattactgttttgccaaacaccaacccaaagattgaaggtattacaatcttgggtgacaacttcttgagatccgcttatgttgtttacgatttggatgccgaacaaatctctattgccaacgttaactacaccaccgattccaatgttgtcgaattgtaa

SEQ ID NO: 28 - protein, 414 aa, Meyerozyma guilliermondii
MFPFNIAAAQALVLALIVPSTLGLSIPIQPRDDKKVIALDFNVTPPQGTNFTFNGDSLSLLVDGNSKLKVANSPISVPIHGSGGSYIIELLIGSSKNKVTVALDTGSSDLNVVDSNSYCGDNSDCHANGSYDPSSSTTSQNLNIPVDLEYGSGASTGTYYKDDVSLGEASGVTVKGLQFADITDSEGASGILGIGYEANEAARQEYPNFVSLLKSQNYINKRAYSLYLNSVNAQTGTILFGGKDTRKYKGKLTTYSTSGNERLQIPLQSLSVGGQSVSLSGLNGVLDSGTTLSYIKKEEYYQLAAKLNWIDVSSSAQQPPGNYYAGPCSGPDFVFNFGNGGTVTVPYSAVTMSVTDDNSLCIITVLPNTNPKIEGITILGDNFLRSAYVVYDLDAEQISIANVNYTTDSNVVEL

SEQ ID NO: 29 - DNA, 1743 bp, Clavispora lusitaniae
  source 1..1743: mol_type = genomic DNA
atgaagttcttgtccttggttactttggctgctgctgtttctggtgctactgttgaaaatttgagaagagaagaaaacaagcaagaaaccatcgtcccattgagattggatttctctgttttgagaggttcctcaccacaagatatggctccaggtagaggtgctgctttggctaaaagagatggtcaagctgaattgaccatccaaaacgaacaaacttactactccgctgatttgaagttgggttctgatcatcaagaagtttccgttttggttgataccggttcttctgatttgtggattatggcttctgatgtcgaatgctactcttcccaatcacaatcttcatctaccaagagatcagttggtgatcatttcggtagaagaagagctttgtctgaagatgatttggctcatgctttgttccaagaacaatctgataataccccagatgcttctcaaccattgcaagataagagagatactgaatctatggccttcccagatattgcctctattttggaatccttcaccatcatcgaaactaacattcctcaaccatctggttcttcatctccagatgtttcaggtggttctggtggtagtggtggttacggtggttctaatacttgtacttctgaaggttccttcaacaccgattcctctgatactttccatatgaattcttccgctccagatttcgctattcaatatgctgatggtacttctgctagaggtttttggggtactgattacgtttctattgataccgctaacgtcagtgatgtttctttcgctgttgttaacgaaaccgattctggttttggtgttttgggtattggtttgccaggtttggaaactacttattctggtacttcaggttcctacatgtacgaaaatttccctatgagattgaagtcctccggtgttattcacaagaacgtctactcattatacttgaacaaggctgatgctcaatccggttctgttttgtttggtggtgttgatcatgctaagtacactggtcaattgactactgttccattggtcaacatctactccaagtactacaagaacccaatcagattggatgttgccttggattccatttctttcgaatctacctcttctaacattaccgcttacaagggtaatttggcagctttgttggattcaggtactacctattcttacttgccaacctctgttttcgaaagattcatcaacgttgtcaacgcccaatcttcttcaattggtttgtaccaattgtcctgctcctacaatactgattctgcttctgttgttttcaacttctctggtgctcaaatcaaggttccattgtctgatttggttatgacctacagaaacagatgctacttgaccgttttggaacaatcctctagttcctcttcatcctcatcatcttctactccagaatacgctgttttaggtgacaatttcttgagaaacgcctacgttgtttacaatttggacgactacgaaatctctttggctcaagctaagtataccgacgaagaagatatcgaaatcgtcagttcttctgttccatctgctgttaaggctggtggttattcttctacttctttgtccgaatcttccgatacctctgaagttactactttgtcctcatcctccttgaaaaaatcaggtgctccaagattagctccatggaaagaaatgggtgctgcattgatggttttgttggcttttgctttgatgtaa

SEQ ID NO: 30 - protein, 580 aa, Clavispora lusitaniae
MKFLSLVTLAAAVSGATVENLRREENKQETIVPLRLDFSVLRGSSPQDMAPGRGAALAKRDGQAELTIQNEQTYYSADLKLGSDHQEVSVLVDTGSSDLWIMASDVECYSSQSQSSSTKRSVGDHFGRRRALSEDDLAHALFQEQSDNTPDASQPLQDKRDTESMAFPDIASILESFTIIETNIPQPSGSSSPDVSGGSGGSGGYGGSNTCTSEGSFNTDSSDTFHMNSSAPDFAIQYADGTSARGFWGTDYVSIDTANVSDVSFAVVNETDSGFGVLGIGLPGLETTYSGTSGSYMYENFPMRLKSSGVIHKNVYSLYLNKADAQSGSVLFGGVDHAKYTGQLTTVPLVNIYSKYYKNPIRLDVALDSISFESTSSNITAYKGNLAALLDSGTTYSYLPTSVFERFINVVNAQSSSIGLYQLSCSYNTDSASVVFNFSGAQIKVPLSDLVMTYRNRCYLTVLEQSSSSSSSSSSSTPEYAVLGDNFLRNAYVVYNLDDYEISLAQAKYTDEEDIEIVSSSVPSAVKAGGYSSTSLSESSDTSEVTTLSSSSLKKSGAPRLAPWKEMGAALMVLLAFALM

SEQ ID NO: 31 - DNA, 1635 bp, Candida albicans
  source 1..1635: mol_type = genomic DNA
atgagattgaactccgttgccttgttgtctttggttgctactgctttggctgctaaagctccattcaagattgatttcgaagtcagaagaggtgaatccaaggatgatttgtctccagaagatgattctaacccaagattcgttaagagagatggttctttggatatgaccttgactaacaagcaaaccttctacatggctaccttgaagattggttctaacgaagatgaaaacagagtcttggaagatactggttcttctgatttgtgggttatgtcccatgatttgaagtgtgtttctgccccaatttccaagagaaacgaaagatcatttggtcatggtactggtgtcaagttgaacgaaagagaattgatgcaaaagagaaagaacttgtaccaaccatccagaaccattgaaaccgacgaagaaaaagaagcctctgaaaagatccacaacaagttgtttggtttcggttctatctactccaccgtttacattactgaaggtccaggtgcttactctactttttctccattggtcggtactgaaggtggttcaggtggtagtggtggttctaatacttgtagatcttacggttctttcaacaccgaaaactctgataccttcaaaaagaacaacaccaacgactttgaaatccaatacgctgatgatacctccgctattggtatttggggttacgatgatgttaccatctctaacgttaccgttaaggatttgtctttcgctattgctaacgaaacttcctctgatgttggtgttttgggtattggtttgccaggtttggaagttactacacaattgagatacacctaccaaaacttgccattgaagttgaaggccgatggtattattgccaagtccttgtactccttgtatttgaatactgctgatgctaaggccggttctattttgtttggtgctattgatcatgccaagtaccaaggtgacttggttactgttaagatgatgagaacctactcccaaatctcttacccagttagaattcaagttccagtcttgaagatcgatgttgaatcttcttcaggttccaccaccaatattttgtctggtactacaggtgttgttttggatacaggttctaccttgtcttacgttttttccgataccttgcaatctttgggtaaggctttgaatggtcaatactctaattctgttggtgcctacgttgttaactgtaacttggctgattcttctagaaccgttgatattgaattcggtggtaacaagaccatcaaggttccaatttccgatttggttttacaagcctctaagtctacctgcattttgggtgttatgcaacaatcctcttcctcctcttacatgttgttcggtgataacattttgagatccgcctacatcgtttacgatttggatgattacgaagtctccttggctcaagtttcttacaccaacaaagaatccatcgaagttattggtgcttccggtattactaattcttctggttctggtacaacctcctcttctggtacttctacatctacttctactagacattccgctggttccattatctctaatccagtttatggtttgttgttgtccttgttgatctcctactacgttttggtctaa

SEQ ID NO: 32 - protein, 544 aa, Candida albicans
MRLNSVALLSLVATALAAKAPFKIDFEVRRGESKDDLSPEDDSNPRFVKRDGSLDMTLTNKQTFYMATLKIGSNEDENRVLEDTGSSDLWVMSHDLKCVSAPISKRNERSFGHGTGVKLNERELMQKRKNLYQPSRTIETDEEKEASEKIHNKLFGFGSIYSTVYITEGPGAYSTFSPLVGTEGGSGGSGGSNTCRSYGSFNTENSDTFKKNNTNDFEIQYADDTSAIGIWGYDDVTISNVTVKDLSFAIANETSSDVGVLGIGLPGLEVTTQLRYTYQNLPLKLKADGIIAKSLYSLYLNTADAKAGSILFGAIDHAKYQGDLVTVKMMRTYSQISYPVRIQVPVLKIDVESSSGSTTNILSGTTGVVLDTGSTLSYVFSDTLQSLGKALNGQYSNSVGAYVVNCNLADSSRTVDIEFGGNKTIKVPISDLVLQASKSTCILGVMQQSSSSSYMLFGDNILRSAYIVYDLDDYEVSLAQVSYTNKESIEVIGASGITNSSGSGTTSSSGTSTSTSTRHSAGSIISNPVYGLLLSLLISYYVLV

SEQ ID NO: 33 - DNA, 1515 bp, Meyerozyma guilliermondii
  source 1..1515: mol_type = genomic DNA
atgaagttgaccgtcatcttgatcattatcgcctctcatttggttgctgctactttgaaattggatttcgccattcatagaggtaactccaaagatgatatggccttggaaaatgaaccacaattcgtcaagagatccgaagatgataagttcactatggaattgactaacaaggccactttctacgttgccgaattgaaaattggttccaacaaggatcaagttggtgttttggttgataccggttcttctgatttggttgtcttgtctgaagatttgaagtgctacagaagatcctacaagaagagagaagaagaaaagttggtttccgactccttctacgaatacgaaactgtttctgcttacttgtctgatttcggtggttatgataccttgtacgattctgaaactaccacctatgattccgaaaccacttatgatttgccaactactgatacctgtacctcttatggttcattctctaccgaagattccgattctttttctgttaatggttccgctccagacttcttcatttcttatgctgatggtactggtgcttctggtgtttggggtactgatgaagtagaaattggtaacgctaccgtcaactctttgtcttttgctgttgctaacaagtctaccactaacgttggtattttgggtattggtttggctggtttggaaaccactaagaacatttctactggtgaaggttacgaatactccaacttgccattgatgttgaagtcccaaggtattattgataaggccttgtactccttgtatttgggtaacatctctgatagacacggttctgttttgttcggtgctattgataatgctaagtacaccggtaacttgaaaaccgttaagatggtcaactcctacagaaatgtttccaacgttccattgagaatcgaagttgaaaactccgccattaaggttgatgatggtgaatccatttccgacgtttcttacgaatctatcccagttttgttggatacaggtgctacttattctcacttgtaccattccattttgaccgacatcgtttctaaattgggtggttcctactcattatactctgacttgtataccgttccatgctcatctgaaggttctattactttcgaattctccggtcaagaaatcgccgttagaatgcaagatttgatcaagcaatctgaagatggtacttgcatcttgactttgttgccacaacctacttctaacccttatatgtctttgggtgacaacttcttgagacatgcttacgttgtttgcgacttggaaaactacgaagtttctttggctccaatcaagtactcctccgatgaagatatcgaagttatcacttcttccgtcccaactaagtctccaattcaagcttctaagtctaactcccatgcttcttcagctcatggtaaatctaaatctgcagctggtgtttcttcttacggtttgccatctttgtctcaagttgtattgggtttggtcgtcatgttcttgtaa

SEQ ID NO: 34 - protein, 504 aa, Meyerozyma guilliermondii
MKLTVILIIIASHLVAATLKLDFAIHRGNSKDDMALENEPQFVKRSEDDKFTMELTNKATFYVAELKIGSNKDQVGVLVDTGSSDLVVLSEDLKCYRRSYKKREEEKLVSDSFYEYETVSAYLSDFGGYDTLYDSETTTYDSETTYDLPTTDTCTSYGSFSTEDSDSFSVNGSAPDFFISYADGTGASGVWGTDEVEIGNATVNSLSFAVANKSTTNVGILGIGLAGLETTKNISTGEGYEYSNLPLMLKSQGIIDKALYSLYLGNISDRHGSVLFGAIDNAKYTGNLKTVKMVNSYRNVSNVPLRIEVENSAIKVDDGESISDVSYESIPVLLDTGATYSHLYHSILTDIVSKLGGSYSLYSDLYTVPCSSEGSITFEFSGQEIAVRMQDLIKQSEDGTCILTLLPQPTSNPYMSLGDNFLRHAYVVCDLENYEVSLAPIKYSSDEDIEVITSSVPTKSPIQASKSNSHASSAHGKSKSAAGVSSYGLPSLSQVVLGLVVMFL

SEQ ID NO: 35 - DNA, 1566 bp, Bacillus subtilis
  source 1..1566: mol_type = genomic DNA
atgggtttgggtaagaaattgtctgttgctgttgctgcttctttcatgtctttgactatttctttgccaggtgttcaagctgctgaaaacccacaattgaaagaaaacttgaccaacttcgttccaaagcactctttggttcaatctgaattgccatccgtttctgataaggccattaagcaatacttgaagcaaaacggtaaggttttcaagggtaacccatctgaaagattgaagttgattgatcaaaccaccgatgacttgggttacaagcactttagatatgttccagttgttaacggtgttccagtcaaggattcccaagttattatccacgttgacaagtccaacaacgtttacgctattaacggtgaattgaacaacgatgtttctgctaagactgccaactctaaaaagttgtctgctaatcaagctttggatcatgcttacaaggccattggtaaatcacctgaagctgtttctaatggtactgttgctaacaagaacaaggctgaattgaaagctgctgctactaaggatggtaaatacagattggcttacgatgtcaccatcagatatattgaaccagaaccagctaactgggaagttactgttgatgctgaaactggtaagatcttgaaaaagcaaaacaaggttgaacatgctgctacaactggtactggtactactttgaaaggtaagaccgtttccttgaacatctcttccgaatctggtaagtacgttttgagagatttgtctaagccaaccggtactcaaattatcacttacgacttgcaaaacagagaatacaacttgccaggtactttggtttcttctaccaccaatcaattcactacctcttcacaaagagctgctgttgacgctcattacaatttgggtaaagtttacgactacttctaccaaaagttcaacagaaactcctacgataacaagggtggtaagattgtctcttctgttcattacggttccagatacaacaatgctgcttggattggtgatcaaatgatctatggtgacggtgatggttcttttttctctccattgtctggttctatggatgttactgctcacgaaatgactcatggtgttactcaagaaactgctaacttgaactacgaaaatcaaccaggtgccttgaacgaatctttctctgatgtttttggttacttcaacgacaccgaagattgggatattggtgaagatattaccgtttctcaaccagccttgagatctttgtcaaatccaactaagtatggtcaaccagataacttcaagaactacaagaacttgccaaacactgatgctggtgattatggtggtgttcataccaattctggtattccaaacaaagctgcctacaacaccattaccaaaatcggtgttaacaaggccgaacaaatctactatagagctttgactgtttacttgaccccatcttctactttcaaagatgctaaagccgccttgattcaatctgctagagacttgtatggttctcaagatgctgcttcagttgaagctgcatggaatgctgttggtttgtga

SEQ ID NO: 36 - protein, 521 aa, Bacillus subtilis
MGLGKKLSVAVAASFMSLTISLPGVQAAENPQLKENLTNFVPKHSLVQSELPSVSDKAIKQYLKQNGKVFKGNPSERLKLIDQTTDDLGYKHFRYVPVVNGVPVKDSQVIIHVDKSNNVYAINGELNNDVSAKTANSKKLSANQALDHAYKAIGKSPEAVSNGTVANKNKAELKAAATKDGKYRLAYDVTIRYIEPEPANWEVTVDAETGKILKKQNKVEHAATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 37 - DNA, 1185 bp, Candida tropicalis
  source 1..1185: mol_type = genomic DNA
atgttcttgtcccaattggtcgttttcttggttttcggtttgttggttactgcttctccaactacttctccaccaggttttatttctttggacttcgttatcatcaagacccaaaagaacatcgtcccaaacgaaaacatcatcgtcagtaaaagacaaccagttccagtcaccttgatcaaagaacaaattgcttacgctgccgaaattaccatcggttctaacaatcaaaagcaaaccgtcattatcgacaccggttcttcagatttgtgggttgttgataagaacgctacctgtgttagaagattcgaacaacaagttcaagatttctgcaaggctaacggtacttacgatccaattacttcttcatccgctaagaagttgggtactgttttcgatatttcctacggtgataagaccaactcttctggtaattggtacaaggataccattaagattggtggtatcaccatcaccaatcaacaattcgctaacgttaagtctacctctgttgctcaaggtgttatgggtattggtttcaagactaacgaagcttctaacgttacctacgataacgttccaattaccttgaagaagcaaggtatcatttccaagtctgcctactccttgtacttgaattcttctgattctaccaccggtgaaattatctttggtggtgttgataacgctaagtacaccggtaaattgatcgatttgccagttacctctaacagagaattgagaatctacttgaactccttgaccattggtgttaccaacatttctgcttccatggatgttttgttggattctggtactaccttctcatacttgcaacaagatgtcttgcaacacgttgtcgataagtttaacggtcaattgattcatgatgccttgggtaatccattgcatttggttgattgtgacttgccaggtaacatcgatttcgaattctctaactcctccaagatctctgttccatcttctgaatttgccgttaagttgtacactatcaacggtgaattatacccaaagtgccaattgtctatcttgacctcttctgctaacatcttgggtaacaatttcttgagatccgcctacatcgtttacgacttggaagataagaaaatctccttggcccaagtcaagtacacttctaagtctaacattttgccattgacttaa

SEQ ID NO: 38 - protein, 394 aa, Candida tropicalis
MFLSQLVVFLVFGLLVTASPTTSPPGFISLDFVIIKTQKNIVPNENIIVSKRQPVPVTLIKEQIAYAAEITIGSNNQKQTVIIDTGSSDLWVVDKNATCVRRFEQQVQDFCKANGTYDPITSSSAKKLGTVFDISYGDKTNSSGNWYKDTIKIGGITITNQQFANVKSTSVAQGVMGIGFKTNEASNVTYDNVPITLKKQGIISKSAYSLYLNSSDSTTGEIIFGGVDNAKYTGKLIDLPVTSNRELRIYLNSLTIGVTNISASMDVLLDSGTTFSYLQQDVLQHVVDKFNGQLIHDALGNPLHLVDCDLPGNIDFEFSNSSKISVPSSEFAVKLYTINGELYPKCQLSILTSSANILGNNFLRSAYIVYDLEDKKISLAQVKYTSKSNILPLT

SEQ ID NO: 39 - DNA, 1173 bp, Saccharomycopsis fibuligera
  source 1..1173: mol_type = genomic DNA
atgttgttctccaagtccttgttgttgtctgttttggcttctttgtcttttgctgctccagttgaaaagagagaaaagactttgaccttggatttcgacgtcaagagaatttcttctaaggctaagaacgttaccgttgcttcttcaccaggtttcagaagaaacttgagagctgcttctgatgctggtgttactatttctttggaaaacgaatactccttctacttgaccaccattgaaattggtactccaggtcaaaagttgcaagttgatgttgatactggttcctctgatttgtgggttcctggtcaaggtacttcttcattatatggtacttacgaccataccaagtccacctcttacaaaaaagatagatccggtttctccatctcttacggtgatggttcttctgctagaggtgattgggctcaagaaactgtttctattggtggtgcttctattaccggtttggaatttggtgatgctacctctcaagatgtaggtcaaggtttgttgggtattggtttgaaaggtaatgaagcttccgctcaatcttctaactctttcacttacgataacttgccattgaagttgaaggaccaaggtttgattgataaggctgcctactccttgtatttgaactctgaagatgctaccagtggttccattttgtttggtggttctgattcctctaagtactctggttctttggctactttggatttggttaacatcgatgatgaaggtgactctacttctggtgctgttgctttttttgttgaattggaaggtattgaagccggttcttcctctattaccaaaactacttatccagccttgttggattctggtactactttgatatatgccccatcttctattgcctcttccattggtagagaatacggtacttattcctactcatatggtggttacgttacctcttgtgatgcaactggtccagattttaagttctccttcaacggtaaaaccattaccgttccattctccaacttgttgttccaaaattccgaaggtgattccgaatgtttggttggtgttttatcttccggttccaactactacattttgggtgatgcttttttgagatccgcctacgtttactacgatatcgataattcccaagttggtattgctcaagccaagtactaa

SEQ ID NO: 40 - protein, 390 aa, Saccharomycopsis fibuligera
MLFSKSLLLSVLASLSFAAPVEKREKTLTLDFDVKRISSKAKNVTVASSPGFRRNLRAASDAGVTISLENEYSFYLTTIEIGTPGQKLQVDVDTGSSDLWVPGQGTSSLYGTYDHTKSTSYKKDRSGFSISYGDGSSARGDWAQETVSIGGASITGLEFGDATSQDVGQGLLGIGLKGNEASAQSSNSFTYDNLPLKLKDQGLIDKAAYSLYLNSEDATSGSILFGGSDSSKYSGSLATLDLVNIDDEGDSTSGAVAFFVELEGIEAGSSSITKTTYPALLDSGTTLIYAPSSIASSIGREYGTYSYSYGGYVTSCDATGPDFKFSFNGKTITVPFSNLLFQNSEGDSECLVGVLSSGSNYYILGDAFLRSAYVYYDIDNSQVGIAQAKY

SEQ ID NO: 41 - DNA, 1056 bp, Ananas comosus
  source 1..1056: mol_type = genomic DNA
atggcctctaaggttcaattggttttcttgtttttgttcttgtgcgctatgtgggcttctccatctgctgcttctagagatgaacctaatgatccaatgatgaagagattcgaagaatggatggctgaatacggtagagtttacaaagatgacgacgaaaagatgagaagattccaaatcttcaagaacaacgtcaagcacatcgaaaccttcaactctagaaacgaaaactcttacaccttgggtatcaatcaattcaccgatatgaccaagtctgaattcgttgctcaatacactggtgtttctttgccattgaacatcgaaagagaaccagttgtttccttcgatgatgtcaacatttctgctgttccacaatccattgattggagagattatggtgctgttaacgaagtcaagaatcaaaacccatgtggttcttgttggtcttttgctgctattgctactgttgaaggtatctacaagattaagaccggttacttggtcagtttgtccgaacaagaagttttggattgcgctgtttcttacggttgtaaaggtggttgggttaacaaagcctacgatttcattatctccaacaacggtgttaccaccgaagaaaattatccatacttggcttaccaaggtacttgcaatgctaattcttttccaaactccgcttacatcactggttactcttacgttagaagaaacgacgaaagatccatgatgtacgccgtttctaatcaacctattgctgctttgattgacgcctctgaaaacttccaatattacaacggtggtgttttctctggtccatgtggtacatctttgaaccatgccattaccattattggttacggtcaagattcttccggtactaagtattggatcgttagaaattcttggggttcttcatggggtgaaggtggttatgttagaatggctagaggtgtttcatcttcctctggtgtttgtggtattgctatggctcctttgtttccaactttacaatctggtgctaacgccgaagttatcaagatggtttctgaaacctaa

SEQ ID NO: 42 - protein, 351 aa, Ananas comosus
MASKVQLVFLFLFLCAMWASPSAASRDEPNDPMMKRFEEWMAEYGRVYKDDDEKMRRFQIFKNNVKHIETFNSRNENSYTLGINQFTDMTKSEFVAQYTGVSLPLNIEREPVVSFDDVNISAVPQSIDWRDYGAVNEVKNQNPCGSCWSFAAIATVEGIYKIKTGYLVSLSEQEVLDCAVSYGCKGGWVNKAYDFIISNNGVTTEENYPYLAYQGTCNANSFPNSAYITGYSYVRRNDERSMMYAVSNQPIAALIDASENFQYYNGGVFSGPCGTSLNHAITIIGYGQDSSGTKYWIVRNSWGSSWGEGGYVRMARGVSSSSGVCGIAMAPLFPTLQSGANAEVIKMVSET

SEQ ID NO: 43 - DNA, 642 bp, Ananas comosus
  source 1..642: mol_type = genomic DNA
atggctgttccacaatctatcgattggagagattatggtgctgttacctccgttaagaatcaaaatccatgtggtgcttgttgggcttttgctgctattgctactgttgaatctatctacaagatcaagaagggtatcttggaaccattgtccgaacaacaagttttggattgtgctaaaggttacggttgtaaaggtggttgggaattcagagctttcgaattcattatctccaacaagggtgttgcttctggtgctatctatccatacaaagctgcaaaaggtacttgtaagactgatggtgttccaaactctgcttacattactggttatgctagagtcccaagaaacaacgaatcttctatgatgtacgccgtttccaagcaacctattactgttgctgttgatgctaacgctaacttccaatattacaagtccggtgtttttaacggtccttgtggtacttctttgaaccatgctgttactgctattggttacggtcaagattctatcatctacccaaaaaaatggggtgctaagtggggtgaagctggttatattagaatggctagagatgtctcttcctcctctggtatttgtggtattgctattgatccattatacccaaccttggaagaataa

SEQ ID NO: 44 - protein, 213 aa, Ananas comosus
MAVPQSIDWRDYGAVTSVKNQNPCGACWAFAAIATVESIYKIKKGILEPLSEQQVLDCAKGYGCKGGWEFRAFEFIISNKGVASGAIYPYKAAKGTCKTDGVPNSAYITGYARVPRNNESSMMYAVSKQPITVAVDANANFQYYKSGVFNGPCGTSLNHAVTAIGYGQDSIIYPKKWGAKWGEAGYIRMARDVSSSSGICGIAIDPLYPTLEE

SEQ ID NO: 45 - DNA, 1131 bp, unidentified
  misc_feature 1..1131: note = zea mays Vignain like
  source 1..1131: mol_type = genomic DNA
atggctagagctttgcctttggttttggctgcttctgttttgttggctgctgctttgttgttattagcttttgctccagctccagctgctgctgttgattttggtgctgaagatttggcttctgaagaagctttgtgggcattatacgaaagatggagaggtagacatgctttggcaagagatttgggtgataaggctagaagattcaacgttttcaaggccaacgttagattgatccacgaattcaacagaagagatgaaccatacaagttgagattgaacagattcggtgatatgaccgctgatgaattcagaagacattacgctggttctagagttgctcatcatagaatgtttagaggtgacagacaaggttcttctgcttcagcttcttttatgtatgctgatgctagagatgttccagcttcagttgattggagacaaaaaggtgctgttaccgatgttaaggatcaaggtcaatgtggttcttgttgggctttttctactattgctgcagttgaaggtattaacgccattaagactaagaacttgacctccttgtctgaacaacaattggttgattgtgataccaaggctaatgctggttgtaatggtggtttgatggattacgcctttcaatatatcgctaaacatggtggtgttgctgcagaagatgcttatccatatagagctagacaagcctcttgtaaaaaatctcctgctccagttgttaccattgacggttatgaagatgttcctgctaatgatgaatccgctttgaaaaaagctgttgcccatcaaccagtttccgttgctattgaagcttctggttctcatttccaattctactctgaaggtgttttctctggtagatgtggtactgaattggatcatggtgttactgctgttggttacggtgttacagcagatggtactaagtattggttggttaagaattcatggggtccagaatggggtgaaaaaggttatattagaatggccagagatgttgctgccaaagaaggtcattgcggtattgcaatggaagcttcttatccagttaagacttctccaaacccaaaggttcatgctgttgttgatgaagatggttcttcccatgatgaattgtga

SEQ ID NO: 46 - protein, 376 aa, unidentified
  REGION 1..376: note = zea mays Vignain like
MARALPLVLAASVLLAAALLLLAFAPAPAAAVDFGAEDLASEEALWALYERWRGRHALARDLGDKARRFNVFKANVRLIHEFNRRDEPYKLRLNRFGDMTADEFRRHYAGSRVAHHRMFRGDRQGSSASASFMYADARDVPASVDWRQKGAVTDVKDQGQCGSCWAFSTIAAVEGINAIKTKNLTSLSEQQLVDCDTKANAGCNGGLMDYAFQYIAKHGGVAAEDAYPYRARQASCKKSPAPVVTIDGYEDVPANDESALKKAVAHQPVSVAIEASGSHFQFYSEGVFSGRCGTELDHGVTAVGYGVTADGTKYWLVKNSWGPEWGEKGYIRMARDVAAKEGHCGIAMEASYPVKTSPNPKVHAVVDEDGSSHDEL

SEQ ID NO: 47 - DNA, 1413 bp, unidentified
  misc_feature 1..1413: note = zea mays cysteine protease
  source 1..1413: mol_type = genomic DNA
atggctgctttgggtagaggtttgcctttgttgttgttgttgttattattggctgtttccggtgctgctaatgctgctgctgcaccaggtggtatgtctattattacttacaacgaagaacatggtgccagaggtttggaaagaactgaacctgaagttagagctatgtacgatttgtggttggctgaacatggtagagcttataatgctttaggtgaaggtgaaggtgaaagagacagaagatttttggttttctgggacaacttgagattcgttgatgctcataacgaaagagctggtgctagaggttttagattgggtatgaatcaattcgccgatttgaccaacgatgaattcagagctgcttatttgggtgctatggttccagctgctagaagaggtgctgttgttggtgaaagatatagacatgatggtgctgctgaagaattgccagaatctgttgattggagagaaaaaggtgcagttgctccagttaagaatcaaggtcaatgtggttcttgttgggctttttctgctgtttcttctgtcgaatccgttaatcaaatcgttaccggtgaaatggttaccttgtctgaacaagaattggttgaatgttctaccgatggtggtaattctggttgtaatggtggtttaatggatgctgccttcgatttcattattaagaacggtggtattgacaccgaagatgattatccatatagagccgttgatggtaaatgcgacatgaatagaaagaacgccagagttgtttccattgacggttttgaagatgttccagaaaacgacgaaaagtccttgcaaaaagctgttgctcatcaaccagtttccgttgctattgaagccggtggtagagaatttcaattatacaagtctggtgttttctccggttcttgtactaccaatttggatcatggtgttgttgcagttggttacggtgctgaaaatggtaaagattactggatcgttagaaactcttggggtccaaaatggggtgaagctggttatattagaatggaaagaaacgttaacgcctctactggtaaatgtggtattgctatgatggcttcttacccaactaagaaaggtgctaatccaccaagaccatctccaacaccaccaactccaccagctgctccagataatgtttgtgacgaaaatttctcttgctctgctggttctacatgttgttgtgcttttggtttcagaaacgtttgtttggtttggggttgttgtccagttgaaggtgctacttgttgtaaagatcatgcttcatgttgtccaccaggttatccagtatgtaatgttagagctggtacttgctctgtctctaagaattctccattgtctgttaaggctttgaagagaactttggctaagttgtctactgcctga

SEQ ID NO: 48 - protein, 470 aa, unidentified
  REGION 1..470: note = zea mays cysteine protease
MAALGRGLPLLLLLLLLAVSGAANAAAAPGGMSIITYNEEHGARGLERTEPEVRAMYDLWLAEHGRAYNALGEGEGERDRRFLVFWDNLRFVDAHNERAGARGFRLGMNQFADLTNDEFRAAYLGAMVPAARRGAVVGERYRHDGAAEELPESVDWREKGAVAPVKNQGQCGSCWAFSAVSSVESVNQIVTGEMVTLSEQELVECSTDGGNSGCNGGLMDAAFDFIIKNGGIDTEDDYPYRAVDGKCDMNRKNARVVSIDGFEDVPENDEKSLQKAVAHQPVSVAIEAGGREFQLYKSGVFSGSCTTNLDHGVVAVGYGAENGKDYWIVRNSWGPKWGEAGYIRMERNVNASTGKCGIAMMASYPTKKGANPPRPSPTPPTPPAAPDNVCDENFSCSAGSTCCCAFGFRNVCLVWGCCPVEGATCCKDHASCCPPGYPVCNVRAGTCSVSKNSPLSVKALKRTLAKLSTA

SEQ ID NO: 49 - DNA, 1392 bp, unidentified
  misc_feature 1..1392: note = zea mays cysteine protease 1(2)
  source 1..1392: mol_type = genomic DNA
atggctgcttctactacagctgctgctgctttgttgttgttgttattgtctttggctgctgctgcagatatgtctatagtttcttatggtgaaagatccgaagaagaagctagaagaatgtatgctgaatggatggctgctcatggtagaacttataatgctgttggtgaagaagaaagaagataccaagttttcagagacaacttgagatatatcgatgctcataatgctgcagctgatgctggtgttcattcttttagattgggtttgaacagattcgccgatttgactaacgatgaatacagagctacttacttgggtgctagaactagaccacaaagagaaagaaaattgggtgcaagatatcatgctgccgataatgaagatttgccagaatctgttgattggagagctaaaggtgctgttgcagaagttaaggatcaaggttcttgtggttcatgttgggctttttctactattgctgctgtcgaaggtatcaatcaaatcgttactggtgacttgatctccttgtctgaacaagaattggttgattgcgacacctcttacaatcaaggttgtaatggtggtttgatggattacgccttcgaattcattattaacaacggtggtattgacaccgaaaaggattatccatacaaaggtactgatggtagatgcgacgttaatagaaagaacgctaaggttgttaccatcgactcttatgaagatgttccagctaatgacgaaaagtccttgcaaaaagctgttgctaatcaaccagtttccgttgctattgaagctgctggtactgcttttcaattatactcctctggtattttcactggttcttgcggtacagctttggatcatggtgttacagctgttggttatggtactgaaaatggtaaggattactggatcgttaagaactcttggggttcttcttggggtgaatctggttatgttagaatggaaagaaacatcaaggcctcttcaggtaaatgtggtattgctgttgaaccatcctacccattgaaagaaggtgctaatccaccaaatccaggtccatctccaccatcaccaactccagctccagctgtttgtgataattactattcttgtccagattccaccacctgttgttgtatctatgaatacggtaaatactgcttcgcttggggttgttgtccattggaaggtgcaacttgttgtgatgatcattactcatgttgcccacatgattacccaatctgtaatgttagacaaggtacttgcttgatgggtaaagattctccattgtccttatctgttaaggctactaagagaactttggctaaaccacattgggctttctctggtaatactgctgatggtatgaagtcctctgcttga

SEQ ID NO: 50 - protein, 463 aa, unidentified
  REGION 1..463: note = zea mays cysteine protease 1(2)
  VARIANT 34: note = Xaa can be any naturally occurring amino acid
MAASTTAAAALLLLLLSLAAAADMSIVSYGERSXEEARRMYAEWMAAHGRTYNAVGEEERRYQVFRDNLRYIDAHNAAADAGVHSFRLGLNRFADLTNDEYRATYLGARTRPQRERKLGARYHAADNEDLPESVDWRAKGAVAEVKDQGSCGSCWAFSTIAAVEGINQIVTGDLISLSEQELVDCDTSYNQGCNGGLMDYAFEFIINNGGIDTEKDYPYKGTDGRCDVNRKNAKVVTIDSYEDVPANDEKSLQKAVANQPVSVAIEAAGTAFQLYSSGIFTGSCGTALDHGVTAVGYGTENGKDYWIVKNSWGSSWGESGYVRMERNIKASSGKCGIAVEPSYPLKEGANPPNPGPSPPSPTPAPAVCDNYYSCPDSTTCCCIYEYGKYCFAWGCCPLEGATCCDDHYSCCPHDYPICNVRQGTCLMGKDSPLSLSVKATKRTLAKPHWAFSGNTADGMKSSA

SEQ ID NO: 51 - DNA, 1176 bp, Candida dubliniensis
  source 1..1176: mol_type = genomic DNA
atgtttttgaagaacatcttcattgctttggccttcgctttgttggttgatgctactccagctaaaagatctgctggttttgttaccttggatttcgaagttattaagaccccagttaacgctactggtcaagatggtaaagttaagagacaagctattccagtcaccttgaacaacgaagttgtttcttatgctgccgatattaccgttggttctaacagacaaaagttcaacgttgttgttgacaccggttcttcagatttgtggattccagatgcttctgttacttgtgaaaatccaccaccaggtcaatctgctgatttctgtaaaggtaaaggtttgtacaccccaaaatcttctactacctctcaaagattgggtaacccattctatattggttacggtgatggttcttcttctcacggtacattatacaaggatactgttggttttggtggtgcctctattactaagcaagtttttgctgatgttaccaagacctccgttaatcaaggtattttgggtatcggttacaagactaatgaagctgctggtgattacgataacgttccagttactttgaagaaacaaggtgttattgctaagaacgcctactccttgtatttgaattctccaaatgctgctaccggtcaaattatcttcggtggtgttgataaggctaagtactctggttctttgattgctgttcctgttacctctgatagagaattgagaattaccttgaactccattaaggctgctggtaagaatatcaacggtaacattgatgtcttgttggactctggtactaccattacttactttcaacaagatgttgcccaaggtattatcgatgcttttcatgctgaattgaagcaagacggtaacggtaactcattatacgttgctgattgtcaaacctccggtactgttgattttaactttgctaacaacgctaagatctccgttccagcttctgaattcactgcttctttgttttacactaacggtcaaccatacccacaatgccaattgttgttgggtatcaacgatgctaacatcttgggtgacaatttcttgagatctgcctacatcgtttacgatttggatgacaacgaaatctctttggcccaagttaagtacacttccgcttctaatattgctgctttgacctaa

SEQ ID NO: 52 - protein, 391 aa, Candida dubliniensis
MFLKNIFIALAFALLVDATPAKRSAGFVTLDFEVIKTPVNATGQDGKVKRQAIPVTLNNEVVSYAADITVGSNRQKFNVVVDTGSSDLWIPDASVTCENPPPGQSADFCKGKGLYTPKSSTTSQRLGNPFYIGYGDGSSSHGTLYKDTVGFGGASITKQVFADVTKTSVNQGILGIGYKTNEAAGDYDNVPVTLKKQGVIAKNAYSLYLNSPNAATGQIIFGGVDKAKYSGSLIAVPVTSDRELRITLNSIKAAGKNINGNIDVLLDSGTTITYFQQDVAQGIIDAFHAELKQDGNGNSLYVADCQTSGTVDFNFANNAKISVPASEFTASLFYTNGQPYPQCQLLLGINDANILGDNFLRSAYIVYDLDDNEISLAQVKYTSASNIAALT

SEQ ID NO: 53 - DNA, 1233 bp, Candida orthopsilosis
  source 1..1233: mol_type = genomic DNA
atggtttccattaccaccatcatccaaaattccttgttgtcctctttgttcgccttgcattgtcaatctgctgctattattccaaactgcaatcaagttgttccagtcgatttccaagtttcctaccatgatagattcgatgacaaggatttgttcatcgacaacgaattcaacaaggatgaaggtttggctaagagagattacatcaagaccaacttgaagagaagaaagtcttactacaccaccgaattgaagatcggttctcaaaagaagaaggttaaggtcgttattgacaccggttcttcagatttgtgggttccatcttctgaagctaagtgtttggataactctaagtgcagatctgaaggtgtttactccgttgaaaaatctaagaccgccaagaagttgaatcaaccattcgaaattgaatacccagatgatacttctgcctctggtgtttatgttcaagataccgttagaatcggtaaaaagaccatctcccaacaacaatttgccgttgttgataagtcctctgctaaaatgggtgctttgggtattggtttgaagtctgatgaagaaacctctgacaactctacctacgataacttcgtctttaatttgaagaagcaaggtttgatcaacaaggccggttactcattatacttgccagaaaaagaaaagaagtccggtactattttgttcggtggtatcgatgaagataagtgctctggtaatttgaccaagttcatcgtttcctctgataagttggctgttccattgcaatctgtctcattcgaaaacaagaactacaagaacgataccgaagccattatcgattctggttctactttttcctacttgccaactaacatcgttgatggtatctccgatttgttgaacggttcttactcagacaaattgggtgtttacgttgttgactgcaagaacagaagaaagaagtctggttacatcaccttcaactttaacaacgaaacctccattttggccccattgtctcattttgttgattgcttgaaaaagatcaagtcccacgttccagatcacgaaaaaaatcattgcggtttgtccatcttgagaggtgatggtgacggtgaaactattttgggtgataactttttgagatccgcctacgtttctgttgacttggaagataagactgttggtttggctcaagtcaaacattctaagcaccattcctacaagggtatttaa

SEQ ID NO: 54 - protein, 410 aa, Candida orthopsilosis
MVSITTIIQNSLLSSLFALHCQSAAIIPNCNQVVPVDFQVSYHDRFDDKDLFIDNEFNKDEGLAKRDYIKTNLKRRKSYYTTELKIGSQKKKVKVVIDTGSSDLWVPSSEAKCLDNSKCRSEGVYSVEKSKTAKKLNQPFEIEYPDDTSASGVYVQDTVRIGKKTISQQQFAVVDKSSAKMGALGIGLKSDEETSDNSTYDNFVFNLKKQGLINKAGYSLYLPEKEKKSGTILFGGIDEDKCSGNLTKFIVSSDKLAVPLQSVSFENKNYKNDTEAIIDSGSTFSYLPTNIVDGISDLLNGSYSDKLGVYVVDCKNRRKKSGYITFNFNNETSILAPLSHFVDCLKKIKSHVPDHEKNHCGLSILRGDGDGETILGDNFLRSAYVSVDLEDKTVGLAQVKHSKHHSYKGI

SEQ ID NO: 55 - DNA, 1209 bp, Meyerozyma guilliermondii
  source 1..1209: mol_type = genomic DNA
atggtcagtttcactttggttactttcactgctttggctgctgctgtttttgctttggttgttccagaaaattcctcattggacaagagatcctctaacttcttgaagttggatttcgatgttgttagaaacgccgatgtcaagaaatccagagttcataagattgctaacggtaacggtacttttaccgaaaccttgtacaacaaggatgcctactacattacttacgtttacgctggttctaacaagcaaaagatcggtgttgatttggataccggttcttctgatttgtggttcgttgattcttctgctggttgttttgataacgcttgtcaatacggtacttacaacccattggaatctactacctctaagaacttgaacgaagttttcttcatcgaatacggtgataactcttacgctcaaggtttgtactacaccgatgatattggttttgcttcctctgattcatcagctgttgctaaaaacttgcaattcgctgatgctactagaaacgatgctggtatgggtattttgggtattggtttcgatactttgggtgctgaagctactgttattactggtggtccaacttatccaaacttgccatacgtcttgaaaaatcaaggtatcatctccaaggtcgcctactctttgtttttggattctccagatgctgcttctggttctgttttgtttggtggtaaagatttggctaaggtcaacggtgaattagttactttgccaattactaccgataacgccttgactgttaacttgaacactttgtccttgggtaatcaaaccgctgaaatcaatactgatgtcttgttggattctggtactaccattttctatttgccagatgccgctttcactcaattgattggttctttgccaggtgcttactggaaaaatgtttcaggtactccattatacttggttgattgtgctactccagttccagatttgactttcgaattcaacttcgaaggtatcactatcccagttccattgaacgatacttactctaccaacattaccgacgaaaacgatcaatttgttggttgcggttacttgatttccaccggtaacattttgggtgacacttttttgagaagagcctacgttgtttacgacttggaagatggtgaaatctctttgggtttgccaaagtatgctcaagaatctaacatcgtcccaatttaa

SEQ ID NO: 56 - protein, 402 aa, Meyerozyma guilliermondii
MVSFTLVTFTALAAAVFALVVPENSSLDKRSSNFLKLDFDVVRNADVKKSRVHKIANGNGTFTETLYNKDAYYITYVYAGSNKQKIGVDLDTGSSDLWFVDSSAGCFDNACQYGTYNPLESTTSKNLNEVFFIEYGDNSYAQGLYYTDDIGFASSDSSAVAKNLQFADATRNDAGMGILGIGFDTLGAEATVITGGPTYPNLPYVLKNQGIISKVAYSLFLDSPDAASGSVLFGGKDLAKVNGELVTLPITTDNALTVNLNTLSLGNQTAEINTDVLLDSGTTIFYLPDAAFTQLIGSLPGAYWKNVSGTPLYLVDCATPVPDLTFEFNFEGITIPVPLNDTYSTNITDENDQFVGCGYLISTGNILGDTFLRRAYVVYDLEDGEISLGLPKYAQESNIVPI

SEQ ID NO: 57 - DNA, 1239 bp, Scheffersomyces stipites
  source 1..1239: mol_type = genomic DNA
atggtcagtgttttgtctttcaccagacaagttttcttgaccttggctttgtctttgttggttaacgattctgttgccttggataagagatctccaggttttttgaagttggatttcactgttgctagaggtcaaggtaaagaatccttgaacttgacttctaccaacaagccatacttcgtttaccacgaaaaatctaagagagctactggtgaacctgaagttaccttgattaacgaaaagactttctacgccaccgacttggaaattggttctaacaaacaagctgttaccaccttgattgataccggttcttctgatttgtgggttgttgataagtctgctaagtgtcaagttactgaaaccggtcaatcttctacctactgttatgaatacggtgtttacgatcacaccaagtcctcttcatatcattctttgggttccaagttctccatcgaatatgttgatggtacttcttctactggtacttgggttaaggatgatgtttactttgctggtactactactggtgttacccaattacaattcggtgatgttactactacctcttctggttttggtgttttgggtattggttacgaagctaacgaatccactaataccgaatatccaaacttgccagtcttgttgaaaacccaaggtgttattgctaagaccgcctactcattatacttggctcaagaagatgctacctccggttctgttatttttggtggtattgatcaagccaagtactccggttcattgattactttgccagttacctcttcaagagaattggctattcatttgggtagtgttgccattaacggtaagactatttctgctaacttgaacccagttttggattccggtactactttgacttatttgccaactaagatcgttgcctctattgcttctgctttatctggtactgctgattcttcagttggttacatcatcaactgcaatcaaccatccaacaaatacttgaccttcaactttgattctggtgccactattaacgtcccattgtctgaattagctatcgacttgtacttgaccaacggtcaaaagtatcaatactgtgctttgggtgttttcgccacatcttctactccaattttgggtgataacttcttgagatctgcttacgttgcttacgatttggatgacaacactatttctttggcccaagttaagtacactaccgctgaaaacatagtcccattctaa

SEQ ID NO: 58 - protein, 412 aa, Scheffersomyces stipites
MVSVLSFTRQVFLTLALSLLVNDSVALDKRSPGFLKLDFTVARGQGKESLNLTSTNKPYFVYHEKSKRATGEPEVTLINEKTFYATDLEIGSNKQAVTTLIDTGSSDLWVVDKSAKCQVTETGQSSTYCYEYGVYDHTKSSSYHSLGSKFSIEYVDGTSSTGTWVKDDVYFAGTTTGVTQLQFGDVTTTSSGFGVLGIGYEANESTNTEYPNLPVLLKTQGVIAKTAYSLYLAQEDATSGSVIFGGIDQAKYSGSLITLPVTSSRELAIHLGSVAINGKTISANLNPVLDSGTTLTYLPTKIVASIASALSGTADSSVGYIINCNQPSNKYLTFNFDSGATINVPLSELAIDLYLTNGQKYQYCALGVFATSSTPILGDNFLRSAYVAYDLDDNTISLAQVKYTTAENIVPF

SEQ ID NO: 59 - DNA, 1209 bp, Lodderomyces elongisporus
  source 1..1209: mol_type = genomic DNA
atggtcagtgttttcaacttgaccaagcaagctttgactactttggcttttgctttgttggctcaaggtatcgttattccagaagatttgggtaaaagatctggtccaggtttcatctctttggatttcgatgttattagaccaccagtcatcgttaactctactgattctaatgctgctttgtctgatgctgccttgagaaagagaaagaccatttccttgtccttgattgatgaaggtccatcttacgcttccaagattactattggttccaacaagcaacaacaaaccgttgttattgataccggttcttctgatttgtgggttgttgattcaaacgctcaatgccaagataacgttcaatgtaagaacgacggtacttacaacccatcttcttctactacctacaaaaacttgaacaccccattcgctattagatacggtgatggttctacttctcaaggtacttggggtttggaaactgttggttttggtggtatttctatcaccggtcaacaattcgctgatgttactactacctctgttaatcaaggtattttgggtatcggttacaagaccaatgaagctaatgctaactacgataacgtcccagttactttgaagaagcaaggtattatttctaccaacgcctactccttgtatttgaatgctccaaatgctgcatccggtactattatctttggtggtgttgataacgctaagtactctggttcattgatcaaagaacaagtcacccaatccaatcaattgaccatttctttgggttccattaactacgctggtacaacttactctaacaacaatggtgatgccttgttggattctggtactactttgacttatttgaccccagatgttgcttctgctattgctgaacaagctggtgctcattatgttacttatccagatggttctggtttatgggaaattggttgtgatgcttctacatccggtaacgttgtttactcttttgctaacggtgctaagattaccgttccattgtctgaattggtttacggttcttcaggtgacggttattgtgtttggggtattcaacaagaagaagatttcgcaatcttgggtgacaactttttgagacatgcttacttgttgtacaatttggatgccaacaccgtttctattgcccaagttaagtataccaccacctcttctattgctgctgtttaa

SEQ ID NO: 60 - protein, 402 aa, Lodderomyces elongisporus
MVSVFNLTKQALTTLAFALLAQGIVIPEDLGKRSGPGFISLDFDVIRPPVIVNSTDSNAALSDAALRKRKTISLSLIDEGPSYASKITIGSNKQQQTVVIDTGSSDLWVVDSNAQCQDNVQCKNDGTYNPSSSTTYKNLNTPFAIRYGDGSTSQGTWGLETVGFGGISITGQQFADVTTTSVNQGILGIGYKTNEANANYDNVPVTLKKQGIISTNAYSLYLNAPNAASGTIIFGGVDNAKYSGSLIKEQVTQSNQLTISLGSINYAGTTYSNNNGDALLDSGTTLTYLTPDVASAIAEQAGAHYVTYPDGSGLWEIGCDASTSGNVVYSFANGAKITVPLSELVYGSSGDGYCVWGIQQEEDFAILGDNFLRHAYLLYNLDANTVSIAQVKYTTTSSIAAV

SEQ ID NO: 61 - DNA, 1197 bp, Candida albicans
  source 1..1197: mol_type = genomic DNA
atgtttttgaagaacatcttcattgccttggctatcgctttgttggttgatgctactccaactactacaaaaagatctgctggtttcgttgccttggatttctctgttgttaagactcctaaagctttcccagttaccaatggtcaagaaggtaaaacctctaagagacaagctgttccagttacattgcacaacgaacaagttacttacgctgctgatattaccgtcggttctaacaatcaaaagttgaacgttatcgtcgacaccggttcttcagatttgtgggttccagatgttaacgttgattgtcaagttacctactctgatcaaaccgctgatttctgtaaacaaaagggtacttatgacccatctggttcatctgcttctcaagatttgaataccccattcaagattggttacggtgatggttcttcatctcaaggtacattatacaaggataccgttggttttggtggtgtctccattaagaatcaagttttggctgatgttgactccacctctattgatcaaggtattttgggtgttggttacaagactaatgaagctggtggttcctatgataatgttccagtcactttgaaaaagcaaggtgttattgctaagaacgcctactccttgtatttgaattctccagatgctgctaccggtcaaattatcttcggtggtgttgataatgctaagtactccggttctttgattgctttgcctgttacttctgacagagaattgagaatctctttgggttccgttgaagtttctggtaagactattaacaccgataacgtcgatgttttggttgattctggtactaccattacctacttgcaacaagatttggccgatcaaatcattaaggccttcaacggtaaattgacccaagattctaatggtaactccttctacgaagttgactgtaacttgtctggtgatgtcgttttcaacttctccaagaacgctaagatttctgttccagcttctgaatttgctgcttcattgcaaggtgatgatggtcaaccatatgataagtgccaattgttgttcgatgtcaacgatgctaacatcttgggtgacaattttttgagatccgcctacatcgtttacgatttggatgataacgaaatttccttggcccaagtcaagtatacctctgcttcttctatttctgctttgacttaa

SEQ ID NO: 62 - protein, 398 aa, Candida albicans
MFLKNIFIALAIALLVDATPTTTKRSAGFVALDFSVVKTPKAFPVTNGQEGKTSKRQAVPVTLHNEQVTYAADITVGSNNQKLNVIVDTGSSDLWVPDVNVDCQVTYSDQTADFCKQKGTYDPSGSSASQDLNTPFKIGYGDGSSSQGTLYKDTVGFGGVSIKNQVLADVDSTSIDQGILGVGYKTNEAGGSYDNVPVTLKKQGVIAKNAYSLYLNSPDAATGQIIFGGVDNAKYSGSLIALPVTSDRELRISLGSVEVSGKTINTDNVDVLVDSGTTITYLQQDLADQIIKAFNGKLTQDSNGNSFYEVDCNLSGDVVFNFSKNAKISVPASEFAASLQGDDGQPYDKCQLLFDVNDANILGDNFLRSAYIVYDLDDNEISLAQVKYTSASSISALT

SEQ ID NO: 63 - DNA, 1197 bp, Candida albicans
  misc_feature 1..1197: note = SC5314
  source 1..1197: mol_type = genomic DNA
atgtttttgaagaacatcttcattgccttggctattgctttgttggctgatgctactccaactacttttaacaattctccaggtttcgttgccttgaacttcgatgttattaagacccataagaacgttactggtccacaaggtgaaatcaacactaatgttaacgtcaagagacaaaccgttccagtcaagttgattaacgaacaagtttcctacgcctccgatattactgttggttctaacaagcaaaagttgaccgttgttattgacaccggttcttcagatttgtgggttccagattctcaagtttcttgtcaagctggtcaaggtcaagatccaaatttctgtaagaacgaaggtacttactccccatcttcttcatcttcatctcaaaacttgaactccccattctccattgaatatggtgatggtactacttctcaaggtacttggtacaaagataccattggtttcggtggtatttccatcactaagcaacaattcgctgatgttacttccacctctgttgatcaaggtattttgggtattggttacaagacccatgaagctgaaggtaactatgataacgttccagttaccttgaagaatcaaggtatcatttccaagaacgcctactccttgtacttgaattctagacaagctacctccggtcaaattatctttggtggtgttgataacgctaagtactccggtactttgattgctttgccagttacttctgacaacgaattgagaatccatttgaacaccgttaaggttgctggtcaatctatcaatgctgatgttgatgttttgttggactctggtactaccattacctacttgcaacaaggtgttgccgatcaagttatttctgcttttaacggtcaagaaacctacgatgctaatggtaacttgttctacttggttgactgtaacttgtccggttctgttgattttgctttcgataagaacgccaagatttctgttccagcttctgaattcactgctccattatacactgaagatggtcaagtttacgaccaatgccaattgttgtttggtacatccgattacaacatcttgggtgacaatttcttgagatccgcttacatcgtttacgatttggacgataacgaaatctctttggcccaagttaagtacactaccgcttctaatattgctgctttgacctaa

SEQ ID NO: 64 - protein, 398 aa, Candida albicans
  REGION 1..398: note = SC5314
MFLKNIFIALAIALLADATPTTFNNSPGFVALNFDVIKTHKNVTGPQGEINTNVNVKRQTVPVKLINEQVSYASDITVGSNKQKLTVVIDTGSSDLWVPDSQVSCQAGQGQDPNFCKNEGTYSPSSSSSSQNLNSPFSIEYGDGTTSQGTWYKDTIGFGGISITKQQFADVTSTSVDQGILGIGYKTHEAEGNYDNVPVTLKNQGIISKNAYSLYLNSRQATSGQIIFGGVDNAKYSGTLIALPVTSDNELRIHLNTVKVAGQSINADVDVLLDSGTTITYLQQGVADQVISAFNGQETYDANGNLFYLVDCNLSGSVDFAFDKNAKISVPASEFTAPLYTEDGQVYDQCQLLFGTSDYNILGDNFLRSAYIVYDLDDNEISLAQVKYTTASNIAALT

SEQ ID NO: 65 - DNA, 1221 bp, Candida dubliniensis
  misc_feature 1..1221: note = CD36
  source 1..1221: mol_type = genomic DNA
atgtttttgaagaacatcttcatcaccttggccattgctttgttggttgatgctattccaactacctctaagtctaagaattctccaggtttcgttgccttgaacttcgatgttattaagacccataagaacgtcactggtcaacaaggtaatggtaaggttactcataaggttaatgctgccaacgttaagagacaaactgttccagttactttgttgaacgaacaagtttcctacgcctctgatattactgtcggttctaacaatcaaaagttgaccgttgttatcgacaccggttcttcagatttgtgggttccagatactgatgtttcttgtcaaacttcctacgaaggtcaagatccaaacttctgtaaagactacggtacttactctccatcctcttcatcatcttctcaagatttgaacaacccattctccatcgaatatggtgatggtactacttctcaaggtacttggtacaaagataccattggtttcggtggtatttccatcactaagcaagaattcgctgatgttacttccacctctgttgatcaaggtattttgggtattggttaccaatctcatgaagccgaaggttactatgataatgttcctgttaccttgaagaagcaaggtattattgctaagaacgcctactccttgtacttgaactctagacaatctgcttccggtcaaattatctttggtggtgttgataacgctaagtactccggttctttgattactttgccaaccacttctaactccgaattgagaattcatttgaacaccgttactgttgccggtcaatctatcaatgctgatgttgttgttttgttggactccggtactaccatttcttacttgcaacaaggtgttgccgatcaagttatttctgcttttaacggtcaagaaacctacgatgctaatggtaacttgttctacttggttgactgtaacttgtctggttctgttgaattcgctttcgataagaacgctaagatttctgttccagcttctgaattcactgccccattatattcatctgatggtcaagtttacgaccaatgccaattgttgtttggtacttccgattacaacatcttgggtgacaatttcttgagatccgcttacatcgtttacgatttggatgacaacgaaatctctttggcccaagttaagtataccaccgcttctaatattgctgctttgacctaa

SEQ ID NO: 66 - protein, 406 aa, Candida dubliniensis
  REGION 1..406: note = CD36
MFLKNIFITLAIALLVDAIPTTSKSKNSPGFVALNFDVIKTHKNVTGQQGNGKVTHKVNAANVKRQTVPVTLLNEQVSYASDITVGSNNQKLTVVIDTGSSDLWVPDTDVSCQTSYEGQDPNFCKDYGTYSPSSSSSSQDLNNPFSIEYGDGTTSQGTWYKDTIGFGGISITKQEFADVTSTSVDQGILGIGYQSHEAEGYYDNVPVTLKKQGIIAKNAYSLYLNSRQSASGQIIFGGVDNAKYSGSLITLPTTSNSELRIHLNTVTVAGQSINADVVVLLDSGTTISYLQQGVADQVISAFNGQETYDANGNLFYLVDCNLSGSVEFAFDKNAKISVPASEFTAPLYSSDGQVYDQCQLLFGTSDYNILGDNFLRSAYIVYDLDDNEISLAQVKYTTASNIAALT

SEQ ID NO: 67 - DNA, 1356 bp, Neurospora tetrasperma
  source 1..1356: mol_type = genomic DNA
gacttggacggtatcccatcttttaacaatggttccttgagacattctaccaacgctagatgtagatgtccaaaagatggtggttactacgttaacgttactattggtactccaggtagaaacttgtccttgcatttggatactggttcttcagatacttgggttaactccccatcttctattttgtgccaaaacgaagataagccatgcgaatattctggtacttacttggctaacgattcctctacctacgagtatatctccaaccacttcgatatcaagtacgttgatggttctggtgctagaggtgattatgcttctgatactttcactatcggtaacaccaagttgaacagattgcaattcggtatcggttactcttctactaatgctcaaggtttgttgggtattggttacgctttgtctgaagttcaaactagagctggtttgccagcttataacaatttgccagctcaaatggttgctgacggtttgattaactctaacgcttactctatctggttgaacgatttggatgctccaactggtactattttgtttggtggtgttgatgctgctaagtacgaaggtgatttgttgactttgccagttcaaactccagaaaagggtacttacaagaacttcatggttactatgaccggtatgtccttgtctcaatctcaatcttcttcatccgataagggtaacggtgataacactacccaaatctctaaggataatttggctttggccgttttgttggatacaggttctactttgtcttacttgccatccgaattgatcaagccattatacgatgccattggtatcgagtatatcaccgatccagatggtaaagttgatggttatgctccatgtcacttgatgtcatcttctcaatctgtcatgttctcattctccagtccattgcaaattgctgttccaatgaacgaattgatcatcaacagaaccttccacggtaaattgccaagaatgccagatggtgttactgatgcttgtattttcggtatccaagaaagaaatggtactggtgctaatactttgggtgatacctttttgagatccgcctacgttgttttcgatttggataacaacgaaatctccatggctcaaactagattcaatgctactgctaccgacttgaaagaaatcaaaaaaggtaaaggtggtgttccaggtgctaaggctgttgaaaatccagttgaagctacttctggtttgactggttctgaaggtggtatctatgttaatggtgctgctggtgaattgaatgttggtatgggtatggcttggggtttgttggtttctggtgctatggtttttgttggtttgtaa

SEQ ID NO: 68 - protein, 452 aa, Neurospora tetrasperma
MDLDGIPSFNNGSLRHSTNARCRCPKDGGYYVNVTIGTPGRNLSLHLDTGSSDTWVNSPSSILCQNEDKPCEYSGTYLANDSSTYEYISNHFDIKYVDGSGARGDYASDTFTIGNTKLNRLQFGIGYSSTNAQGLLGIGYALSEVQTRAGLPAYNNLPAQMVADGLINSNAYSIWLNDLDAPTGTILFGGVDAAKYEGDLLTLPVQTPEKGTYKNFMVTMTGMSLSQSQSSSSDKGNGDNTTQISKDNLALAVLLDTGSTLSYLPSELIKPLYDAIGIEYITDPDGKVDGYAPCHLMSSSQSVMFSFSSPLQIAVPMNELIINRTFHGKLPRMPDGVTDACIFGIQERNGTGANTLGDTFLRSAYVVFDLDNNEISMAQTRFNATATDLKEIKKGKGGVPGAKAVENPVEATSGLTGSEGGIYVNGAAGELNVGMGMAWGLLVSGAMVFVGL

SEQ ID NO: 69 - DNA, 1443 bp, Podospora anserine
  source 1..1443: mol_type = genomic DNA
atgaagaccgccacctccttgttggttgttgctgcttctttggctggtcaaactattgatgctttgtctttgccatctactactccaactcaacaacaacaaagaagagatggttctggtccaagagttgttggtatggatattcaaagaagaaccccaaagaacccattgcatagagatcatttgagaaagagaggttccgttgaagttgacttggataatcaagaaaccttgtacttcatcaacggtactattggtactccaccaaagtctttgagattgcatttggatactggttcctctgatttgtgggttaatactccatcttcatccttgtgtactcaatcttctgctccatgtaaatacgctggtacttattctgctaacggttcttctacctacgagtatattggttcctggttcaacatctcctacgttgatggttcaggtgcttctggtgattatgtttctgatactgttactttcggtgatgccactttggatagattgcaatttggtatcggttactcctctaacaacgctcaaggtattttgggtattggttacccaatcaacgaagttcaagttggtagagctggtatgagaccatacaacaatttgccagctcaaatggttgctgatggtttgattcaaactaacgcttactctttgtggttgaacgatttggatgctgataccggtaacattttgtttggtggtgttgataccgaaaagttcgttccaccattgatgtctttgccagttgaatctgaagctggtgtttatgccgaattcatgattactttgaccaaggtcgaattgggttctgctcaagttggtggtgatttggctttggctgttttgttagatactggttcttccttgacttacttgccagatagaatggttcaagacatcttcgatttggttgatgctcaatatgatcctgaagctaatgctgcttatgttccatgttctttggctgataacgaaaccgctgttttgtctttcactttcactgaacctaccatcaatgtcggtatggatgaattggttttggatttggttacctcctctggtagaagaccagttttttctgatggtactgaagcttgcttgtttggtattgctccagctggtgaaggtactaatgttttgggtgatactttcttgagatccgcctatgttgtttacgacttggaaaacaacgaaatttctttggctgctaccagattcaactctactggtactagagttgaagaaatcggtaaaggtgaaggtggtgttccaggtgctacaaaagttgaaaatccaactaaggctaccgaaggtttggatggtccaaatggtttgggtggtatttctgctggtaacaaaagaggtttggaagttggagttgtttggttggttgctggtatggttggtgttttattggttgtttaa

SEQ ID NO: 70 - protein, 480 aa, Podospora anserine
MKTATSLLVVAASLAGQTIDALSLPSTTPTQQQQRRDGSGPRVVGMDIQRRTPKNPLHRDHLRKRGSVEVDLDNQETLYFINGTIGTPPKSLRLHLDTGSSDLWVNTPSSSLCTQSSAPCKYAGTYSANGSSTYEYIGSWFNISYVDGSGASGDYVSDTVTFGDATLDRLQFGIGYSSNNAQGILGIGYPINEVQVGRAGMRPYNNLPAQMVADGLIQTNAYSLWLNDLDADTGNILFGGVDTEKFVPPLMSLPVESEAGVYAEFMITLTKVELGSAQVGGDLALAVLLDTGSSLTYLPDRMVQDIFDLVDAQYDPEANAAYVPCSLADNETAVLSFTFTEPTINVGMDELVLDLVTSSGRRPVFSDGTEACLFGIAPAGEGTNVLGDTFLRSAYVVYDLENNEISLAATRFNSTGTRVEEIGKGEGGVPGATKVENPTKATEGLDGPNGLGGISAGNKRGLEVGVVWLVAGMVGVLLVV

SEQ ID NO: 71 - DNA, 1425 bp, Grossmannia clavigera
  source 1..1425: mol_type = genomic DNA
atgaaggctgctgctttggctattgctgcttctatttgggttaagttggctttgtctttgtccttgcaacatagaagagatggtactgctgctagagttgttggtatgactactgaaagaagacatgttgccaacccaatccaaagagatagattgagaagaagaggtgctgttgctgctggtttagctaatgaagaaaccttgtacttcattaacgctaccttgggtactccaccacaatctttgagattgcatattgataccggttcctctgatttgtgggttaatactccatcttctaccttgtgccaatctagaacaaaaccatgttcttacgctggtacttataccgctaattcttcttctacctacgaatacgttggttcctacttcaacatctcttacgttgatggttctggtgcttctggtgattatgttgctgataccatttctttcggtgataacgctactttggctagattgcaatttggtattggttacgcctcttcatcttctcaaggtgttttgggtattggttatgctgctaacgaagttcaagttggtagagctggtagacaagcttataacaatttgccagctcaattgatggccgatggtcaaattgcttctaatgcttattctttgtggttgaacgatttggatgctaacaccggttctattttgtttggtggtgttgatactggtcaatacgttggtcaattgcaaactttgccagttcaaaaacaagccggtgatttctctgaattcttggttactttgaccggtttacaattgggttctgctactttagctgatgatttggctttggctgttttgttggattctggttcttctttgacttacttgccagataacactgtcgaaaccttgtataatagagttggtgctacctacgattcttctgaaggtgctgcttatgttgaatgttcattggctagatctaacaccagtttgacctttagtttctccggtttacacattgctgttggtatggatgaattggtcttggatttgttcactacctctggtaaaagaccacaattccaaaatggtcaagatgcttgtttgtttggtgttgctccagctggttcttctactaatgttttgggtgataccttcttgagatccgcttatgttgtttacgacttgtccaacaatcaaatctctttggctcaaacctctttcaacgctacttctacaaacgttttggaaattgctgctggtactactggtgttccagatgctactgctgttgctaatccagtttctgctactcaaggtttggttatcttgggtaattctaacactgctaagtctgctgctgttccatcttttccaggtactccagctgctttagctgtttttgttgttgctgcttctgttatggctggttggtctttgtaa

SEQ ID NO: 72 - protein, 474 aa, Grossmannia clavigera
MKAAALAIAASIWVKLALSLSLQHRRDGTAARVVGMTTERRHVANPIQRDRLRRRGAVAAGLANEETLYFINATLGTPPQSLRLHIDTGSSDLWVNTPSSTLCQSRTKPCSYAGTYTANSSSTYEYVGSYFNISYVDGSGASGDYVADTISFGDNATLARLQFGIGYASSSSQGVLGIGYAANEVQVGRAGRQAYNNLPAQLMADGQIASNAYSLWLNDLDANTGSILFGGVDTGQYVGQLQTLPVQKQAGDFSEFLVTLTGLQLGSATLADDLALAVLLDSGSSLTYLPDNTVETLYNRVGATYDSSEGAAYVECSLARSNTSLTFSFSGLHIAVGMDELVLDLFTTSGKRPQFQNGQDACLFGVAPAGSSTNVLGDTFLRSAYVVYDLSNNQISLAQTSFNATSTNVLEIAAGTTGVPDATAVANPVSATQGLVILGNSNTAKSAAVPSFPGTPAALAVFVVAASVMAGWSL

SEQ ID NO: 73 - DNA, 1431 bp, Chaetomium thermophilum
  source 1..1431: mol_type = genomic DNA
atgaagtccacctctttcttcgccgttttgtcatcctttttcttcaaacctattccagccgtttctttgccacaaactagagaagatggtttgagagttgttaacttggaaaccgaaagaagaccagctagacatccagttcatagagataacttgagaaagagaggtactgttaccgttggtttggataatgaagaaaccttgtacttgatcaacatcaccattggtactccaccaacatctttgagattgcatattgataccggttcctctgatttgtgggttaatactccaggttcttctttgtgccaatcttctgataatccatgtgcttttgctggtacttactctgctaattcttcttccacttacgaatacgttggttcctggttcaacatctcttatgttgatggttctggtgcttctggtgattatgcttctgataccattactatcggtggtaagaagatcgaaagattgcaattcggtatcggttacgaatcctctaacaatcaaggtattttgggtattggttacccattgaacgaagttcaagttggtagagctggtaaaaaggcttacaacaatttgccagctcaattggttgctgatggtgctattagatctaaggcttattctttgtggttgaacgatttgtctgctaacaccggtaacattttgttcggtggtattgataccgaaagatactctggtactttgaagatcttgccagttgaatctgaagctggtatctacgctgaattcttcattactttgaccaagttgcaattgggtgatcatgctattggtggtgatttggctttggctgttttgttggatactggttcttcattgacttacttgccagatccattggtccaagaaatctattctttggttggtgctacttttgatgctggtgctaatgctgcttatattccatgttctgctgctcaaaacactacccagttgttgttcactttcaccgaacctactattgccgttgatatgaacgaattggttttggacatcttggtttccaacggtaaaagaccaactttctctaatggtcaaccagcttgtttgtttggtattgctccagctggtgctggtactaatgttttgggtgatacttttttgagatccgcctacgttgtttacgatttggataacaacgaaattgctttggctccaactagattcaacgctactacttctagagttttggaaatcggtactggtgaaaatgctattccaggtgctactagagttgctaatccagttgctgctactgaaggtttacatggtccaaatgctgctactggtattaacaatgctgctgctggttctattatcttcacttctggtttgactgcttctggtttagctgctggtgttgctatagttgttggtatgttgttgatgatttaa

SEQ ID NO: 74 - protein, 476 aa, Chaetomium thermophilum
MKSTSFFAVLSSFFFKPIPAVSLPQTREDGLRVVNLETERRPARHPVHRDNLRKRGTVTVGLDNEETLYLINITIGTPPTSLRLHIDTGSSDLWVNTPGSSLCQSSDNPCAFAGTYSANSSSTYEYVGSWFNISYVDGSGASGDYASDTITIGGKKIERLQFGIGYESSNNQGILGIGYPLNEVQVGRAGKKAYNNLPAQLVADGAIRSKAYSLWLNDLSANTGNILFGGIDTERYSGTLKILPVESEAGIYAEFFITLTKLQLGDHAIGGDLALAVLLDTGSSLTYLPDPLVQEIYSLVGATFDAGANAAYIPCSAAQNTTQLLFTFTEPTIAVDMNELVLDILVSNGKRPTFSNGQPACLFGIAPAGAGTNVLGDTFLRSAYVVYDLDNNEIALAPTRFNATTSRVLEIGTGENAIPGATRVANPVAATEGLHGPNAATGINNAAAGSIIFTSGLTASGLAAGVAIVVGMLLMI

SEQ ID NO: 75 - DNA, 1428 bp, Myceliophthora thermophila
  misc_feature 1..1428: note = ATCC 42464
  source 1..1428: mol_type = genomic DNA
atgagatcctcctccttgttggttgctttggctactttgattcaaatcactcatggtttggctttgccagaaagaagacatggtccatctgttttgggtttggaaatgcaaagaagagcaccaagaaatccattgcacagagataagcgtagaagaaagagaggtactttggaagttggtttggataacgaagaaaccttgtacttcatcaacggtactgttggtactccaccaaaaccagttagattgcatattgataccggttcctctgatttgtgggttaatactccagcttctgaattgtgtgcttctgctaatgatccatgtgcttttgctggtacttactctgctaatagttcttccacctaccagtatatctcttccaacttcaacatctcctacgttgatggttctggtgcttctggtgattatgtttctgatactgttaccatcggttcccaaaagatcgatagattgcaatttggtgtcggttactcttctaccaacgatcaaggtattttgggtattggttacccattgaacgaagttcaagttggtagagctggtttaagaccatacaacaatttgccagctcaattggttgctgatggtgttattagatctatggcctactctatctggttgaacgatttggatgctaacaccggtaacattttgtttggtggtgttgatactgaaaagtacgctccaccattattgtctttgccagttgaatctgcttctggtgttttctccgaattcatgattactttgaccggtttgaagttgggttctcaaactattggtccatccgatttggctattgctgttttgttggatactggttcttctttgacttacttgccagatgctttggtttctgatatctatgctgctgttggtgctgtttttgatggtgatgctaatgctgcttatgttccatgttctttggctagagatgcttctgctccaccattgacttttactttttctgaaccagctattgccgttggtatggatgaattggttttggatttggttactgcctctggtagaagaccaacttttgataatggtactccagcttgcttgtttggtattggtccagctggtgcaggtacttatgttttgggtgatacttttttgagatccgcctacgttgtttacgatttggataacaacgaaattgctttggctccaaccagattcaattcttctgctactagagttgtcgaaatcggtactggtcaagatgctgttccaggtgctacaagagtttctaatccagttaaggctactgaaggtttgagaggtatgaatgctaaaaagaatgctgctgctgctgttgctggtttgggttctggtggtatgagattgatggttgcttgtgctacattggttgttgttgttgtaggtggtttggtttaa

SEQ ID NO: 76 - protein, 475 aa, Myceliophthora thermophila
  REGION 1..475: note = ATCC 42464
MRSSSLLVALATLIQITHGLALPERRHGPSVLGLEMQRRAPRNPLHRDKRRRKRGTLEVGLDNEETLYFINGTVGTPPKPVRLHIDTGSSDLWVNTPASELCASANDPCAFAGTYSANSSSTYQYISSNFNISYVDGSGASGDYVSDTVTIGSQKIDRLQFGVGYSSTNDQGILGIGYPLNEVQVGRAGLRPYNNLPAQLVADGVIRSMAYSIWLNDLDANTGNILFGGVDTEKYAPPLLSLPVESASGVFSEFMITLTGLKLGSQTIGPSDLAIAVLLDTGSSLTYLPDALVSDIYAAVGAVFDGDANAAYVPCSLARDASAPPLTFTFSEPAIAVGMDELVLDLVTASGRRPTFDNGTPACLFGIGPAGAGTYVLGDTFLRSAYVVYDLDNNEIALAPTRFNSSATRVVEIGTGQDAVPGATRVSNPVKATEGLRGMNAKKNAAAAVAGLGSGGMRLMVACATLVVVVVGGLV

SEQ ID NO: 77 - DNA, 1425 bp, Magnaporthe oryzae
  misc_feature 1..1425: note = 70-15
  source 1..1425: mol_type = genomic DNA
atgagattgatcgcctccttggctttggctgcttctttggcttcttctattactaatggttccactattccaagaagagcttctggtactccagctgctccaagagttattggtttggaaactgaaagacaacacatcccaaatccattggaaagagacagattgagaagaagagctgctgttatggctactttggataatgaacaaaccttgtacttcgtcaacgtcagtattggtactccaccacaaaaattgagattgcatttggataccggttcctctgatttgtgggttaatactccagattctaagttgtgctccgtttcttcacaaccatgtagatttgctggtactttctctgctaactcatcttctacctaccagtatatcaactccgttttcaacatctcctacgttgatggttctggtgctaatggtgattacgtttctgatatggttactgtcggtaacaccaagatcgatagattgcaatttggtatcggttacacctcttcatctgctcaaggtattttgggtgttggttacgaagctaacgaagttcaagttggtagagcacaattgaagccatacagaaatttgccatccagaatggttgaagaaggtttgattgcttctaacgcctactccttgtatttgaacgacttgcaatctaacaagggttccattttgttcggtggtattgatactgaacagtacaccggtacattgcaaaccgttccaattcaacctaatggtggtagaatggccgaattcttgattactttgacctctgtttctttgacctccgcttctattggtggtgataagttggctttagctgttttgttggattccggttcttctttgacttacttgccagatgatatcgtcaagaacatgtattctgctgttggtgctcaatacgattctaatgaaggtgctgcttatgttccatgttctttggctagagatcaagctaactctttgacgttttccttcagtggtattccaatcgttgttccaatgaacgaattggttttggatttggttacctccaatggtagaagaccatcttttagaaatggtgttccagcttgtttgtttggtgttgctccagctggtaaaggtactaatgttttgggtgataccttcttgagatccgcttatgttgtttacgacttggaaaacaacgctatctctttggctcaaacttctttcaacgctactaagtccaacgtcaaagaaattggtaagggttctaatccagttccaggtgctgttgctgtttctcaaccagttgctgctacttctggtttgtctcaaaatggtggtaatagatctggttcaggtgctattgctagagctgttccaactttgttgttggttggtggtattttctctggttctttgttgactttgttttaa

SEQ ID NO: 78 - protein, 474 aa, Magnaporthe oryzae
  REGION 1..474: note = 70-15
MRLIASLALAASLASSITNGSTIPRRASGTPAAPRVIGLETERQHIPNPLERDRLRRRAAVMATLDNEQTLYFVNVSIGTPPQKLRLHLDTGSSDLWVNTPDSKLCSVSSQPCRFAGTFSANSSSTYQYINSVFNISYVDGSGANGDYVSDMVTVGNTKIDRLQFGIGYTSSSAQGILGVGYEANEVQVGRAQLKPYRNLPSRMVEEGLIASNAYSLYLNDLQSNKGSILFGGIDTEQYTGTLQTVPIQPNGGRMAEFLITLTSVSLTSASIGGDKLALAVLLDSGSSLTYLPDDIVKNMYSAVGAQYDSNEGAAYVPCSLARDQANSLTFSFSGIPIVVPMNELVLDLVTSNGRRPSFRNGVPACLFGVAPAGKGTNVLGDTFLRSAYVVYDLENNAISLAQTSFNATKSNVKEIGKGSNPVPGAVAVSQPVAATSGLSQNGGNRSGSGAIARAVPTLLLVGGIFSGSLLTLF

SEQ ID NO: 79 - DNA, 1770 bp, Kluveromyces lactis
  source 1..1770: mol_type = genomic DNA
atgaagttgtccgacgtttgcttgggtgctttgttggcttctttgggttctgctggttatattaccaagagagatgtttcccaagttggtgaagctaacaaaaagcacgttttcatgtccttcgataagttgagaggtaacgatgcttctgaagcttctttgtctaaaagaagagttggtcacttgaagaaaagagctgacggttacgttgatgtcgaaatcgataatcaaaacaccttctactccgtcgatttggaaattggtactccagctcaaaaagttggtgttttgatagataccggttcctctgatttgtgggttccaggttctggtaatccattttgttctgcttctagatcctcctcaaagaagaagagacaagattataccgccttgttgtcctctttgttgtctgtttttggtactgatgttgctaccgattacacctatactggtattgcttctgctactggttcagctggtactcaaactgttccaactgctttgacttctggtactgttgctgctacttctattagatctgttccatcttctttggctaccttggattgctctgaatttggtactttcgatacctccaaatcctcttcttggcaatctaatgataccagattctacatctcttacgccgatggtacttttgctgatggtgattggggtgttgacgaaattcatttggatgatgttaacgtcaccggtttgtcttttgctgtttctaactacaccgattcccaatttggtgttttgggtattggtttgactggttctgaatctacttactccggtcaattgtctacctacaacagataccaatacgacaacttcccaatcgtcttgcaaagaaacggtgttattgaaaagaacgcctactccgttttcttgaacgatttggatgctgattccggttctattttgtttggtgctgttgatcattctaagtacaccggtacattatacaccgttccaatggttaatgccttgagatctattggttacaccgaacaaatcagattgtccatcacattgcaaggtattggtgttacttcttcctacggtaacgaaactgttacccaaactaagtatccagctttgttggatactggtgctactttttgttacttcccatcttctttagctgctgctattgcttcttctgtttctgctacttactcttcctcttctggttactacttcgttgattgtgattctggttccgattacgatttggtttttgattttggtggtttccacatcatctccccattgtctgattatatcgttaccacctcttcttcatcccaatgtgttttaggtatcttgccacaatccgataacgaaattactttgggtgatgctttcttgacctctgcttatgttgtttacgacttggaagaattggaaatctctttggctcaagcttcttatacttctggtgacgaaaagatcgaagtcatcagagattctgttccatcagctgttgctgctccaggttattcttctacttggtctactgctgcttctttatctactggtggtaacattttcaccgttaccaacaatgctaacggtactgtttctactggtactggttcttctggttcaggttcatctacatctggtaattctggttcaacttctaccggttccagttctaaaaaagaaggtgctgcttcttcattgccagttccacatttggctgctttgatttgtttgttgttgtccgctgtttccatcccatctttgttttaa

SEQ ID NO: 80 - protein, 589 aa, Kluveromyces lactis
MKLSDVCLGALLASLGSAGYITKRDVSQVGEANKKHVFMSFDKLRGNDASEASLSKRRVGHLKKRADGYVDVEIDNQNTFYSVDLEIGTPAQKVGVLIDTGSSDLWVPGSGNPFCSASRSSSKKKRQDYTALLSSLLSVFGTDVATDYTYTGIASATGSAGTQTVPTALTSGTVAATSIRSVPSSLATLDCSEFGTFDTSKSSSWQSNDTRFYISYADGTFADGDWGVDEIHLDDVNVTGLSFAVSNYTDSQFGVLGIGLTGSESTYSGQLSTYNRYQYDNFPIVLQRNGVIEKNAYSVFLNDLDADSGSILFGAVDHSKYTGTLYTVPMVNALRSIGYTEQIRLSITLQGIGVTSSYGNETVTQTKYPALLDTGATFCYFPSSLAAAIASSVSATYSSSSGYYFVDCDSGSDYDLVFDFGGFHIISPLSDYIVTTSSSSQCVLGILPQSDNEITLGDAFLTSAYVVYDLEELEISLAQASYTSGDEKIEVIRDSVPSAVAAPGYSSTWSTAASLSTGGNIFTVTNNANGTVSTGTGSSGSGSSTSGNSGSTSTGSSSKKEGAASSLPVPHLAALICLLLSAVSIPSLF

SEQ ID NO: 81 - DNA, 1500 bp, Ashbya gossypii
  misc_feature 1..1500: note = ATCC 10895
  source 1..1500: mol_type = genomic DNA
atgatcgctcaaattgctgctttgggtgctgttttggctactaatgttgcttgtgcttctttggtcgaaagagaagaagctgctcaattcgttagaatggatttcgataagaagagaggtccatctttttcagaagcttggtctggtagagctgctgttccaagattggctaaaagaaatgattgggtcgatatcgaaatcgataatcaacaaatgttctactccgtcaacttgtccattggtactccaccacaagaagttagagttttgatggatactggttcctctgatttgtgggttgttggtgctggtgttagatgtggtccaaacaattatccaaacccaaatcaattgaactgctacgaacatggttctttcgatacctctagatcttctacttggaaggataacaacacccaattccatatcagatacggtgattctacttacgctcatggtacttggggtactgatagattggatttgggtcaagctaatgttgatggtttgacttttgctgttgctcatgcttctaattcctctgttgctgttttgggtattggtttgccagctatggaaactactatgtcctctcaacaattcggttaccaatacgataacttgccaatggtcttgaagagaaacaacgtcatcaaaaagaccgtctactccatgttcatcaacgaattgaatgctaagaccggttctgttttgtttggtgctgttgatcattctaagtacaagggtactttgaacaccgttccattggttaatgctaactacagaagaagagttaacaagccagttgaattgcatgttaccttggctgctattggtttacacactggtagagatgaagataaggttcaaactttgttgggttctaaggttcctgctttgttggattctggtactactttgacttacttcccaatgcaattggcagatagattggctagagctgctggtgctagatggcattctgaagaagttggttatatcgttgactgcaactccagaagattgcatcaaactgcttacatctacgatttcggtggtttccaaattagatccccattgtctgattactccatgatgactaatgttagaggtacttgcagattcggtatcatgccacattcttccgattacgttattttgggtgatgttttcttgaccagagcctacgttgtttttgatttggaagctttggaagtctctatggctcaagctaattatgaaggtggtaaagaacaaatcgaagttattgctggtgctgttcctagagctgttagagcaccaggttataatgatccatggaaagcttataccccattggtttttaatggtgaattcggtaacttgactgctaccgcttctgattcttcttctggttctgttggttctagatctaactctgctttgactttgactccaccatctttgtctgttatgttgtttgctgcatctttgttgggtgctgctactagaattatctaa

SEQ ID NO: 82 - protein, 499 aa, Ashbya gossypii
  REGION 1..499: note = ATCC 10895
MIAQIAALGAVLATNVACASLVEREEAAQFVRMDFDKKRGPSFSEAWSGRAAVPRLAKRNDWVDIEIDNQQMFYSVNLSIGTPPQEVRVLMDTGSSDLWVVGAGVRCGPNNYPNPNQLNCYEHGSFDTSRSSTWKDNNTQFHIRYGDSTYAHGTWGTDRLDLGQANVDGLTFAVAHASNSSVAVLGIGLPAMETTMSSQQFGYQYDNLPMVLKRNNVIKKTVYSMFINELNAKTGSVLFGAVDHSKYKGTLNTVPLVNANYRRRVNKPVELHVTLAAIGLHTGRDEDKVQTLLGSKVPALLDSGTTLTYFPMQLADRLARAAGARWHSEEVGYIVDCNSRRLHQTAYIYDFGGFQIRSPLSDYSMMTNVRGTCRFGIMPHSSDYVILGDVFLTRAYVVFDLEALEVSMAQANYEGGKEQIEVIAGAVPRAVRAPGYNDPWKAYTPLVFNGEFGNLTATASDSSSGSVGSRSNSALTLTPPSLSVMLFAASLLGAATRII

SEQ ID NO: 83 - DNA, 1442 bp, Thielavia terrestris
  misc_feature 1..1442: note = NRRL 8126
  source 1..1442: mol_type = genomic DNA
tgaagtggcacaccttgttggctattgctgcttctttgtctttgtctcaaccagctgatggtttgtctattggtagaagacaagaaggtttgaaggtcttgggtttggaaactcaaagaagagcaccaagaaatccagttcacagagatagattgagaaagagaggtgaattgccaatcggtttggataatgaagaaaccttgtacttcattaacgccactgttggtactccagctactgctttgagattgcatttggatactggttcttcagatatgtgggttaatactccaacttccgcttactgttcttctaagtctaaaccatgtgctttcgctggtacttattctgctaatgcttcttctacctctgaatacgttggttcctacttcaacatctcttacgttgatggttctggtgcttctggtgattatgtttctgatactgttactatcggtggtcacaagatcgatagattgcaatttggtgttggttaccaatctaccaacgctcaaggtattatgggtattggttacccattgaacgaagttcaagttggtagagctggtttgagagcttataacaatttgccagctcaattggttgccgatggtgttattcaatctaaggcttattctttgtggttgaacgatttggatgctaacaccggttctattttgtttggtggtgttgatgctgctaagtacactccaccattattgtctttgccagttgaaccacaatctggtgtttactccgaattcttcattaccttgaccggtttacaattgggttctactgctattggttctgatttggctttggctgttttgttggattctggttcatctttgacttacttgccagattctttggtccaatctatctatgctgctgttggtgctgtttatgattctgatgctaatgctgcttatgttccatgtgctttggcagatgatgcttctgctgctccattgaattttactttcactaccgctaccatttccgttgctatgagagaattggttttggatttggttacctcctctggtcaaagaccaactttttctaatggtgctgctgcttgtttgtttggtattggtccagctggttcaggtgcttcagctggtggttcaggttcttctgcaggtacttctgttttgggtgatacttttttgagatccgcctacgttgtttacgatttggataacaactacgtttctttggctccaaccagattcaactcttctgaatctagagttttggaaatcggtactggtactgctgctgttccaggtgctacaaaagttcaaaatccagttagagctaccgaaggtttgagaggttctggtaatggttcttctgctttgtctgcttcagctgctgctgctgcaggtagaggtgatggtttgggttgggtttctgctcaaaatggtgttggt

SEQ ID NO: 84 - protein, 481 aa, Thielavia terrestris
  REGION 1..481: note = NRRL 8126
MKWHTLLAIAASLSLSQPADGLSIGRRQEGLKVLGLETQRRAPRNPVHRDRLRKRGELPIGLDNEETLYFINATVGTPATALRLHLDTGSSDMWVNTPTSAYCSSKSKPCAFAGTYSANASSTSEYVGSYFNISYVDGSGASGDYVSDTVTIGGHKIDRLQFGVGYQSTNAQGIMGIGYPLNEVQVGRAGLRAYNNLPAQLVADGVIQSKAYSLWLNDLDANTGSILFGGVDAAKYTPPLLSLPVEPQSGVYSEFFITLTGLQLGSTAIGSDLALAVLLDSGSSLTYLPDSLVQSIYAAVGAVYDSDANAAYVPCALADDASAAPLNFTFTTATISVAMRELVLDLVTSSGQRPTFSNGAAACLFGIGPAGSGASAGGSGSSAGTSVLGDTFLRSAYVVYDLDNNYVSLAPTRFNSSESRVLEIGTGTAAVPGATKVQNPVRATEGLRGSGNGSSALSASAAAAAGRGDGLGWVSAQNGVG

SEQ ID NO: 85 - DNA, 1482 bp, Neurospora crassa
  source 1..1482: mol_type = genomic DNA
atgaagagaaccactatttgggaatggattttgaccgcttctttgttgtctactactgaagctttcgccatcagacaaaaacaagatgctgatactccaaagatggtttccttgcaaactgaaagattgtctgttccaaaaccagctgctagagataagttgcaaagaagaggtatgaacgatgttgccttggataatgttattggtggttactacgtcaacgttaccattggtactccaggtagaaatttgtccttgcatttggatactggttcctctgatacttgggttaattccccatcttctatcttgtgtcaagacgaagataagccatgtgaatactctggtacttacttggctaacgattcttctacctacgaatatatctccaaccacttcgatatcaagtacgttgatggttctggtgctagaggtgattatgcttcagatactttcactatcggtaacaccaagttgaacagattgcaattcggtatcggttactcttctactaatgctcaaggtttgttgggtattggttacaccttgtctgaagttcaaacaagagctggtttgccagcttataacaatttgccagcacaaatggttgctgacggtttgattaactctaacgcttactctatctggttgaacgatttggatgctttgaccggtactattttgtttggtggtgttgatgctgctaagtacgaaggtgatttgttgactttgccagttcaaactccagaaaagggtacttacaagaacttgatggttactatgaccggtttgtctttgtcccaatctcaatcttcttcatccgataagggtaatggtgatgataccactcaaatctccaaggataatttggctttggccgttttgttagataccggttctactttgtcttacttgccatctgaattggtcaagccattatacgatgccattggtattgaatatatcaccgatccagatggtaaagtcgatggttatgctccatgtcatttgatgtcatcctctcaatctgtcatgttctcattctcttccccattgcaaattgccgttccaatgaatgaattgatcgtcaacagaaccttccacggtaaattgccaagaatgccagatggtgttactgatgcttgtattttcggtatccaagaaagaaatggtactggtgcaaatactttgggtgatacctttttgagatccgcctacgttgtttttgatttggacaacaacgaaatctccatggctcaaactagattcaatgctactgctaccgacttgaaagaaatcaagaaaggtaaaggtggtgttccaggtgctaaagctgttgaaaatccagttgaagctacttctggtttgtctggtaatgaaggtggtatctatgttaatggtgctgcttgtgaattgaacgttggtatgggtatggcttggggtttgttagttggtgctactatggttgttttgggtttgtga

SEQ ID NO: 86 - protein, 493 aa, Neurospora crassa
MKRTTIWEWILTASLLSTTEAFAIRQKQDADTPKMVSLQTERLSVPKPAARDKLQRRGMNDVALDNVIGGYYVNVTIGTPGRNLSLHLDTGSSDTWVNSPSSILCQDEDKPCEYSGTYLANDSSTYEYISNHFDIKYVDGSGARGDYASDTFTIGNTKLNRLQFGIGYSSTNAQGLLGIGYTLSEVQTRAGLPAYNNLPAQMVADGLINSNAYSIWLNDLDALTGTILFGGVDAAKYEGDLLTLPVQTPEKGTYKNLMVTMTGLSLSQSQSSSSDKGNGDDTTQISKDNLALAVLLDTGSTLSYLPSELVKPLYDAIGIEYITDPDGKVDGYAPCHLMSSSQSVMFSFSSPLQIAVPMNELIVNRTFHGKLPRMPDGVTDACIFGIQERNGTGANTLGDTFLRSAYVVFDLDNNEISMAQTRFNATATDLKEIKKGKGGVPGAKAVENPVEATSGLSGNEGGIYVNGAACELNVGMGMAWGLLVGATMVVLGL

SEQ ID NO: 87 - DNA, 1371 bp, Aspergillus niger
  source 1..1371: mol_type = genomic DNA
atgaagtctaccaccttgttgtctttggcttgggctgctcaatctgcttattctttgtctattcacgaaagagatgaaccagctaccttgcaattcaacttcgaaagaagacaaatcgccgacagatctagaagaaaaagatctactgcttccgccgatttggttaacttggctactaatttgggttacaccatgaacttgactttgggtactccaggtcaagaagtttctgttactttggatactggttcctctgatttgtgggttaatggtgctaattcttctgtttgtccatgtaccgattacggttcttataactcttctgcttcttctacctacaccttcgttaatgacgaattctacatccaatacgtcgatggttctgaagctactggtgattacgttaacgataccttgaagttctctaacgtcactttgaccaatttccaattcgctgttgcttacgatggtgattctgaagaaggtgttttgggtattggttacgcttctaatgaagcttctcaagctacagttggtggtggtgaatataccaattttccagaagctttggttgatcaaggtgctattaactggccagcttactctttgtggttggatgatttggatgaaggtaagggtactattttgttcggtggtgttaataccgctaagtactatggttcattgcaaaccttgccaatcgtcagtattgaagatatgtacgttgaattcgccgttaacttgactgctgttcatttggaaaagaacggtaactctgtttccgttaacaactctgctactcaatttcctattccagccgttttggattctggtactgctttgacttatattccaacttctgctgctgcttcaatctatgaagctgttggtgctcaatacttgtctgaatatggttacggtgttattgaatgcgacgttaaggatgaagatttcaccttcttgttcgacttcggttctttcaacatgtccgttgatatctccgaaatgattttggaagcttcttctgatatgaccgatatgaacgtctgtacttttggtttggccgttattgaaaacgaagctttgttgggtgataccttcttgagatctgcttacgttgtttacgatttgggtaacaacgaaatctctttggctaaggctaatttcaacccaggtgaagatcatgttttggaaataggtactggttctgatgctgttccaaaagctacaggtgctactgcaactggtgctgcagctacttctactgcatcttcagataagtctgacaaagaaagttctgctaccgttccaagatctcaaatcgtttctttggttgccggtgttttagttggtgttttcttggttttgtga

SEQ ID NO: 88 - protein, 456 aa, Aspergillus niger
MKSTTLLSLAWAAQSAYSLSIHERDEPATLQFNFERRQIADRSRRKRSTASADLVNLATNLGYTMNLTLGTPGQEVSVTLDTGSSDLWVNGANSSVCPCTDYGSYNSSASSTYTFVNDEFYIQYVDGSEATGDYVNDTLKFSNVTLTNFQFAVAYDGDSEEGVLGIGYASNEASQATVGGGEYTNFPEALVDQGAINWPAYSLWLDDLDEGKGTILFGGVNTAKYYGSLQTLPIVSIEDMYVEFAVNLTAVHLEKNGNSVSVNNSATQFPIPAVLDSGTALTYIPTSAAASIYEAVGAQYLSEYGYGVIECDVKDEDFTFLFDFGSFNMSVDISEMILEASSDMTDMNVCTFGLAVIENEALLGDTFLRSAYVVYDLGNNEISLAKANFNPGEDHVLEIGTGSDAVPKATGATATGAAATSTASSDKSDKESSATVPRSQIVSLVAGVLVGVFLVL

SEQ ID NO: 89 - DNA, 1638 bp, Bacillus amyloliquefaciens
  source 1..1638: mol_type = genomic DNA
atgcaaaacgccagattattggtctactacaacaacatctccttccactactttcaaaagggtggttttatcgttggtttgggtaagaaattgtctgttgctgttgctgcttctttcatgtctttgactatttctttgccaggtgttcaagctgctgaaaacccacaattgaaagaaaacttgaccaacttcgttccaaagcactctttggttcaatctgaattgccatccgtttctgataaggccattaagcaatacttgaagcaaaacggtaaggttttcaagggtaacccatctgaaagattgaagttgattgatcaaaccaccgatgacttgggttacaagcactttagatatgttccagttgttaacggtgttccagtcaaggattcccaagttattatccacgttgacaagtccaacaacgtttacgctattaacggtgaattgaacaacgatgtttctgctaagactgccaactctaaaaagttgtctgctaatcaagctttggatcatgcttacaaggccattggtaaatcacctgaagctgtttctaatggtactgttgctaacaagaacaaggctgaattgaaagctgctgctactaaggatggtaaatacagattggcttacgatgtcaccatcagatatattgaaccagaaccagctaactgggaagttactgttgatgctgaaactggtaagatcttgaaaaagcaaaacaaggttgaacatgctgctacaactggtactggtactactttgaaaggtaagaccgtttccttgaacatctcttccgaatctggtaagtacgttttgagagatttgtctaagccaaccggtactcaaattatcacttacgacttgcaaaacagagaatacaacttgccaggtactttggtttcttctaccaccaatcaattcactacctcttcacaaagagctgctgttgacgctcattacaatttgggtaaagtttacgactacttctaccaaaagttcaacagaaactcctacgataacaagggtggtaagattgtctcttctgttcattacggttccagatacaacaatgctgcttggattggtgatcaaatgatctatggtgacggtgatggttcttttttctctccattgtctggttctatggatgttactgctcacgaaatgactcatggtgttactcaagaaactgctaacttgaactacgaaaatcaaccaggtgccttgaacgaatctttctctgatgtttttggttacttcaacgacaccgaagattgggatattggtgaagatattaccgtttctcaaccagccttgagatctttgtcaaatccaactaagtatggtcaaccagataacttcaagaactacaagaacttgccaaacactgatgctggtgattatggtggtgttcataccaattctggtattccaaacaaagctgcctacaacaccattaccaaaatcggtgttaacaaggccgaacaaatctactatagagctttgactgtttacttgaccccatcttctacttttaaggatgctaaggctgccttgattcaatctgctagagacttgtatggttctcaagatgctgcttcagttgaagctgcatggaatgctgttggtttgtga

SEQ ID NO: 90 - protein, 545 aa, Bacillus amyloliquefaciens
MQNARLLVYYNNISFHYFQKGGFIVGLGKKLSVAVAASFMSLTISLPGVQAAENPQLKENLTNFVPKHSLVQSELPSVSDKAIKQYLKQNGKVFKGNPSERLKLIDQTTDDLGYKHFRYVPVVNGVPVKDSQVIIHVDKSNNVYAINGELNNDVSAKTANSKKLSANQALDHAYKAIGKSPEAVSNGTVANKNKAELKAAATKDGKYRLAYDVTIRYIEPEPANWEVTVDAETGKILKKQNKVEHAATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 91 - protein, 515 aa, Saccharomycopsis fibuligera
  REGION 1..515: note = glu0111 (GenBank Accession Number: CAC83969.1)
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 92 - protein, 441 aa, synthetic construct
  REGION 1..441: note = Consensus sequence of Figure 4
  VARIANT 1: note = Xaa can be present or absent, when present any naturally occurring amino acid, preferably M
  VARIANT 2: note = Xaa can be present or absent, when present any naturally occurring amino acid, preferably V
  VARIANT 21: note = Xaa can be any naturally occurring amino acid, preferably V, S, T or A
  VARIANT 23: note = Xaa can be any naturally occurring amino acid, preferably V or A
  VARIANT 27: note = Xaa can be any naturally occurring amino acid, preferably P, S, or R
  VARIANT 28: note = Xaa can be any naturally occurring amino acid, preferably K or G
  VARIANT 42: note = Xaa can be any naturally occurring amino acid, preferably K or N
  VARIANT 45: note = Xaa can be present or absent, when present any naturally occurring amino acid, preferably V or N
  VARIANT 46: note = Xaa can be present or absent, when present any naturally occurring amino acid, preferably N
  VARIANT 47: note = Xaa can be present or absent, when present any naturally occurring amino acid, preferably L
  VARIANT 48: note = Xaa can be present or absent, when present any naturally occurring amino acid, preferably P
  VARIANT 49: note = Xaa can be present or absent, when present any naturally occurring amino acid, preferably A
  VARIANT 50: note = Xaa can be present or absent, when present any naturally occurring amino acid, preferably V
  VARIANT 51: note = Xaa can be present or absent, when present any naturally occurring amino acid, preferably Y
  VARIANT 52: note = Xaa can be present or absent, when present any naturally occurring amino acid, preferably A
  VARIANT 53: note = Xaa can be present or absent, when present any naturally occurring amino acid, preferably N
  VARIANT 54: note = Xaa can be present or absent, when present any naturally occurring amino acid, preferably A or V
  VARIANT 55: note = Xaa can be present or absent, when present any naturally occurring amino acid, preferably L or T
  VARIANT 56: note = Xaa can be present or absent, when present any naturally occurring amino acid, preferably T or V
  VARIANT 57: note = Xaa can be any naturally occurring amino acid, preferably K, G or A
  VARIANT 58: note = Xaa can any naturally occurring amino acid, preferably Y, Q or S
  VARIANT 59: note = Xaa can be any naturally occurring amino acid, preferably G, E, D or S
  VARIANT 65: note = Xaa can be present or absent, when present any naturally occurring amino acid, preferably S or N
  VARIANT 66: note = Xaa can be present or absent, when present any naturally occurring amino acid, preferably V or L
  VARIANT 67: note = Xaa can be present or absent, when present any naturally occurring amino acid, preferably K or R
  VARIANT 68: note = Xaa can be present or absent, when present any naturally occurring amino acid, preferably A
  VARIANT 69: note = Xaa can be present or absent, when present any naturally occurring amino acid, preferably A
  VARIANT 70: note = Xaa can be present or absent, when present any naturally occurring amino acid, preferably A or S
  VARIANT 71: note = Xaa can be present or absent, when present any naturally occurring amino acid, preferably S or D
  VARIANT 73: note = Xaa can be any naturally occurring amino acid, preferably G or A
  VARIANT 74: note = Xaa can be any naturally occurring amino acid, preferably S, L, I or V
  VARIANT 83: note = Xaa can be present or absent, when present any naturally occurring amino acid, preferably D
  VARIANT 84: note = Xaa can be any naturally occurring amino acid, preferably S or V
  VARIANT 87: note = Xaa can be any naturally occurring amino acid, preferably L or A
  VARIANT 88: note = Xaa can be any naturally occurring amino acid, preferably T or A
  VARIANT 92: note = Xaa can be any naturally occurring amino acid, preferably V or I
  VARIANT 99: note = Xaa can be any naturally occurring amino acid, preferably L or F
  VARIANT 116: note = Xaa can be present or absent, when present any naturally occurring amino acid, preferably S
  VARIANT 117: note = Xaa can be present or absent, when present any naturally occurring amino acid, preferably V
  VARIANT 118: note = Xaa can be present or absent, when present any naturally occurring amino acid, preferably T
  VARIANT 119: note = Xaa can be present or absent, when present any naturally occurring amino acid, preferably C
  VARIANT 121: note = Xaa can be present or absent, when present any naturally occurring amino acid, preferably K or N
  VARIANT 120: note = Xaa can be present or absent, when present any naturally occurring amino acid, preferably D or E
  VARIANT 122: note = Xaa can be present or absent, when present any naturally occurring amino acid, preferably P
  VARIANT 123: note = Xaa can be present or absent, when present any naturally occurring amino acid, preferably R or P
  VARIANT 124: note = Xaa can be present or absent, when present any naturally occurring amino acid, preferably P
  VARIANT 129: note = Xaa can be any naturally occurring amino acid, preferably S or D
  VARIANT 146: note = Xaa can be any naturally occurring amino acid, preferably K or Q
  VARIANT 147: note = Xaa can be any naturally occurring amino acid, preferably L, N, R or K
  VARIANT 150: note = Xaa can be any naturally occurring amino acid, Y, T, N or S
  VARIANT 162: note = Xaa can be any naturally occurring amino acid, preferably A or S
  VARIANT 163: note = Xaa can be any naturally occurring amino acid, preferably S, Q, H or R
  VARIANT 165: note = Xaa can be any naturally occurring amino acid, preferably D or T
  VARIANT 191: note = Xaa can be present or absent, when present any naturally occurring amino acid, preferably S
  VARIANT 192: note = Xaa can be present or absent, when present any naturally occurring amino acid, preferably Q
  VARIANT 193: note = Xaa can be present or absent, when present any naturally occurring amino acid, preferably F
  VARIANT 194: note = Xaa can be present or absent, when present any naturally occurring amino acid, preferably V
  VARIANT 195: note = Xaa can be present or absent, when present any naturally occurring amino acid, preferably Q
  VARIANT 196: note = Xaa can be present or absent, when present any naturally occurring amino acid, preferably D
  VARIANT 197: note = Xaa can be present or absent, when present any naturally occurring amino acid, preferably K
  VARIANT 202: note = Xaa can be any naturally occurring amino acid, preferably L or I
  VARIANT 215: note = Xaa can be present or absent, when present any naturally occurring amino acid, preferably Q
  VARIANT 216: note = Xaa can be present or absent, when present any naturally occurring amino acid, preferably S
  VARIANT 217: note = Xaa can be present or absent, when present any naturally occurring amino acid, preferably S
  VARIANT 218: note = Xaa can be present or absent, when present any naturally occurring amino acid, preferably N
  VARIANT 219: note = Xaa can be present or absent, when present any naturally occurring amino acid, preferably S
  VARIANT 220: note = Xaa can be present or absent, when present any naturally occurring amino acid, preferably F
  VARIANT 231: note = Xaa can be any naturally occurring amino acid, preferably V, N, K or D
  VARIANT 236: note = Xaa can be any naturally occurring amino acid, preferably D or A
  VARIANT 262: note = Xaa can be any naturally occurring amino acid, preferably S or A
  VARIANT 272: note = Xaa can be any naturally occurring amino acid, preferably D or P
  VARIANT 273: note = Xaa can be present or absent, when present any naturally occurring amino acid, preferably L
  VARIANT 280: note = Xaa can be present or absent, when present any naturally occurring amino acid, preferably G
  VARIANT 281: note = Xaa can be present or absent, when present any naturally occurring amino acid, preferably D
  VARIANT 282: note = Xaa can be present or absent, when present any naturally occurring amino acid, preferably S
  VARIANT 283: note = Xaa can be present or absent, when present any naturally occurring amino acid, preferably T
  VARIANT 284: note = Xaa can be present or absent, when present any naturally occurring amino acid, preferably S
  VARIANT 285: note = Xaa can be present or absent, when present any naturally occurring amino acid, preferably G
  VARIANT 286: note = Xaa can be present or absent, when present any naturally occurring amino acid, preferably A
  VARIANT 287: note = Xaa can be present or absent, when present any naturally occurring amino acid, preferably V
  VARIANT 288: note = Xaa can be present or absent, when present any naturally occurring amino acid, preferably F or A
  VARIANT 295: note = Xaa can be any naturally occurring amino acid, preferably G or S
  VARIANT 306: note = Xaa can be present or absent, when present any naturally occurring amino acid, preferably N or T
  VARIANT 318: note = Xaa can be any naturally occurring amino acid, preferably L or I
  VARIANT 324: note = Xaa can be any naturally occurring amino acid, preferably S or D
  VARIANT 331: note = Xaa can be any naturally occurring amino acid, preferably R or D
  VARIANT 334: note = Xaa can be any naturally occurring amino acid, preferably S, Q, H or G
  VARIANT 342: note = Xaa can be any naturally occurring amino acid, preferably Y, Q, N or G
  VARIANT 344: note = Xaa can be present or absent, when present any naturally occurring amino acid, preferably G, H or N
  VARIANT 345: note = Xaa can be present or absent, when present any naturally occurring amino acid, preferably T or S
  VARIANT 346: note = Xaa can be present or absent, when present any naturally occurring amino acid, preferably F or L
  VARIANT 356: note = Xaa can be present or absent, when present any naturally occurring amino acid, preferably T
  VARIANT 357: note = Xaa can be any naturally occurring amino acid, preferably P or V
  VARIANT 362: note = Xaa can be any naturally occurring amino acid, preferably V, D, A or S
  VARIANT 365: note = Xaa can be present or absent, when present any naturally occurring amino acid, preferably G
  VARIANT 366: note = Xaa can be any naturally occurring amino acid, preferably Y, V, A or K
  VARIANT 377: note = Xaa can be present or absent, when present any naturally occurring amino acid, preferably A
  VARIANT 378: note = Xaa can be present or absent, when present any naturally occurring amino acid, preferably P or S
  VARIANT 380: note = Xaa can be any naturally occurring amino acid, preferably Y, S, F or L
  VARIANT 382: note = Xaa can be any naturally occurring amino acid, preferably P, A, T or Q
  VARIANT 386: note = Xaa can be any naturally occurring amino acid, preferably G or P
  VARIANT 388: note = Xaa can be any naturally occurring amino acid, preferably S or P
  VARIANT 389: note = Xaa can be any naturally occurring amino acid, preferably T, K, Q or E
  VARIANT 393: note = Xaa can be any naturally occurring amino acid, preferably G or L
  VARIANT 396: note = Xaa can be any naturally occurring amino acid, preferably S or I
  VARIANT 397: note = Xaa can be present or absent, when present any naturally occurring amino acid, preferably N
  VARIANT 399: note = Xaa can be any naturally occurring amino acid, preferably G or D
  VARIANT 402: note = Xaa can be present or absent, when present any naturally occurring amino acid, preferably F or Y
  VARIANT 403: note = Xaa can be present or absent, when present any naturally occurring amino acid, preferably S or Y
  VARIANT 422: note = Xaa can be any naturally occurring amino acid, preferably P, D, N or S
  VARIANT 423: note = Xaa can be any naturally occurring amino acid, preferably R, K,E, or Q
  VARIANT 425: note = Xaa can be any naturally occurring amino acid, preferably G or S
  VARIANT 432: note = Xaa can be present or absent, when present any naturally occurring amino acid, preferably T
  VARIANT 433: note = Xaa can be present or absent, when present any naturally occurring amino acid, preferably S
  VARIANT 434: note = Xaa can be present or absent, when present any naturally occurring amino acid, preferably A
  VARIANT 435: note = Xaa can be present or absent, when present any naturally occurring amino acid, preferably S
  VARIANT 436: note = Xaa can be present or absent, when present any naturally occurring amino acid, preferably N
  VARIANT 437: note = Xaa can be present or absent, when present any naturally occurring amino acid, preferably I
  VARIANT 438: note = Xaa can be present or absent, when present any naturally occurring amino acid, preferably A
  VARIANT 439: note = Xaa can be present or absent, when present any naturally occurring amino acid, preferably A
  VARIANT 440: note = Xaa can be present or absent, when present any naturally occurring amino acid, preferably L
  VARIANT 441: note = Xaa can be present or absent, when present any naturally occurring amino acid, preferably T
XXMFLKNIFIALALALLVDAXPXKRSXXFVTLDFDVIKTPVXATXXXXXXXXXXXXGQXGKVKRXXXXXXXQXXPVTLNNEYXXSYXXDITXGSNGQKXNVDVDTGSSDLWVPDAXXXXXXXXXGQSAXFCKGKGIYTPKSSTTSXXLGXPFYIGYGDGSSXXGXLYKDTVGFGGASITKQVFADATKTSXXXXXXXVNQGXLGIGYKTNEAAGXXXXXXDYDNVPVTLKXQGVIXKNAYSLYLNSPNAATGQIIFGGVDKXKYSGSLIAVXXVTSDREXXXXXXXXXLRITLNXIKAGGKNINGXNIDVLLDSGTTXTYLQQXVAQDIIXAFXAELKSDGXGXXXYVTDCQTSGXXDFNFXNNXXKISVPASEFTXXLXYXNGQXYXXCQLXLGXXSXANXXILGDNFLRSAYVVYDLDDXXIXLAQVKYXXXXXXXXXX